(19) United States
(12) Patent Application Publication
(10) Pub. No.: US 2008/0207685 A1
(43) Pub. Date: Aug. 28, 2008

(54) Heterocyclic Compounds As Modulators Of Peroxisome Proliferator Activated Receptors, Useful For The Treatment And/Or Prevention Of Disorders Modulated By A Ppar

(75) Inventors: James Robert Henry, Indianapolis, IN (US); Yihong Li, Carmel, IN (US); Jeffrey Michael Scheryantz, Fishers, IN (US); Alan M. Warshawsky, Carmel, IN (US)

Correspondence Address: ELI LILLY & COMPANY, PATENT DIVISION, P.O. BOX 6288, INDIANAPOLIS, IN, 46206-6288, US

(73) Assignee: Eli Lilly And Company, Indianapolis, IN (US)

(21) Appl. No.: 10/577,828

(22) PCT Filed: Nov. 16, 2004
(86) PCT No.: PCT/US2004/035530

§ 371 Date: May 1, 2006

Related U.S. Application Data
(60) Provisional application No. 60/523,906, filed on Nov. 20, 2003.

(51) Int. Cl. A61K 31/4709; C07D 217/24; A61P 3/10; A61P 9/10; A61P 3/04

(52) U.S. Cl. 514/309; 546/141

(57) ABSTRACT

The present invention is directed to a compound of formula (I), or a pharmaceutically acceptable salt, solvate, hydrate or stereoisomer thereof, which is useful in treating or preventing disorders mediated by a peroxisome proliferator activated receptor (PPAR) such as syndrome X, type II diabetes, hyperglycemia, hyperlipidemia, obesity, coagaulopathy, hypertension, arteriosclerosis, and other disorders related to syndrome X and cardiovascular diseases.

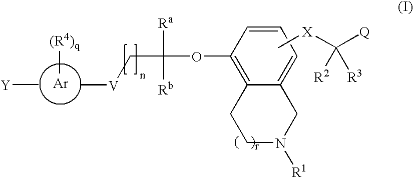

### FIELD OF THE INVENTION

The present invention relates to a compound of peroxisome proliferator activated receptor (PPAR) agonists, which are useful for the treatment and/or prevention of disorders modulated by a PPAR.

### BACKGROUND OF THE INVENTION

The peroxisome proliferator activated receptors (PPARs) are members of the nuclear receptor gene family that are activated by fatty acids and fatty acid metabolites. The PPARs belong to the subset of nuclear receptors that function as heterodimers with the 9-cis retinoic acid receptor (RXR). Three subtypes, designated PPARα, PPARγ and PPARδ, are found in species ranging from Xenopus to humans.

PPARα is the main subtype in the liver and has facilitated analysis of the mechanism by which peroxisome proliferators exert their pleiotropic effects. PPARα is activated by a number of medium and long-chain fatty acids, and it is involved in stimulating β-oxidation of fatty acids. PPARα is also involved with the activity of fibrates and fatty acids in rodents and humans. Fibric acid derivatives such as clofibrate, fenofibrate, bezafibrate, ciprofibrate, beclofibrate and etofibrate, as well as gemfibrozil, produce a substantial reduction in plasma triglycerides along with moderate reduction in low-density lipoprotein (LDL) cholesterol, and they are used particularly for the treatment of hypertriglyceridemia.

PPARγ is the main subtype in adipose tissue and involved in activating the program of adipocyte differentiation. PPARγ is not involved in stimulating peroxisome proliferation in the liver. There are two isomers of PPARγ: PPARγ1 and PPARγ2, which differ only in that PPARγ2 contains an additional 28 amino acids present at the amino terminus. The DNA sequences for the PPARγ receptors are described in Elbrecht, et al., BBRC 224; 431-437 (1996). Although peroxisome proliferators, including the fibrates and fatty acids, activate the transcriptional activity of PPAR's, only prostaglandin J₂ derivatives have been identified as natural ligands for PPARγ, which also binds the anti-diabetic agents thiazolidinediones with high affinity. The physiological functions of PPARα and PPARγ in lipid and carbohydrate metabolism were uncovered once it was recognized that they were the receptors for the fibrate and glitazone drugs, respectively.

PPARα and PPARγ receptors have been implicated in diabetes mellitus, cardiovascular disease, obesity, and gastrointestinal disease, such as inflammatory bowel disease and other inflammation related illnesses. Such inflammation related illnesses include, but are not limited to Alzheimer's disease, Crohn's disease, rheumatoid arthritis, psoriasis, and ischemia reprofusion injury.

By contrast, PPARδ (also referred to as PPARδ and NUC1) is not reported to be receptor for any known class of drug molecules, and its role in mammalian physiology has remained undefined. The human nuclear receptor gene PPARδ (hPPARδ) has been cloned from a human osteosarcoma cell cDNA library and is fully described in A. Schmidt et al., Molecular Endocrinology, 6:1634-1641 (1992).

Diabetes is a disease in which a mammal's ability to regulate glucose levels in the blood is impaired because the mammal has a reduced ability to convert glucose to glycogen for storage in muscle and liver cells. In Type I diabetes, this reduced ability to store glucose is caused by reduced insulin production. “Type II Diabetes” or “non-insulin dependent diabetes mellitus” (NIDDM) is the form of diabetes, which is due to a profound resistance to insulin stimulating or regulatory effect on glucose and lipid metabolism in the main insulin-sensitive tissues, muscle, liver and adipose tissue. This resistance to insulin responsiveness results in insufficient insulin activation of glucose uptake, oxidation and storage in muscle and inadequate insulin repression of lipolysis in adipose tissue and of glucose production and secretion in liver. When these cells become desensitized to insulin, the body tries to compensate by producing abnormally high levels of insulin and hyperinsulemia results. Hyperinsulemia is associated with hypertension and elevated body weight. Since insulin is involved in promoting the cellular uptake of glucose, amino acids and triglycerides from the blood by insulin sensitive cells, insulin insensitivity can result in elevated levels of triglycerides and LDL (known as the “bad” cholesterol) which are risk factors in cardiovascular diseases. The constellation of symptoms, which includes hyperinsulemia, combined with hypertension, elevated body weight, elevated triglycerides and elevated LDL is known as Syndrome X.

Hyperlipidemia is a condition, which is characterized by an abnormal increase in serum lipids, such as cholesterol, triglycerides and phospholipids. These lipids do not circulate freely in solution in plasma, but are bound to proteins and transported as macromolecular complexes called lipoproteins. One form of hyperlipidemia is hypercholesterolemia, characterized by the existence of elevated LDL cholesterol levels. The initial treatment for hypercholesterolemia is often a diet low in fat and cholesterol coupled with appropriate physical exercise. Drug intervention is initiated if LDL-lowering goals are not met by diet and exercise alone. It is desirable to lower elevated levels of LDL cholesterol and increase levels of HDL cholesterol. Generally, it has been found that increased levels of HDL are associated with lower risk for coronary heart disease (CHD). See Gordon, et al., Am. J. Med., 62, 707-714 (1977); Stampfer, et al., N. England J. Med., 325, 373-381 (1991); and Kannel, et al., Ann. Internal Med., 90, 85-91 (1979). An example of an HDL raising agent is nicotinic acid, but the quantities needed to achieve HDL elevation are associated with undesirable effects, such as flushing.

There are several treatments currently available for treating diabetes mellitus but these treatments still remain unsatisfactory and have limitations. While physical exercise and reduction in dietary intake of calories will improve the diabetic condition, compliance with this approach can be poor because of sedentary lifestyles and excess food consumption, in particular high fat-containing food. Therefore, treatment with hypoglycemics, such as sulfonylureas (e.g., chlorpropamide, tolbutamide, tolazamide and acetohexamide) and biguanides (e.g. phenformin and metformin) are often necessary as the disease progresses. Sulfonylureas stimulate the β cells of the pancreas to secrete more insulin as the disease progresses. However, the response of the P cells eventually fails and treatment with insulin injections is necessary. In addition, both sulfonylurea treatment and insulin injection have the life threatening side effect of hypoglycemic coma, and thus patients using these treatments must carefully control dosage.

It has been well established that improved glycemic control in patients with diabetes (Type I and Type II) is accompanied by decreased microvascular complications (DCCT and UKPDS). Due to difficulty in maintaining adequate glycemic control over time in patients with Type II diabetes, the use of insulin sensitizers in the therapy of Type II diabetes is growing. There is also a growing body of evidence that PPARγ agonist, insulin sensitizer, may have benefits in the treatment of Type II diabetes beyond their effects in improving glycemic control.

In the last decade a class of compounds known as thiazolidinediones (TZD) (e.g. U.S. Pat. Nos. 5,089,514; 4,342,771; 4,367,234; 4,340,605; and 5,306,726) have emerged as effective antidiabetic agents that have been shown to increase the sensitivity of insulin sensitive tissues, such as skeletal muscle, liver and adipose, to insulin. Increasing insulin sensitivity rather than the amount of insulin in the blood reduces the likelihood of hypoglycemic coma. Although thiazolidinediones have been shown to increase insulin sensitivity by binding to PPARγ receptors, this treatment also produces unwanted side effects such as weight gain and, for troglitazone, liver toxicity.

Recently, compounds that are not TZDs have also been reported.

Adams et al. (WO 97/28115, WO 97/28135 and U.S. Pat. No. 5,895,051) discloses acetylphenols, which are useful as antiobesity and antidiabetic compounds.

Leibowitz et al. (WO 97/28149) discloses compounds which are PPARδ agonists and useful for treating cardiovascular diseases and related conditions.

Brooks et al. (WO 02/100813) discloses compounds of PPAR modulators that are useful for treating type II diabetes and other PPAR-mediated diseases and conditions.

In view of the above, an objective of the present invention is to provide new pharmaceutical agents, which modulate PPAR receptors, to prevent, treat and/or alleviate these diseases or conditions while reducing and or eliminating one or more of the unwanted side effects associated with the current treatments.

### SUMMARY OF THE INVENTION

The present invention relates to a compound of novel peroxisome proliferator activated receptor agonists having a structural formula I,

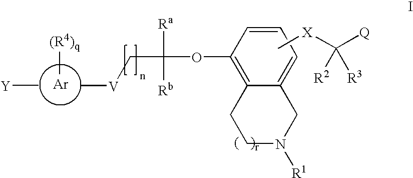

or a pharmaceutically acceptable salt or stereoisomer thereof, wherein:
V is: a bond or O;

### X is: CH₂ or O;

### Q is: C(O)OR⁵ or R^{5A};

n is: 0, 1, 2, 3, or 4
m and q are each independently: 1, 2, 3 or 4;
p is: 1 or 2;
r is: 0 or 1;

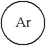

is: aryl, or 5- or 6-membered heteroaryl;

### Y is:

hydrogen,
aryloxy,
cycloalkyl,
heterocyclyl optionally being substituted with heteroaryl, heteroaryl optionally being substituted with aryl,
(C₀-C₄)alkyl-aryl, wherein aryl being optionally substituted with aryl, aryloxy, heteroaryl, heterocyclyl or cycloalkyl, or
aryl-O(CH₂)ᵣ-aryl;
wherein aryl, cycloalkyl, aryloxy, heteroaryl, and heterocyclyl are optionally substituted with one or more substituents independently selected from R⁶;
   R^{a} and R^{b} are each independently: hydrogen or C₁-C₄ alkyl;
   R¹ is: hydrogen,
alkyl,
aryl,
biphenyl,
C(O)ₚ-alkyl,
C(O)ₚ-alkynyl,
C(O)ₚ-alkoxy,
C(O)ₚ(C₀-C₅)alkyl-cycloalkyl,
C(O)ₚ-haloalkyl,
C(O)ₚ-biphenyl,
C(O)ₚ(C₀-C₅)alkyl-aryl,
C(O)ₚ(C₀-C₅)alkyl-heteroaryl,
C(O)ₚ(CH₂)ₘ-aryloxy,
C(O)ₚ(CH₂)ₘ—SR⁷,
C(O)ₚC(R⁷)(aryl)₂,
C(O)N(R⁷)₂,
S(O)ₚ-alkyl,
S(O)ₚ(C₀-C₆)alkyl-aryl or
S(O)ₚ(C₀-C₆)alkyl-heteroaryl;
wherein alkyl, aryl, aryloxy, alkynyl, alkoxy, cycloalkyl, heteroaryl and biphenyl being optionally substituted with one or more substituents independently selected from R^{6a};
   R² and R³ are each independently: hydrogen, C₀-C₆ alkyl or C₁-C₆ alkoxy;
   R⁴ is: hydrogen,
C₁-C₆ alkyl,
C₁-C₆ alkoxy,
halo,
haloalkyl or
haloalkyloxy;
   R⁵ is: hydrogen, C₁-C₆alkyl or aminoalkyl;
   R^{5A} is: carboxamide, sulfonamide, acylsulfonamide, tetrazole,

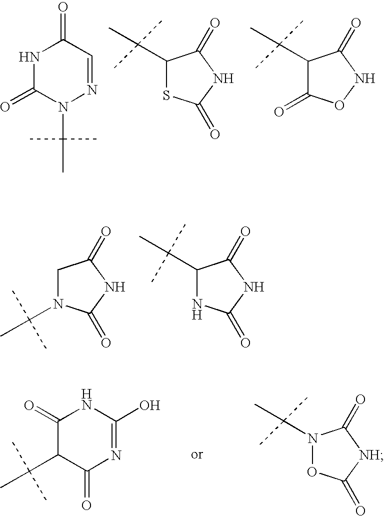

R⁶ and R^{6a} are each independently:
hydrogen,
halo,
nitro,
acyl,
cyano,
hydroxyl,
haloalkyl,
haloalkyloxy,
phenyl,
phenoxy,
benzyloxy,
thiophene,
pyridyl,
C₁-C₆ alkyl,
C₁-C₆ alkoxy,
S(O)₂R⁷,
S(O)₂N(R⁷)₂,
SR⁷ or
N(R⁷)₂; and
   R⁷ is: hydrogen, C₁-C₆ alkyl or (C₀-C₆-alkyl)-aryl.

The compounds of the present invention are useful in the treatment or prevention of diseases or condition relates to hyperglycemia, dyslipidemia, Type II diabetes, Type I diabetes, hypertriglyceridemia, syndrome X, insulin resistance, heart failure, diabetic dyslipidemia, hyperlipidemia, hypercholesteremia, hypertension, obesity, anorexia bulimia, anorexia nervosa, cardiovascular disease and other diseases where insulin resistance is a component.

In one embodiment, the present invention also relates to a pharmaceutical composition comprising a compound of the present invention, or a pharmaceutically acceptable salt, solvate or hydrate thereof and a pharmaceutically acceptable carrier. Within the scope of this invention also include a pharmaceutical composition containing additional therapeutic agent as well as a compound of the present invention, or a pharmaceutically acceptable salt, solvate or hydrate thereof and a pharmaceutically acceptable carrier.

In another embodiment, the present invention relates to a method of modulating a PPAR by contacting the receptor with a compound of the present invention, or a pharmaceutically acceptable salt, solvate or hydrate thereof.

### DETAILED DESCRIPTION OF THE INVENTION

The compounds of the present invention are directed to peroxisome proliferator activated receptor (PPAR) agonists, more specifically compounds of isoindole and isoquinoline derivatives as PPAR modulators and PPAR-γ agonists. The compounds of the present invention are useful for the treatment and/or prevention of disorders modulated by a PPAR, such as Type II diabetes, hyperglycemia, dyslipidemia, Type I diabetes, hypertriglyceridemia, syndrome X, insulin resistance, heart failure, diabetic dyslipidemia, hyperlipidemia, hypercholesteremia, hypertension, obesity, anorexia bulimia, anorexia nervosa, cardiovascular disease and other related diseases.

An embodiment of the present invention is a compound of novel peroxisome proliferator activated receptor (PPAR) agonists having a structural formula I,

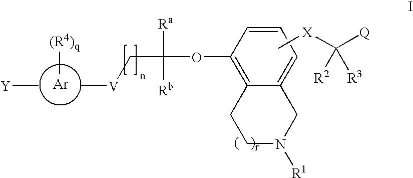

or a pharmaceutically acceptable salt or stereoisomers thereof, wherein:
V is: a bond or O;

### X is: CH₂ or O;

### P Q is: C(O)OR⁵ or R^{5A};

n is: 0, 1, 2, 3 or 4;
m and q are each independently: 1, 2, 3 or 4;
p is: 1 or 2;
r is: 0 or 1;

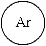

is: aryl, or 5- or 6-membered heteroaryl;
Y is: hydrogen,
aryloxy,
cycloalkyl,
heterocyclyl optionally being substituted with heteroaryl,
heteroaryl optionally being substituted with aryl,
(C₀-C₄)alkyl-aryl, wherein aryl being optionally substituted with aryl, aryloxy, heteroaryl, heterocyclyl or cycloalkyl, or
aryl-O(CH₂)ₘ-aryl;
wherein aryl, cycloalkyl, aryloxy, heteroaryl, and heterocyclyl are optionally substituted with one or more substituents independently selected from R⁶;
   R^{a} and R^{b} are each independently: hydrogen or C₁-C₄ alkyl;
   R¹ is: hydrogen,
alkyl,
aryl,
biphenyl,
C(O)ₚ-alkyl,
C(O)ₚ-alkynyl,
C(O)ₚ-alkoxy,
C(O)ₚ(C₀-C₅)alkyl-cycloalkyl,
C(O)ₚ-haloalkyl,
C(O)ₚ-biphenyl,
C(O)ₚ(C₀-C₅)alkyl-aryl,
C(O)ₚ(C₀-C₅)alkyl-heteroaryl,
C(O)ₚ(CH₂)ₘ-aryloxy,
C(O)ₚ(CH₂)ₘ—SR⁷,
C(O)ₚC(R⁷)(aryl)₂,
C(O)N(R⁷)₂,
S(O)ₚ-alkyl,
S(O)ₚ(C₀-C₆)alkyl-aryl or
S(O)ₚ(C₀-C₆)alkyl-heteroaryl;
wherein alkyl, aryl, aryloxy, alkynyl, alkoxy, cycloalkyl, heteroaryl and biphenyl being optionally substituted with one or more substituents independently selected from R^{6a};
   R² and R³ are each independently: hydrogen, C₁-C₆ alkyl or C₁-C₆ alkoxy;
   R⁴ is: hydrogen,
C₁-C₆ alkyl,
C₁-C₆ alkoxy,
halo,
haloalkyl or
haloalkyloxy;
   R⁵ is: hydrogen, C₁-C₆ alkyl or aminoalkyl;
   R^{5A} is: carboxamide, sulfonamide, acylsulfonamide, tetrazole,

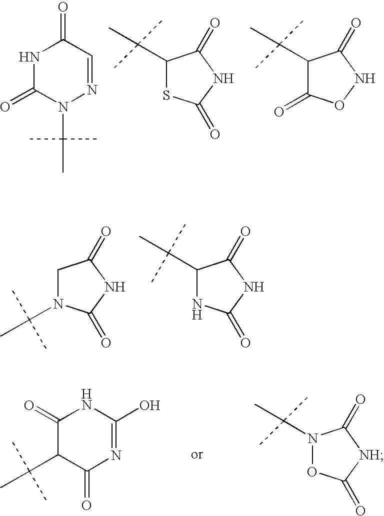

R⁶ and R^{6a} are each independently:
hydrogen,
halo,
nitro,
acyl,
cyano,
hydroxyl,
haloalkyl,
haloalkyloxy,
phenyl,
phenoxy,
benzyloxy,
thiophene,
pyridyl,
C₁-C₆ alkyl,
C₁-C₆ alkoxy,
S(O)₂R⁷,
S(O)₂N(R⁷)₂,
SR⁷ or
N(R⁷)₂; and
   R⁷ is: hydrogen, C₁-C₆ alkyl or (C₀-C₆-alkyl)-aryl.

The compound as recited above, wherein

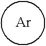

is phenyl, oxazolyl, thiazolyl, pyrazolyl or hydrofuranyl.

A preferred embodiment of the present invention is a compound having a structural formula II,

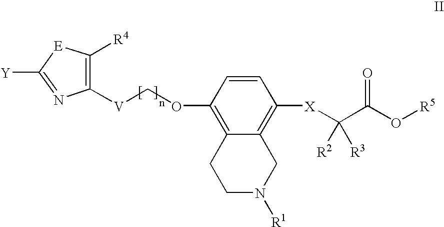

or a pharmaceutically acceptable salt or stereoisomer thereof, wherein:

### E is O or S;

### Y is:

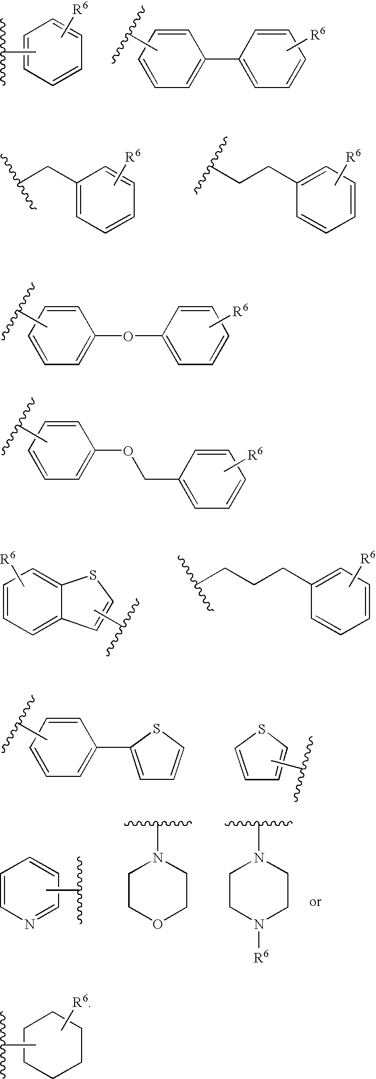

The compound of formula II as recited above, wherein E is O.

Another preferred embodiment of the present invention is a compound having a structural formula III,

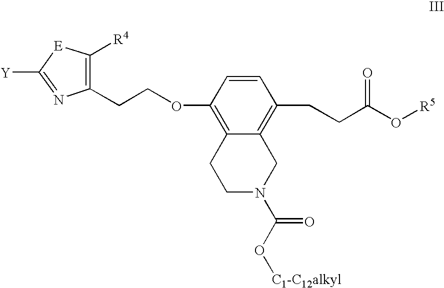

or a pharmaceutically acceptable salt or stereoisomer thereof, wherein:
R⁴ and R⁵ are each independently hydrogen, methyl or ethyl.

Yet another preferred embodiment of the present invention is a compound having a structural formula IV,

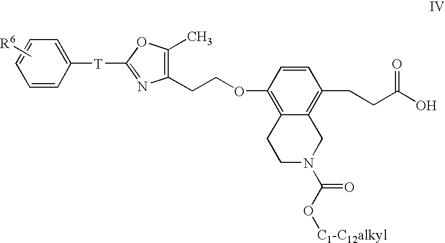

or a pharmaceutically acceptable salt or stereoisomer thereof, wherein:
T is: a bond, CH₂, (CH₂)₂, or (CH₂)₃,
R⁶ is: hydrogen, F, Br or CF₃, OCF₃, thiophene, benzyloxy, phenyl or pyridyl; and
C₁-C₁₂ alkyl is selected from the group consisting of:
methyl, ethyl, propyl, tert-butyl, butyl, isobutyloctane, hexyl, 2-hexylethyl, octyl, and 2,2-dimethylpropyl.

Yet another preferred embodiment of the present invention is a compound having a structural formula V,

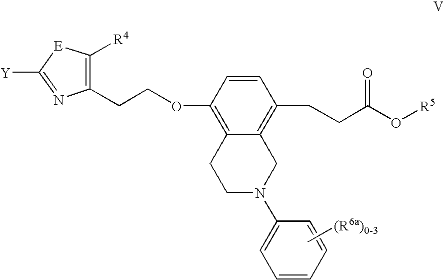

or a pharmaceutically acceptable salt or stereoisomer thereof, wherein:
R⁴ and R⁵ are each independently hydrogen, methyl or ethyl.

Yet another preferred embodiment of the present invention is a compound having a structural formula VI,

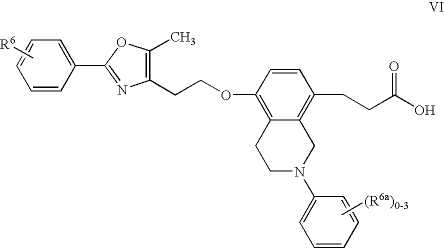

or a pharmaceutically acceptable salt or stereoisomer thereof, wherein:
R⁶ is: hydrogen, F, Br or CF₃, OCF₃, thiophene, benzyloxy, phenyl or pyridyl; and
R^{6a} is each independently selected from the group consisting of:
methyl, ethyl, propyl, isopropyl, tert-butyl, pentyl, 1,1-dimethylpropyl, methoxy, butoxy, acetyl, propionyl, phenyl, methanesulfonyl, F, Cl, Br, CF₃, OCF₃, nitro, cyano, dimethylamino and ethylsunfanyl.

Yet another preferred embodiment of the present invention is a compound having a structural formula VII,

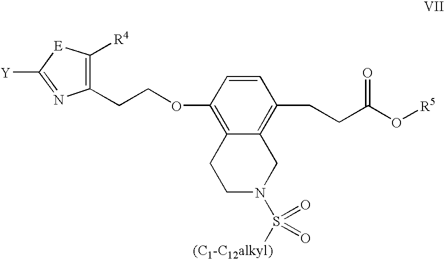

or a pharmaceutically acceptable salt or stereoisomer thereof, wherein:
R⁴ and R⁵ are each independently hydrogen, methyl or ethyl.

Yet another preferred embodiment of the present invention is a compound having a structural formula VIII,

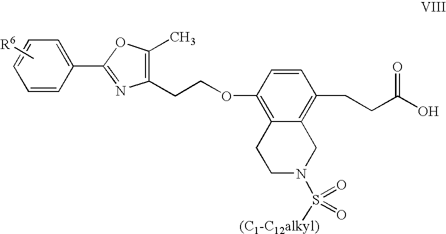

or a pharmaceutically acceptable salt or stereoisomer thereof, wherein:
R⁶ is: hydrogen, F, Br or CF₃, OCF₃, thiophene, benzyloxy, phenyl or pyridyl;
C₁-C₁₂ alkyl is selected from the group consisting of:
methyl, ethyl, propyl, tert-butyl, butyl, isobutyl, octane, and 2,2-dimethylpropyl;

Yet another preferred embodiment of the present invention is a compound having a structural formula IX,

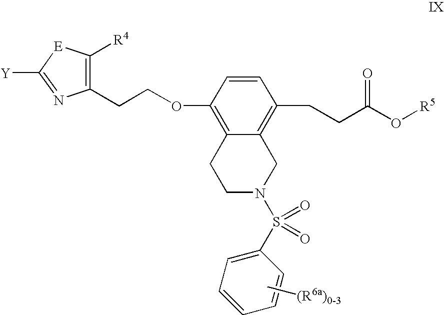

or a pharmaceutically acceptable salt or stereoisomer thereof, wherein:
R⁴ and R⁵ are each independently hydrogen or methyl.

Yet another preferred embodiment of the present invention is a compound having a structural formula X,

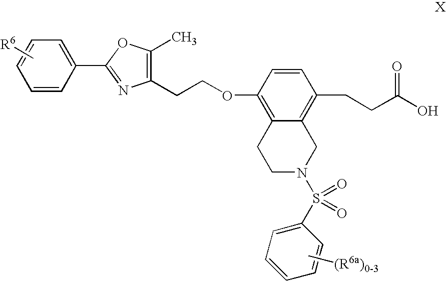

or a pharmaceutically acceptable salt or stereoisomer thereof, wherein:
R⁶ is hydrogen, F, Br or CF₃, OCF₃, thiophene, benzyloxy, phenyl or pyridyl; and
R^{6a} is each independently selected from the group consisting of:
methyl, ethyl, propyl, isopropyl, tert-butyl, pentyl, 1,1-dimethylpropyl, methoxy, butoxy, acetyl, propionyl, phenyl, methanesulfonyl, F, Cl, Br, CF₃, OCF₃, nitro, cyano, dimethylamino and ethylsunfanyl.

Yet another preferred embodiment of the present invention is the compound having a structural formula XI,

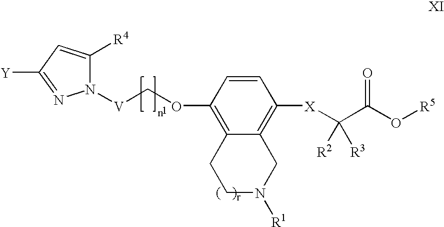

or a pharmaceutically acceptable salt or stereoisomer thereof, wherein:

### Y is:

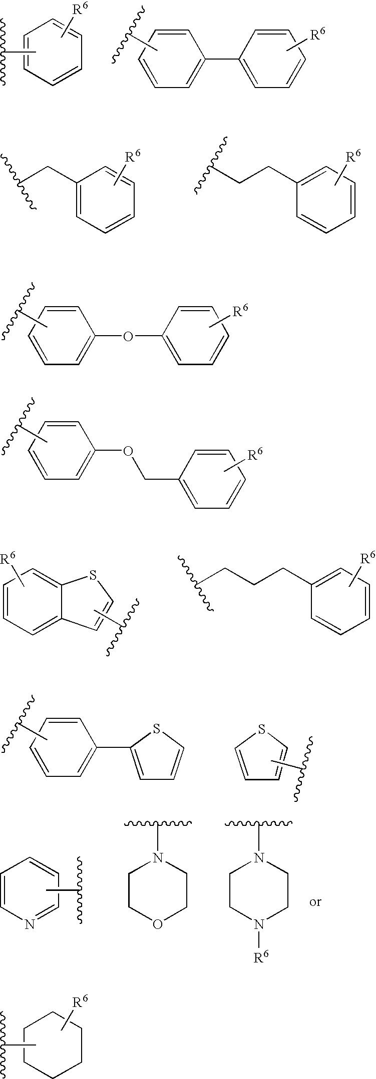

Yet another preferred embodiment of the present invention is the compound having a structural formula XII,

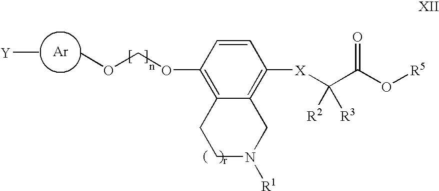

or a pharmaceutically acceptable salt or stereoisomer thereof.

Yet another preferred embodiment of the present invention is the compound having a structural formula XIII,

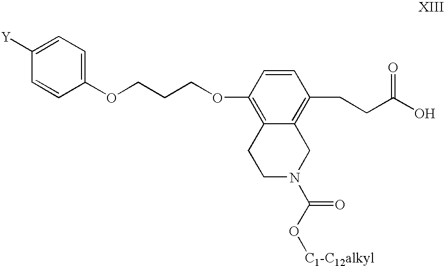

or a pharmaceutically acceptable salt or stereoisomer thereof, wherein:
Y is: phenyl or phenoxy; and
C₁-C₁₂ alkyl is selected from the group consisting of:
methyl, ethyl, propyl, tert-butyl, butyl, isobutyl, octyl and 2,2-dimethylpropyl.

Yet another preferred embodiment of the present invention is the compound having a structural formula XIV,

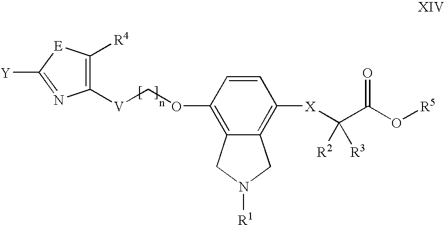

or a pharmaceutically acceptable salt or stereoisomer thereof, wherein:

### E is O or S.

Yet another preferred embodiment of the present invention is the compound having a structural formula XV,

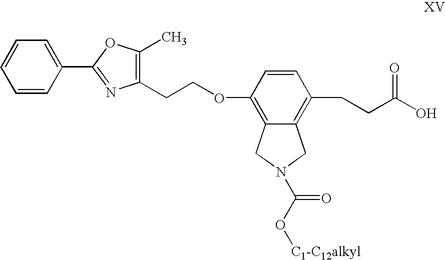

or a pharmaceutically acceptable salt or stereoisomer thereof, wherein:
C₁-C₁₂ alkyl is selected from the group consisting of:
methyl, ethyl, propyl, tert-butyl, butyl, isobutyl, octyl, hexyl, 2-hexylethyl, and 2,2-dimethylpropyl.

Yet another preferred embodiment of the present invention is the compound having a structural formula XVI,

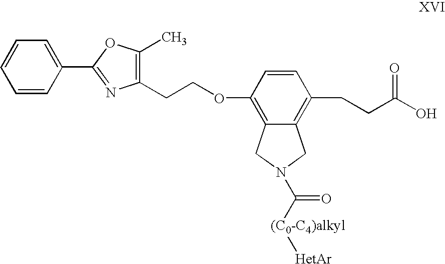

or a pharmaceutically acceptable salt or stereoisomer thereof, wherein:
HetAr is: phenyl, thiophene, pyridine or pyrazine, wherein HetAr being optionally substituted with one to three substituents selected from the group consisting of: halo, methyl, ethyl, methoxy and ethoxy.

Yet another preferred embodiment of the present invention is the compound having a structural formula XVII,

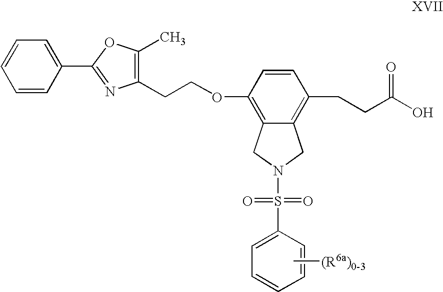

or a pharmaceutically acceptable salt or stereoisomer thereof, wherein:
R⁶ is each independently selected from the group consisting of:
methyl, ethyl, propyl, isopropyl, tert-butyl, pentyl, 1,1-dimethylpropyl, methoxy, butoxy, acetyl, propionyl, phenyl, methanesulfonyl, F, Cl, Br, CF₃, OCF₃, nitro, cyano, dimethylamino and ethylsunfanyl.

Yet another preferred embodiment of the present invention is the compound having a structural formula XVIII,

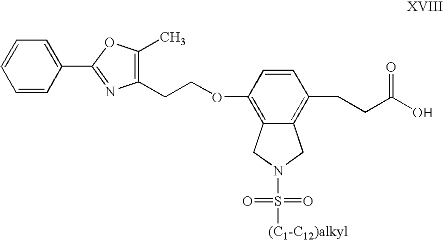

or a pharmaceutically acceptable salt or stereoisomer thereof, wherein:
C₁-C₁₂ allyl is selected from the group consisting of:
methyl, ethyl, propyl, tert-butyl, butyl, isobutyl, octyl, hexyl, 2-hexylethyl, and 2,2-dimethylpropyl.

Also encompassed by the present invention is a pharmaceutical composition comprising a pharmaceutically acceptable carrier and a compound of the present invention or a pharmaceutically acceptable salt, solvate or hydrate thereof.

Also encompassed by the present invention is a pharmaceutical composition comprising: (1) a compound of the present invention or a pharmaceutically acceptable salt, solvate, hydrate or stereoisomer thereof; (2) a second therapeutic agent selected from the group consisting of: insulin sensitizers, sulfonylureas, biguanides, thiazolidinediones, α-glucosidase inhibitors, insulin secretogogues, insulin, antihyperlipidemic agents, plasma HDL-raising agents, HMG-CoA reductase inhibitors, statins, acryl CoA:cholestrol acyltransferase inhibitors, antiobesity compounds, antihypercholesterolemic agents, fibrates, vitamins and aspirin; and (3) optionally a pharmaceutically acceptable carrier.

Also encompassed by the present invention is a method of modulating a peroxisome proliferator activated receptor (PPAR), comprising the step of contacting the receptor with a compound of the present invention, or a pharmaceutically acceptable salt.

The method as recited above, wherein the peroxisome proliferator activated receptor is an alpha-receptor.

The method as recited above, wherein the peroxisome proliferator activated receptor is a gamma-receptor.

Also encompassed by the present invention is a method for treating or preventing a peroxisome proliferator activated receptor-gamma mediated disease or condition comprising the step of administering an effective amount of a compound of the present invention.

Also encompassed by the present invention is a method for lowering blood-glucose comprising the step of administering an effective amount of a compound of the present invention.

Also encompassed by the present invention is a method of treating or preventing disease or condition selected from the group consisting of hyperglycemia, dyslipidemia, Type II diabetes, Type I diabetes, hypertriglyceridemia, syndrome X, insulin resistance, heart failure, diabetic dyslipidemia, hyperlipidemia, hypercholesteremia, hypertension, obesity, anorexia bulimia, anorexia nervosa, cardiovascular disease and other diseases where insulin resistance is a component, comprising the step of administering an effective amount of a compound of the present invention.

Also encompassed by the present invention is a method of treating or preventing diabetes mellitus in a mammal comprising the step of administering to a mammal a therapeutically effective amount of a compound of the present invention.

Also encompassed by the present invention is a method of treating or preventing cardiovascular disease in a mammal comprising the step of administering to a mammal a therapeutically effective amount of a compound of the present invention, or a pharmaceutically acceptable salt or stereoisomer thereof.

Also encompassed by the present invention is a method of treating or preventing syndrome X in a mammal, comprising the step of administering to the mammal a therapeutically effective amount of a compound of the present invention, or a pharmaceutically acceptable salt or stereoisomer thereof.

Also encompassed by the present invention is a method of treating or preventing disease or condition selected from the group consisting of hyperglycemia, dyslipidemia, Type II diabetes, Type I diabetes, hypertriglyceridemia, syndrome X, insulin resistance, heart failure, diabetic dyslipidemia, hyperlipidemia, hypercholesteremia, hypertension, obesity, anorexia bulimia, anorexia nervosa, cardiovascular disease and other diseases where insulin resistance is a component, comprising the step of administering an effective amount of a compound of the present invention and an effective amount of second therapeutic agent selected from the group consisting of: insulin sensitizers, sulfonylureas, biguanides, thiazolidinediones, α-glucosidase inhibitors, insulin secretogogues, insulin, antihyperlipidemic agents, plasma HDL-raising agents, HMG-CoA reductase inhibitors, statins, acryl CoA:cholestrol acyltransferase inhibitors, antiobesity compounds, antihypercholesterolemic agents, fibrates, vitamins and aspirin.

Also encompassed by the present invention is use of a compound of the present invention or pharmaceutically acceptable salt or stereoisomer thereof for the manufacture of a medicament for the treatment of a condition modulated by a PPAR.

The terms used to describe the instant invention have the following meanings.

The term “alkyl,” unless otherwise indicated, refers to those alkyl groups having one to 14 carbon atoms, preferably one to six carbon atoms, of either a straight or branched saturated configuration including substituted alkyl. Examples of “alkyl” include, but are not limited to: methyl, ethyl, n-propyl, isopropyl, n-butyl, isobutyl, sec-butyl and tert-butyl, pentyl, hexyl, isopentyl and the like. Alkyl as defined above may be optionally substituted with a designated number of substituents as set forth in the embodiment recited above.

The term “alkynyl” means hydrocarbon chain of a specified number of carbon atoms (typically two to six carbon atoms) of either a straight or branched configuration and having at least one carbon-carbon triple bond, which may occur at any point along the chain. Example of alkynyl is acetylene. Alkynyl as defined above may be optionally substituted with designated number of substituents as set forth in the embodiment recited above.

The term “cycloalkyl” refers to a saturated or partially saturated carbocycle containing one or more rings having 3 to 12 carbon atoms, more typically 3 to 6 carbon atoms. Examples of cycloalkyl includes, but are not limited to cyclopropyl, cyclobutyl, cyclopentyl, cyclohexyl and cycloheptyl, and the like. Cycloalkyl as defined above may also includes a tricycle, such as adamantyl. Cycloalkyl as defined above may be optionally substituted with a designated number of substituents as set forth in the embodiment recited above.

The term “alkoxy” represents an alkyl group of indicated number of carbon atoms, typically one to six carbon atoms, attached through an oxygen bridge, such as methoxy, ethoxy, propoxy, isopropoxy, butoxy, tert-butoxy, pentoxy, and the like. Alkoxy as defined above may be optionally substituted with a designated number of substituents as set forth in the embodiment recited above.

The term “halo” refers to fluoro, chloro, bromo and iodo.

The term “haloalkyl” is a C₁-C₆ alkyl group, which is substituted with one or more halo atoms selected from F, Br, Cl and I. Examples of haloalkyl group are trifluoromethyl, CCl₃, CH₂CF₃ CH₂CCl₃ and the like.

The term “haloalkyloxy” represents a C₁-C₆ haloalkyl group attached through an oxygen bridge, such as OCF₃. The “haloalkyloxy” as defined above may be optionally substituted with a designated number of substituents as set forth in the embodiment recited above.

The term “aryl” includes carbocyclic aromatic ring systems (e.g. phenyl), fused polycyclic aromatic ring systems (e.g. naphthyl and anthracenyl) and aromatic ring systems fused to carbocyclic non-aromatic ring systems (e.g., 1,2,3,4-tetrahydronaphthyl). “Aryl” as defined above may be optionally substituted with a designated number of substituents as set forth in the embodiment recited above. Aryl defined above may also include aryl substituted with another aryl, for example biphenyl.

The term “aryloxy” represents an aryl group attached through an oxygen bridge, such as phenoxy (—O-phenyl). The “aryloxy” as defined above may be optionally substituted with a designated number of substituents as set forth in the embodiment recited above.

The term “acyl” refers to alkyl-C(═O)— group. Preferred acyl groups are those in which the alkyl group is (C₁-C₆)alkyl.

The term “heteroaryl” group, as used herein, is an aromatic ring system having at least one heteroatom such as nitrogen, sulfur or oxygen and includes monocyclic, bicyclic or tricyclic aromatic ring of 5- to 14-carbon atoms containing one or more heteroatoms selected from O, N, or S. The heteroaryl as defined above also includes heteroaryl fused with another heteroaryl, aryl fused with heteroaryl or aryl fused with heterocyclyl as defined herein. The “heteroaryl” may also be optionally substituted with a designated number of substituents as set forth in the embodiment recited above. Examples of heteroaryl are, but are not limited to: furanyl, thienyl (also referred to as “thiophenyl”), thiazolyl, imidazolyl, indolyl, isoindolyl, isooxazolyl, oxazoyl, pyrazolyl, pyrrolyl, pyrazinyl, pyridyl, pyrimidyl, pyrimidinyl and purinyl, cinnolinyl, benzofuranyl, benzothienyl (or benzothiophenyl), benzotriazolyl, benzoxazolyl, quinoline, isoxazolyl, isoquinoline 1,4 benzodioxan, or 2,3-dihydrobenzofuranyl and the like.

The term “heterocyclyl” refers to a non-aromatic ring which contains one or more heteroatoms selected from O, N or S, which includes a monocyclic, bicyclic or tricyclic ring of 5- to 14-carbon atoms containing one or more heteroatoms selected from O, N or S. The “heterocyclyl” as defined above may be optionally substituted with a designated number of substituents as set forth in the embodiment recited above. Examples of heterocyclyl include, but are not limited to, morpholine, piperidine, piperazine, pyrrolidine, and thiomorpholine.

An “arylalkyl” as used herein is an aryl substituent that is linked to a compound by an alkyl group having from one to six carbon atoms. The “arylalkyl” as defined above may be optionally substituted with a designated number of substituents as set forth in the embodiment recited above.

An aminoalkyl group is an alkyl group having from one to six carbon atoms, which is substituted with at least one amine represented by NR¹²R¹² where each R¹² is independently a C₁-C₆ alkyl or both R¹² taken together with the nitrogen to which they are attached form a five or six membered heterocycloalkyl.

The term R^{5A} (or bioisosteres) as used herein includes, but are not limited to, carboxamide, sulfonamide, acylsulfonamide, tetrazole or the following moiety.

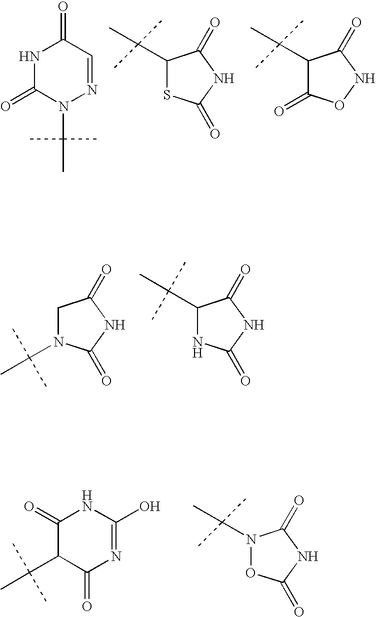

Carboxamide, sulfonamide, acylsulfonamide and tetrazole may be optionally substituted with one or more suitable substituents selected from haloalkyl, aryl, heteroaryl, and C₁-C₆ alkyl. The heteroalkyl, aryl, heteroaryl and alkyl may further optionally substituted with one or more substituents selected from the list provided for R⁶ or R^{6a}. The examples of R^{5A} are, but not limited to, hydroxamic acid, acyl cyanamide, tetrazoles, sulfinylazole, sulfonylazole, 3-hydroxyisoxazole, hydroxythiadiazole, sulphonate and acylsulfonamide.

The term “active ingredient” means the compounds generically described by formula I as well as the salts, solvates and prodrugs of such compounds.

The term “pharmaceutically acceptable” means that the carrier, diluents, excipients and salt must be compatible with the other ingredients of the composition, and not deleterious to the recipient thereof. Pharmaceutical compositions of the present invention are prepared by procedures known in the art using well-known and readily available ingredients.

“Preventing” refers to reducing the likelihood that the recipient will incur or develop any of the pathological conditions described herein.

“Treating” refers to mediating a disease or condition, and preventing or mitigating its further progression or ameliorates the symptoms associated with the disease or condition.

“Pharmaceutically-effective amount” means that amount of a compound of the present invention, or of its salt, solvate, hydrate or prodrug thereof that will elicit the biological or medical response of a tissue, system or mammal. Such an amount can be administered prophylactically to a patient thought to be susceptible to development of a disease or condition. Such amount when administered prophylactically to a patient can also be effective to prevent or lessen the severity of the mediated condition. Such an amount is intended to include an amount, which is sufficient to modulate a PPAR receptor such as a PPARα or PPARγ receptor to mediate a disease or condition. Conditions mediated by PPARα or PPARγ receptors include, for example, diabetes mellitus, cardiovascular disease, Syndrome X, obesity and gastrointestinal disease. Additional conditions associated with the modulation of a PPAR receptor include inflammation related conditions which include, for example, IBD (inflammatory bowel disease), rheumatoid arthritis, psoriasis, Alzheimer's disease, Chrohn's disease and ischemia reprofusion injury (stroke and miocardial infarction).

A “mammal” is an individual animal that is a member of the taxonomic class Mammalia. The class Mammalia includes humans, monkeys, chimpanzees, gorillas, cattle, swine, horses, sheep, dogs, cats, mice, rats and the like.

Administration to a human is most preferred. A human to whom the compounds and compositions of the present invention are administered has a disease or condition in which control blood glucose levels are not adequately controlled without medical intervention, but wherein there is endogenous insulin present in the human's blood. Non-insulin dependent diabetes mellitus (NIDDM) is a chronic disease or condition characterized by the presence of insulin in the blood, even at levels above normal, but resistance or lack of sensitivity to insulin action at the tissues.

Those skilled in the art will recognize that sterocenters exist in compound of Formula I. Accordingly, the present invention includes all possible stereoisomers and geometric isomers of formula I including racemic compounds and the optically active isomers.

The compounds of the present invention contain one or more chiral centers and exist in different optically active forms. When compounds of formula I contain one chiral center, the compounds exist in two enantiomeric forms and the present invention includes both enantiomers and mixtures of enantiomers, such as racemic mixtures. Resolution of the final product, an intermediate or a starting material may be effected by any suitable method known in the art, for example by formation of diastereoisomeric salts which may be separated by crystallization; formation of diastereoisomeric derivatives or complexes which may be separated by crystallization and gas-liquid or liquid chromatography; selective reaction of one enantiomer with an enantiomer-specific reagent such as enzymatic esterification; and gas-liquid or liquid chromatography in a chiral environment such as on a chiral support, for example silica with a bound chiral ligand or in the presence of a chiral solvent. See also Sterochemisty of Carbon Compounds by E. L. Eliel (Mcgraw Hill, 1962) and Tables of Resolving Agents by S. H. Wilen. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation. In a more preferred embodiment, the compounds of the present invention are S-enantiomers.

When a compound of formula I has more than one chiral substituents, it may exist in diastereoisomeric forms. The diastereoisomeric pairs may be separated by methods known to those skilled in the art, for example chromatography or crystallization and the individual enantiomers within each pair may be separated as described above. The present invention includes each diastereoisomer of compounds of formula I and mixtures thereof.

Certain compounds of the present invention may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of formula I and Ia and mixtures thereof.

Certain compound of the present invention may exist in zwitterionic form, and the present invention includes each zwitterionic form of compounds of formula I and mixtures thereof.

Certain compounds of the present invention and their salts may exist in more than one crystal form. Polymorphs of compounds of formula I form part of the present invention and may be prepared by crystallization of a compound of formula I under different conditions, such as using different solvents or different solvent mixtures for recrystallization; crystallization at different temperatures; and various modes of cooling ranging from very fast to very slow cooling during crystallization. Polymorphs may also be obtained by heating or melting a compound of formula I followed by gradual or fast cooling. The presence of polymorphs may be determined by solid probe NMR spectroscopy, IR spectroscopy, differential scanning calorimetry, powder X-ray diffraction or other available techniques.

Certain compounds of the present invention and their salts may exist in more than one crystal form, and the present invention includes each crystal form and mixtures thereof.

Certain compounds of the present invention and their salts may also exist in the form of solvates, for example hydrates, and the present invention includes each solvate and mixtures thereof.

“Pharmaceutically-acceptable salt” refers to salts of the compounds of formula I, which are substantially non-toxic to mammals. Typical pharmaceutically acceptable salts include those salts prepared by reaction of the compounds of the present invention with a mineral, organic acid: an organic base or inorganic base. Such salts are known as base addition salts, respectively. It should be recognized that the particular counterion forming a part of any salt of the present invention is not of a critical nature so long as the salt as a whole is pharmaceutically acceptable and the counterion does not contribute undesired qualities to the salt as a whole.

By virtue of its acidic moiety, a compound of formula I salts with pharmaceutically acceptable bases. Some examples of base addition salts include metal salts such as aluminum; alkali metal salts such as lithium, sodium or potassium; and alkaline earth metal salts such as calcium, magnesium, ammonium, or substituted ammonium salts. Examples of substituted ammonium salts include, for instance, those with lower alkylamines such as trimethylamine and triethylamine; hydroxyalkylamines such as 2-hydroxyethylamine, bis-(2-hydroxyethyl)-amine or tri-(2-hydroxyethyl)-amine; cycloalkylamines such as bicyclohexylamine or dibenzylpiperidine, N-benzyl-β-phenethylamine, dehydroabietylamine, N,N′-bisdehydro-abietylamine, glucamine, N-piperazine methylglucamine; bases of the pyridine type such as pyridine, collidine, quinine or quinoline; and salts of basic amino acids such as lysine and arginine.

Examples of inorganic bases include, without limitation, sodium hydroxide, potassium hydroxide, potassium carbonate, sodium carbonate, sodium bicarbonate, potassium bicarbonate, calcium hydroxide, calcium carbonate, and the like.

Compounds of the present invention which are substituted with a basic group, may exist as salts with pharmaceutically acceptable acids. The present invention includes such salts. Examples of such salts include hydrochlorides, hydrobromides, sulfates, methanesulfonates, nitrates, maleates, acetates, citrates, fumarates, tartrates [e.g. (+)-tartrates, (−)-tartrates or mixtures thereof including racemic mixtures], succinates, benzoates and salts with amino acids such as glutamic acid. These salts may be prepared by methods known to those skilled in the art.

The compounds of the present invention (or salt) may also form a solvate with water (e.g., hydrate) or an organic solvent, and the present invention encompasses any solvate, hydrate or any mixtures thereof.

The compounds of the present invention, which bind to and activate the PPARs, lower one or more of glucose, insulin, triglycerides, fatty acids and/or cholesterol, and are therefore useful for the treatment and/or prevention of hyperglycemia, dyslipidemia and in particular Type II diabetes as well as other diseases including syndrome X, Type I diabetes, hypertriglyceridemia, insulin resistance, diabetic dyslipidemia, hyperlipidemia, hypercholesteremia, heart failure, coagaulopathy, hypertension, and cardiovascular diseases, especially arteriosclerosis. In addition, these compounds are indicated to be useful for the regulation of appetite and food intake in subjects suffering from disorders such as obesity, anorexia bulimia and anorexia nervosa.

The compounds and compositions of the present invention are also useful to treat acute or transient disorders in insulin sensitivity, which sometimes occurs following a surgery, trauma, myocardial infarction and the like. The compounds and compositions of the present invention are also useful for lowering serum triglyceride levels. Elevated triglyceride level, whether caused by genetic predisposition or by a high fat diet, is a risk factor for the development of heart disease, stroke, and circulatory system disorders and diseases. The physician of ordinary skill will know how to identify humans who can benefit from administration of the compounds and compositions of the present invention.

The present invention further provides a method for the treatment and/or prophylaxis of hyperglycemia in a human or non-human mammal which comprises administering an effective, non-toxic amount of a compound of formula I, or a tautomeric form thereof and/or a pharmaceutically acceptable salt thereof and/or a pharmaceutically acceptable solvate thereof to a hyperglycemic human or non-human mammal in need thereof.

The compounds of the present invention are useful as therapeutic substances in preventing or treating Syndrome X, diabetes mellitus and related endocrine and cardiovascular disorders and diseases in human or non-human animals.

The present invention also relates to the use of a compound of formula I as described above for the manufacture of a medicament for treating a PPARα or PPARγ mediated condition, separately or in combination.

A therapeutically effective amount of a compound of formula I can be used for the preparation of a medicament useful for treating Syndrome X, diabetes, treating obesity, lowering triglyceride levels, raising the plasma level of high density lipoprotein, and for treating, preventing or reducing the risk of developing arteriosclerosis, and for preventing or reducing the risk of having a first or subsequent atherosclerotic disease event in mammals, particularly in humans. In general, a therapeutically effective amount of a compound of formula I of the present invention typically reduces serum glucose levels, more specifically HbA1c, of a patient by about 0.7% or more; typically reduces serum triglyceride levels of a patient by about 20% or more; and increases serum HDL levels in a patient. Preferably, HDL levels can be increased by about 30% or more.

Additionally, an effective amount of a compound of formula I and a therapeutically effective amount of one or more active agents selected from antihyperlipidemic agent, plasma HDL-raising agents, antihypercholesterolemic agents, fibrates, vitamins, aspirin, insulin secretogogues, insulin and the like can be used together for the preparation of a medicament useful for the above described treatments.

Advantageously, compositions containing the compound of formula I or the salts thereof may be provided in dosage unit form, preferably each dosage unit containing from about 1 to about 500 mg. It is understood that the amount of the compounds or compounds of formula I that will be administered is determined by a physician considering of all the relevant circumstances.

Syndrome X includes pre-diabetic insulin resistance syndrome and the resulting complications thereof, insulin resistance, non-insulin dependent diabetes, dyslipidemia, hyperglycemia obesity, coagulopathy, hypertension and other complications associated with diabetes. The methods and treatments mentioned herein include the above and encompass the treatment and/or prophylaxis of any one of or any combination of the following: pre-diabetic insulin resistance syndrome, the resulting complications thereof, insulin resistance, Type II or non-insulin dependent diabetes, dyslipidemia, hyperglycemia, obesity and the complications associated with diabetes including cardiovascular disease, especially arteriosclerosis.

The compositions are formulated and administered in the same general manner as detailed herein. The compounds of the present invention may be used effectively alone or in combination with one or more additional active agents depending on the desired target therapy. Combination therapy includes administration of a single pharmaceutical dosage composition, which contains a compound of formula I and one or more additional active agents, as well as administration of a compound of formula I and each active agent in its own separate pharmaceutical dosage formulation. For example, a compound of formula I or thereof and an insulin secretogogue such as biguanides, thiazolidinediones, sulfonylureas, insulin or α-glucosidose inhibitors can be administered to the patient together in a single oral dosage composition such as a tablet or capsule, or each agent administered in separate oral dosage formulations. Where separate dosage formulations are used, a compound of formula I and one or more additional active agents can be administered at essentially the same time, i.e., concurrently or at separately staggered times, i.e., sequentially; combination therapy is understood to include all these regimens.

An example of combination treatment or prevention of arteriosclerosis may involve administration of a compound of formula I or salts thereof in combination with one or more of second active therapeutic agents: antihyperlipidemic agents; plasma HDL-raising agents; antihypercholesterolemic agents, fibrates, vitamins, aspirin and the like. As noted above, the compounds of formula I can be administered in combination with more than one additional active agent.

Another example of combination therapy can be seen in treating diabetes and related disorders wherein the compounds of formula I or salts thereof can be effectively used in combination with second active therapeutic, such as sulfonylureas, biguanides, thiazolidinediones, α-glucosidase inhibitors, other insulin secretogogues, insulin as well as the active agents discussed above for treating arteriosclerosis.

The examples of second therapeutic agents are insulin sensitizers, PPARα agonists, glitazones, troglitazone, pioglitazone, englitazone, MCC-555, BRL 49653, biguanides, metformin, phenformin, insulin, insulin minetics, sulfonylureas, tolbutamide, glipizide, alpha-glucosidase inhibitors, acarbose, cholesterol lowering agent, HMG-CoA reductase inhibitors, lovastatin, simvastatin, pravastatin, fluvastatin, atrovastatin, rivastatin, other statins, sequestrates, cholestyramine, colestipol, dialkylaminoalkyl derivatives of a cross-linked dextran, nicotinyl alcohol, nicotinic acid: a nicotinic acid salt, PPARα agonists, fenofibric acid derivatives, gemfibrozil, clofibrate, fenofibrate, benzafibrate, inhibitors of cholesterol absorption, beta-sitosterol, acryl CoA:cholesterol acyltransferase inhibitors, melinamide, probucol, PPARδ agonists, antiobesity compounds, fenfluramine, dexfenfluramine, phentiramine, sulbitramine, orlistat, neuropeptide Y5 inhibitors, β₃ adrenergic receptor agonists, and ileal bile acid transporter inhibitors.

The compounds of the present invention and the pharmaceutically acceptable salts, solvates and hydrates thereof have valuable pharmacological properties and can be used in pharmaceutical compositions containing a therapeutically effective amount of a compound of the present invention, or pharmaceutically acceptable salts, esters or prodrugs thereof, in combination with one or more pharmaceutically acceptable excipients. Excipients are inert substances such as, without limitation carriers, diluents, fillers, flavoring agents, sweeteners, lubricants, solubilizers, suspending agents, wetting agents, binders, disintegrating agents, encapsulating material and other conventional adjuvants. Proper formulation is dependent upon the route of administration chosen. Pharmaceutical compositions typically contain from about 1 to about 99 weight percent of the active ingredient, which is a compound of the present invention.

Preferably, the pharmaceutical formulation is in unit dosage form. A “unit dosage form” is a physically discrete unit containing a unit dose suitable for administration in human subjects or other mammals. For example, a unit dosage form can be a capsule or tablet, or a number of capsules or tablets. A “unit dose” is a predetermined quantity of the active compound of the present invention, calculated to produce the desired therapeutic effect, in association with one or more pharmaceutically acceptable excipients. The quantity of active ingredient in a unit dose may be varied or adjusted from about 0.1 to about 1000 milligrams or more according to the particular treatment involved.

The dosage regimen utilizing the compounds of the present invention is selected by one of ordinary skill in the medical or veterinary arts considering various factors, such as without limitation, the species, age, weight, sex, medical condition of the recipient, the severity of the condition to be treated, the route of administration, the level of metabolic and excretory function of the recipient, the dosage form employed, the particular compound and salt thereof employed, and the like.

Preferably, the compounds of the present invention are administered in a single daily dose, or the total daily dose may be administered in divided doses of two, three or more times per day. Where delivery is via transdermal forms, administration is continuous.

Suitable routes of administration of pharmaceutical compositions of the present invention include, for example, oral, eye drop, rectal, transmucosal, topical or intestinal administration; parenteral delivery (bolus or infusion), including intramuscular, subcutaneous, intramedullary injections, as well as intrathecal, direct intraven-tricular, intravenous, intraperitoneal, intranasal, or intraocular injections. The compounds of the present invention can also be administered in a targeted drug delivery system, such as in a liposome coated with endothelial cell-specific antibody.

For oral administration, the compounds of the present invention can be formulated readily by combining the active compounds with pharmaceutically acceptable carriers well known in the art. Such carriers enable the compounds of the present invention to be formulated as tablets, pills, powders, sachets, granules, dragees, capsules, liquids, elixirs, tinctures, gels, emulsions, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores.

For oral administration in the form of a tablet or capsule, the active ingredient may be combined with an oral, non-toxic, pharmaceutically-acceptable carrier, such as, without limitation, lactose, starch, sucrose, glucose, methyl cellulose, calcium carbonate, calcium phosphate, calcium sulfate, sodium carbonate, mannitol, sorbitol, and the like; together with, optionally, disintegrating agents, such as, without limitation, cross-linked polyvinyl pyrrolidone, maize, starch, methyl cellulose, agar, bentonite, xanthan gum, alginic acid: or a salt thereof such as sodium alginate, and the like; and, optionally, binding agents, for example, without limitation, gelatin, acacia, natural sugars, beta-lactose, corn sweeteners, natural and synthetic gums, acacia, tragacanth, sodium alginate, carboxymethyl-cellulose, polyethylene glycol, waxes, and the like; and, optionally, lubricating agents, for example, without limitation, magnesium stearate, sodium stearate, stearic acid: sodium oleate, sodium benzoate, sodium acetate, sodium chloride, talc, and the like. When a dosage unit form is a capsule, it may contain, in addition to materials of the above type, a liquid carrier such as a fatty oil.

Solid form formulations include powders, tablets and capsules. A solid carrier can be one or more substances, which may also act as flavoring agents, lubricants, solubilisers, suspending agents, binders, tablet disintegrating agents and encapsulating material.

In powders, the carrier is a finely divided solid which is in admixture with the finely divided active ingredient. In tablets, the active ingredient is mixed with a carrier having the necessary binding properties in suitable proportions and compacted in the shape and size desired.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Sterile liquid formulations include suspensions, emulsions, syrups, and elixirs. The active ingredient can be dissolved or suspended in a pharmaceutically acceptable carrier, such as sterile water, sterile organic solvent, or a mixture of both sterile water and sterile organic solvent.

The active ingredient can also be dissolved in a suitable organic solvent, for example, aqueous propylene glycol. Other compositions can be made by dispersing the finely divided active ingredient in aqueous starch or sodium carboxymethyl cellulose solution or in a suitable oil.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

All formulations for oral administration should be in dosages suitable for such administration. Particularly suitable compositions for oral administration are unit dosage forms such as tablets and capsules.

For parental administration the compounds of the present invention or salts thereof can be combined with sterile aqueous or organic media to form injectable solutions or suspensions. Formulations for injection may be presented in unit dosage form, such as in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that each syringability exists. It must be stable under the conditions of manufacture and storage and must be preserved against any contamination. The carrier can be solvent or dispersion medium containing, for example, water, preferably in physiologically compatible buffers such as Hanks's solution, Ringer's solution, or physiological saline buffer, ethanol, polyol (e.g. glycerol, propylene glycol and liquid polyethylene glycol), propylene glycol and liquid polyethylene glycol), suitable mixtures thereof, and vegetable oils. Under ordinary conditions of storage and use, these preparations contain a preservative to prevent the growth of microorganisms.

The injectable solutions prepared in this manner can then be administered intravenously, intraperitoneally, subcutaneously, or intramuscularly, with intramuscular administration being preferred in humans.

For transmucosal administration, penetrants appropriate to the barrier to be permeated are used in the formulation. Such penetrants are generally known in the art. The active compounds can also be administered intranasally as, for example, liquid drops or spray.

For buccal administration, the compositions may take the form of tablets or lozenges formulated in a conventional manner.

For administration by inhalation, the compounds for use according to the present invention are conveniently delivered in the form of a dry powder inhaler, or an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of gelatin for use in an inhaler or insufflator may be formulated containing a powder mix of the compound and a suitable powder base such as lactose or starch.

Pharmaceutical compositions of the present invention can be manufactured in a manner that is itself known, e.g., by means of conventional mixing, dissolving, granulating, dragee-making, levigating, emulsifying, encapsulating, entrapping or lyophilizing processes.

In making the compositions of the present invention, the active ingredient will usually be admixed with a carrier, or diluted by a carrier, or enclosed within a carrier which may be in the form of a capsule, sachet, paper or other container. When the carrier serves as a diluent, it may be a solid, lyophilized solid or paste, semi-solid, or liquid material which acts as a vehicle, or can be in the form of tablets, pills, powders, lozenges, elixirs, suspensions, emulsions, solutions, syrups, aerosols (as a solid or in a liquid medium), or ointment, containing for example up to 10% by weight of the active compound. The compounds of the present invention are preferably formulated prior to administration.

Binding and Cotransfection Studies

The in vitro potency of compounds in modulating PPARγ, PPARα and PPARδ receptors are determined by the procedures detailed below. DNA-dependent binding (ABCD binding) is carried out using Scintillation Proximity Assay (SPA) technology with PPAR receptors. Tritium-labeled PPARα and PPARγ agonists are used as radioligands for generating displacement curves and IC₅₀ values with compounds of the present invention. Cotransfection assays are carried out in CV-1 cells. The reporter plasmid contains an acylCoA oxidase (AOX) PPRE and TK promoter upstream of the luciferase reporter cDNA. Appropriate PPARs and RXRα are constitutively expressed using plasmids containing the CMV promoter. Since for PPARα and PPARβ, interference by endogenous PPARγ in CV-1 cells is an issue, in order to eliminate such interference, a GAL4 chimeric system is used in which the DNA binding domain of the transfected PPAR is replaced by that of GAL4, and the GAL4 response element is utilized in place of the AOX PPRE. Cotransfection efficacy is determined relative to PPARα agonist and PPARγ agonist reference molecules. Efficacies are determined by computer fit to a concentration-response curve, or in some cases at a single high concentration of agonist (10 μM). A typical range for concentration determination (IC₅₀) is from 1 nM to 10 μM. For binding or cotransfection studies with receptors other than PPARs, similar assays are carried out using appropriate ligands, receptors, reporter constructs and etc. for that particular receptor.

These studies are carried out to evaluate the ability of compounds of the present invention to bind to and/or activate various nuclear transcription factors, particularly huPPARα (“hu” indicates “human”), huPPARγ and huPPARδ. These studies provide in-vitro data concerning efficacy and selectivity of compounds of the present invention. Furthermore, binding and cotransfection data for compounds of the present invention are compared with corresponding data for reference compounds that act on either huPPARα or huPPARγ. The typical range of concentration for binding of the compounds is from 1 nM to 10 μM. The concentration of test compound required to effect 50% maximal activation of PPARα (IC₅₀) and PPARγ (IC₅₀γ) is determined.

The compounds of the present invention are found to have IC₅₀ or EC₅₀ in the range of 1 nM to 10 μM, preferably 1 nM to 1000 nM for PPAR gamma.

### Evaluation of Triglyceride and Cholesterol Level in HuapoAI Transgenic Mice

Five to six week old male mice, transgenic for human apoAI [C57B1/6-tgn(apoa1)1rub, Jackson Laboratory, Bar Harbor, Me.] are housed five per cage (10″×20″×8″ with aspen chip bedding) with food (Purina 5001) and water available at all times. After an acclimation period of 2 weeks, animals are individually identified by ear notches, weighed and assigned to groups based on body weight. Beginning the following morning, mice are dosed daily by oral gavage for 7 days using a 20 gauge, 1½″ curved disposable feeding needle. Treatments are test compounds (30 mg/kg), a positive control (fenofibrate, 100 mg/kg) or vehicle [1% carboxymethylcellulose (w/v)/0.25% Tween80 (w/v); 0.2 ml/mouse]. Prior to termination on day 7, mice are weighed and dosed. Three hours after dosing, animals are anesthetized by inhalation of isoflurane (2-4%) and blood obtained via cardiac puncture (0.7-1.0 ml). Whole blood is transferred to serum separator tubes (Vacutainer SST), chilled on ice and permitted to clot. Serum is obtained after centrifugation at 4° C. and frozen until analysis for triglycerides, total cholesterol, compound levels and serum lipoprotein profile by fast protein liquid chromatography (FPLC) coupled to an inline detection system. After sacrifice by cervical dislocation, the liver, heart and epididymal fat pads are excised and weighed.

The animals dosed with vehicle have average triglycerides values of about 60 to 80 mg/dl, which are reduced by the positive control fenofibrate (33-58 mg/dl with a mean reduction of 37%). The animals dosed with vehicle have average total serum cholesterol values of about 140 to 180 mg/dl, which are increased by fenofibrate (about 190 to 280 mg/dl with a mean elevation of 41%). When subject to FPLC analysis, pooled sera from vehicle-treated hu apoAI transgenic mice have a high-density lipoprotein cholesterol (HDLc) peak area which ranges from 47v-sec to 62v-sec. Fenofibrate increases the amount of HDLc (68-96v-sec with a mean percent increase of 48%). Test compounds evaluated in terms of percent increase in the area under the curve. Representative compounds of the present invention are tested using the above methods or substantially similar methods.

Evaluation of Glucose Levels in db/db Mice

Five week old male diabetic (db/db) mice [C57B1Ks/j-m+/+Lepr(db), Jackson Laboratory, Bar Harbor, Me.] or lean littermates (db+) are housed 6 per cage (10″×20″×8″ with aspen chip bedding) with food (Purina 5015) and water available at all times. After an acclimation period of 2 weeks, animals are individually identified by ear notches, weighed and bled via the tail vein for determination of initial glucose levels. Blood is collected (100 μl) from unfasted animals by wrapping each mouse in a towel, cutting the tip of the tail with a scalpel, and milking blood from the tail into a heparinized capillary tube balanced on the edge of the bench. Sample is discharged into a heparinized microtainer with gel separator (VWR) and retained on ice. Plasma is obtained after centrifugation at 4° C. and glucose is measured immediately. Remaining plasma is frozen until the completion of the experiment, and glucose and triglycerides are assayed in all samples. Animals are grouped based on initial glucose levels and body weights. Beginning the following morning, mice are dosed daily by oral gavage for 7 days using a 20 gauge, 1½″ curved disposable feeding needle. Treatments are test compounds (30 mg/kg), a positive control agent (30 mg/kg) or vehicle [1% carboxymethylcellulose (w/v)/0.25% Tween80 (w/v); 0.3 ml/mouse]. On day 7, mice are weighed and bled (tail vein) for about 3 hours after dosing. Twenty-four hours after the 7^{th} dose (i.e., day 8), animals are bled again (tail vein). Samples obtained from conscious animals on days 0, 7 and 8 are assayed for glucose. After 24 hour bleed, animals are weighed and dosed for the final time. Three hours after dosing on day 8, animals are anesthetized by inhalation of isoflurane, and blood obtained is via cardiac puncture (0.5-0.7 ml). Whole blood is transferred to serum separator tubes, chilled on ice and permitted to clot. Serum is obtained after centrifugation at 4° C. and frozen until analysis for compound levels. After sacrifice by cervical dislocation, the liver, heart and epididymal fat pads are excised and weighed.

The animals dosed with vehicle have average triglycerides values of about 170 to 230 mg/dl, which are reduced by the positive PPARγ control (about 70 to 120 mg/dl with a mean reduction of 50%). Male db/db mice are hyperglycemic (average glucose of about 680 to 730 mg/dl on the 7^{th} day of treatment), while lean animals have average glucose levels between about 190 and 230 mg/dl. Treatment with the positive control agent reduces glucose significantly (about 350 to 550 mg/dl with a mean decrease towards normalization of 56%).

Glucose is measured calorimetrically by using commercially purchased reagents (Sigma #315-500). According to the manufacturers, the procedures are modified from published work (McGowan et al. Clin Chem, 20:470-5 (1974) and Keston, A. Specific colorimetric enzymatic analytical reagents for glucose. Abstract of papers 129th Meeting ACS, 31C (1956).); and depend on the release of a mole of hydrogen peroxide for each mole of analyte coupled with a color reaction first described by Trinder (Trinder, P. Ann Clin Biochem, 6:24 (1969)). The absorbance of the dye produced is linearly related to the analyte in the sample. The assays are further modified for use in a 96 well format. Standards (Sigma #339-11, Sigma #16-11, and Sigma #CCO0534 for glucose, triglycerides and total cholesterol, respectively), quality control plasma (Sigma #A2034), and samples (2 or 5 μl/well) are measured in duplicate using 200 μl of reagent. An additional aliquot of sample, pipetted to a third well and diluted in 200 μl water, provided a blank for each specimen. Plates are incubated at room temperature (18, 15, and 10 minutes for glucose, triglycerides and total cholesterol, respectively) on a plate shaker and absorbance read at 500 nm (glucose and total cholesterol) or 540 nm (triglycerides) on a plate reader. Sample absorbance is compared to a standard curve (100-800, 10-500, and 100-400 mg/dl for glucose, triglycerides and total cholesterol, respectively). Values for the quality control sample are consistently within the expected range and the coefficient of variation for samples is below 10%. All samples from an experiment are assayed at the same time to minimize inter-assay variability.

Serum lipoproteins are separated and cholesterol is quantitated with an in-line detection system. Sample is applied to a Superose® 6 HR 10/30-size exclusion column (Amersham Pharmacia Biotech) and eluted with phosphate buffered saline-EDTA at 0.5 ml/min. Cholesterol reagent (Roche Diagnostics Chol/HP 704036) at 0.16 ml/min is mixed with the column effluent through a T-connection, and the mixture is passed through a 15 m×0.5 mm id knitted tubing reactor immersed in a 37° C. water bath. The colored product produced in the presence of cholesterol is monitored in the flow stream at 505 nm, and the analog voltage from the monitor is converted to a digital signal for collection and analysis. The change in voltage corresponding to change in cholesterol concentration is plotted against time, and the area under the curve corresponding to the elution of VLDL, LDL and HDL is calculated (Perkin Elmer Turbochrome software).

The compounds of the present invention can be prepared according to the procedures of the following schemes and examples, which may further illustrate details for the preparation of the compounds of the present invention. The compounds illustrated in the schemes and examples are, however, not to be construed as forming the only genus that is considered as the present invention.

The compounds of the present invention, in general, may be prepared according to the Reaction Schemes described below. When describing various aspects of the compounds disclosed herein, the terms “Tail” and “Head” are used as their concept illustrated below.

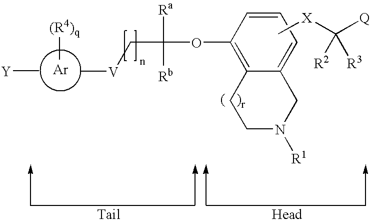

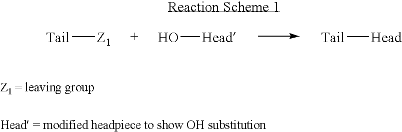

As shown in Reaction Scheme 1, the compounds of the present invention, in general, can be divided into Tail and Head regions where a nucleophilic headpiece coupled with an electrophilic tailpiece. These regions can be further modified as shown in the following reaction schemes.

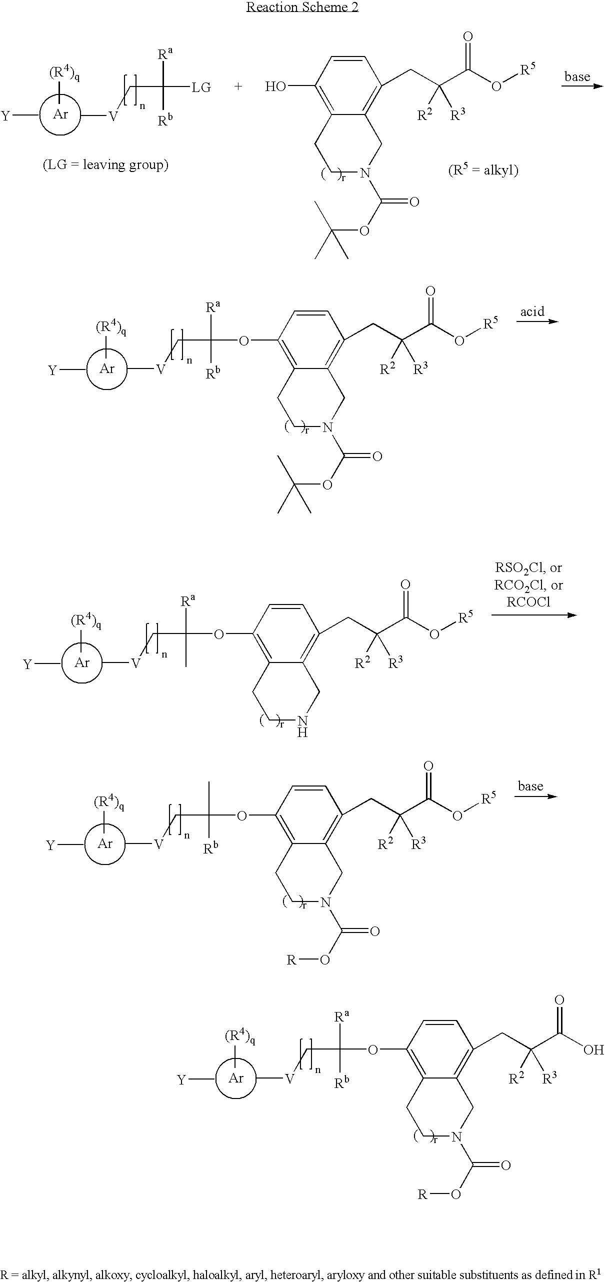

As shown in Reaction Scheme 2, an appropriately substituted tailpiece can be coupled with the desired headpiece using an appropriate base such as Cs₂CO₃ in a suitable solvent such as DMF, typically at 55° C. The nitrogen of the product can then be deprotected using an appropriate acid to give the required amine. This amine can then be coupled with a sulfonyl chloride, chloroformate, or acylchloride in the suitable solvent such as methylene chloride at 0° C. to give the desired sulfonated, carbamylated or acylated product. Finally the ester is converted to the acid using an appropriate base such as sodium hydroxide in a suitable solvent such as methanol, typically at 60° C. to give the final products.

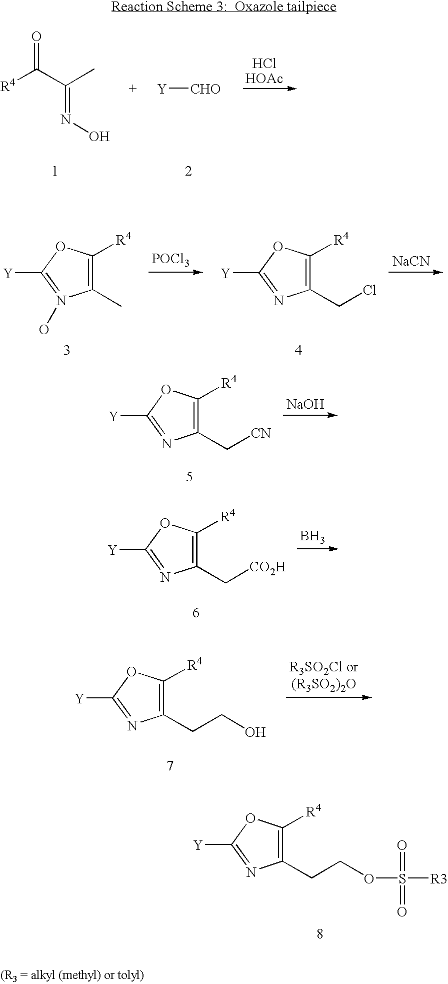

As shown in Reaction Scheme 3, an intermediate oxazole tailpiece can be prepared by a condensation of dionemonoxime (1) with aldehyde (2) such as bromobenzaldehyde in the presence of acid such as hydrochloric acid or acetic acid to give an oxazole n-oxide compound (3). The oxazole n-oxide is then treated with phosphorous oxychloride in an organic solvent to form chloromethyl substituted-oxazole (4). Compound (4) is further treated with a cyanide to form cyanomethyl oxazole compound (5). The cyano group of compound (5) is converted to a carboxylic acid group by treatment with an alkali metal hydroxide such as NaOH to form carboxymethyl substituted oxazole (6), which is further treated with a carboxylic acid reducing agent, such as borane or lithium aluminum hydride (LAH) to form compound (7). Compound (7) can be converted to oxazolyl sulfonyl ester (8) in the presence of a base by treatment with a sulfonyl halide or sulfonyl anhydride (R₃SO₂Cl or (R₃SO₂)₂O), such as tosyl anhydride, mesyl anhydride, tosyl chloride or mesyl chloride.

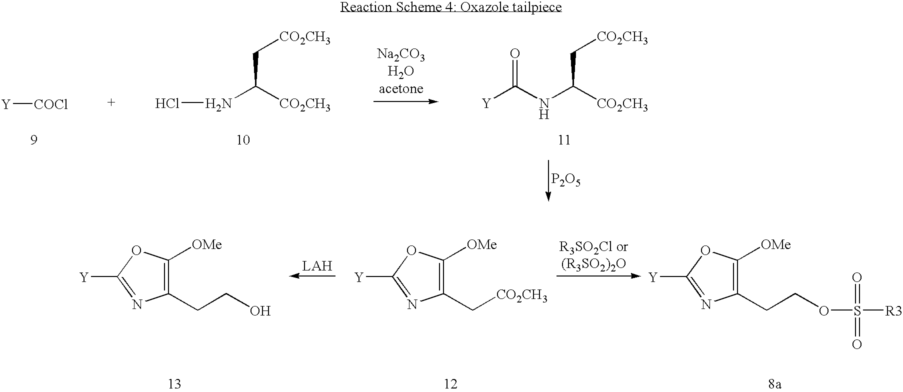

Alternatively, an intermediate of oxazole tailpiece can be prepared as shown in Reaction Scheme 4. Acid chloride (9) is reacted with L-aspartic acid dimethyl ester (10) to give amide compound (11), which undergoes cyclization to form an oxazole ring (12) by treatment with a dehydrating agent such as P₂O₅. The ester compound (12) is reduced by treating with LAH to give alcohol (13), which is then converted to oxazolyl sulfonyl ester (8a) as described above in Reaction Scheme 2.

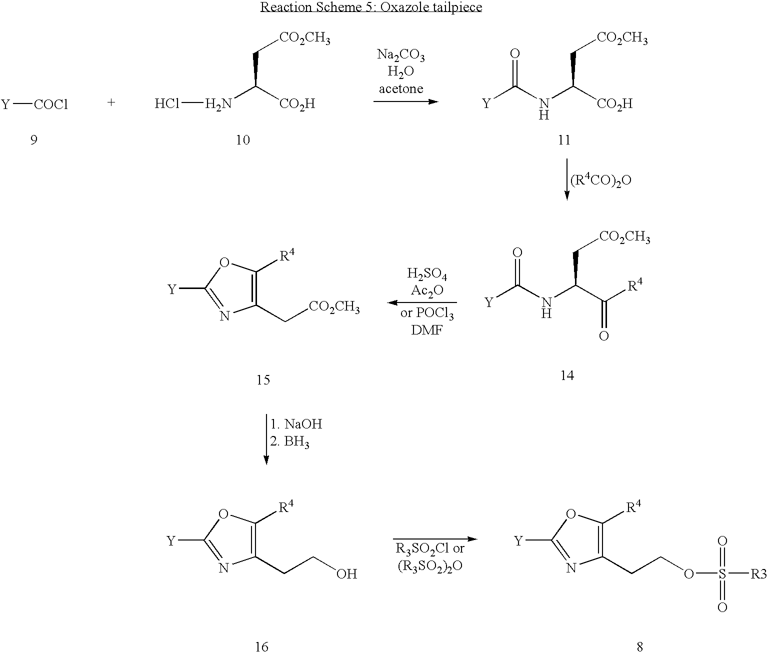

Another route to an intermediate of oxazole tailpiece is shown in Reaction Scheme 5. Acid chloride (9) and L-aspartic acid monomethyl ester (10) are reacted to give amide compound (11), which is further reacted to give ketone (14). The ketone compound undergoes a cyclization in the presence of dehydrating agent such as POCl₃ or H₂SO₄/acetic anhydride to form oxazole ring (15). Compound (15) undergoes reduction to give alcohol (16), which is then converted to oxazolyl sulfonyl ester (8) as described above in Reaction Scheme 3.

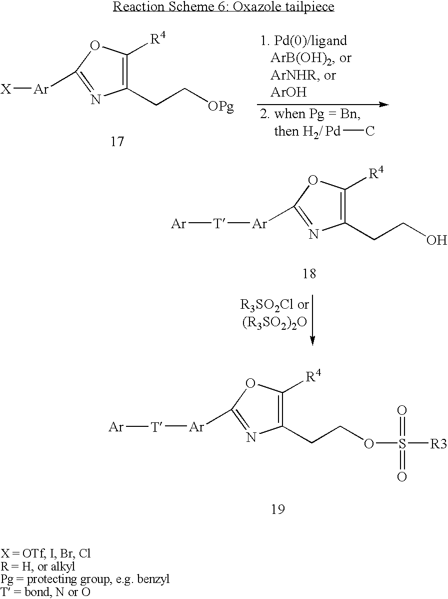

Another route to an intermediate of the oxazole tailpiece is shown in Reaction Scheme 6. The oxazole compound (17) can undergo a coupling reaction in the presence of palladium catalyst with an aryl boronic acid, aryl alcohol or aryl amine followed by deprotection to yield the corresponding compound (18). Compound (18) is then converted to oxazolyl sulfonyl ester (19) as described above in Reaction Scheme 3.

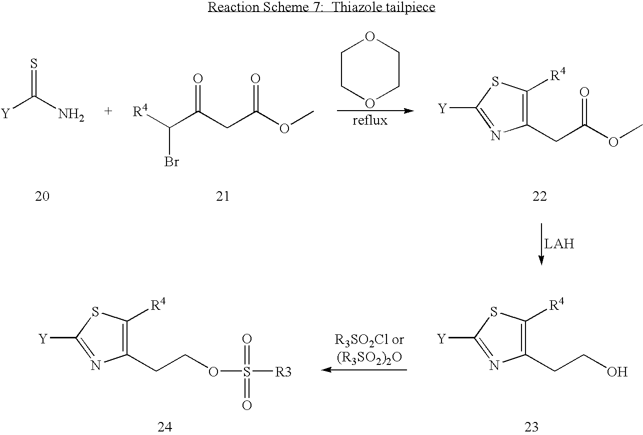

As shown in Reaction Scheme 7, an intermediate thiazole tailpiece can be prepared by the condensation of compound (20) with bromo alkyl ester (21) in the presence of 1,4-dioxane followed by cyclization to give thiazole compound (22). The thiazole (22) then undergoes an ester reduction to give alcohol (13), which is further converted to thiazole sulfonyl ester (8) as described above in Reaction Scheme 3.

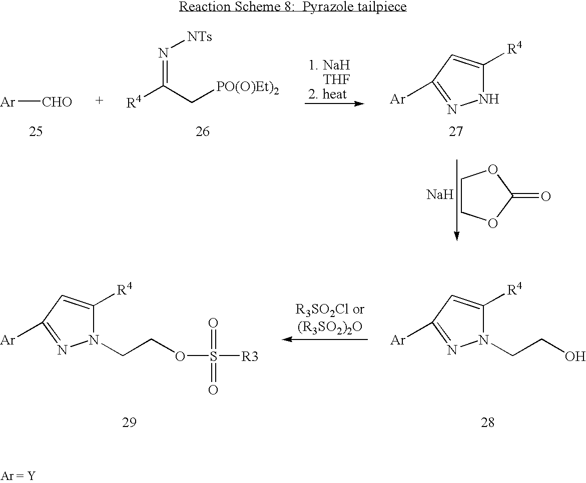

As shown in Reaction Scheme 8, an intermediate pyrazole tailpiece can be prepared by the condensation of arylaldehyde (25) with compound (26) in the presence of base followed by cyclization to give pyrazole compound (27). Compound (27) is treated with ethylene carbonate in the presence of base such as NaH to give alkylated compound (28), which is then converted to pyrazole sulfonyl ester (29) as described above in Reaction Scheme 3.

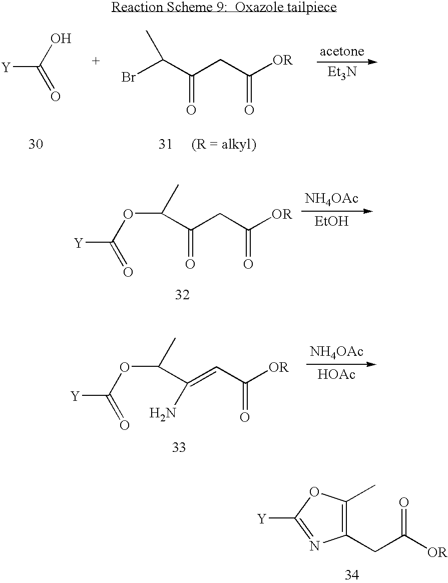

An alternative synthetic route to oxazole tailpiece is shown in Reaction Scheme 9. Carboxylic acid (30) is condensed with 2-bromo-3-oxopentanoate (preferably methyl ester) (31) to give ketoester (32). The latter is converted to an intermediate enamine (33) by treatment with anhydrous ammonium acetate. Subsequent cyclization of compound (33) in acetic acid in the presence of anhydrous ammonium acetate gives compound (34). The use of anhydrous ammonium acetate obtained by azeotropic evaporation with ethanol eliminates water in the reaction, which causes decarboxylation of compound (34). Additionally, some of the water liberated in the reaction is removed at the enamine stage. These modifications along with a simplified isolation procedure lead to higher yields of oxazole (34).

In the Schemes, Preparations and Examples below, various reagent symbols and abbreviations have the following meanings:
BINAP 2,2′-Bis(diphenylphosphino)-1,1′-binaphthyl
Boc t-butoxycarbonyl
CBZ benzyloxycarbonyl
DCM dichloromethane
DEAD diethyl azodicarboxylate
DI deionized
DIAD diisopropyl azodicarboxylate
DIPEA diisopropylethylamine
DMAP 4-dimethylamino pyridine
DMF N,N-dimethylformamide
DMSO dimethylsulfoxide
eq. (equiv) equivalent(s)
EDC 1-(3-dimethylaminopropyl)-3-ethylcarbodiimide HCl
ESI-MS electron spray ion-mass spectroscopy
Et ethyl
EtOAc ethyl acetate
FMOC 9-Fluororenylmethyl carbamate
HATU O-(7-azabenzotriazol-1-yl)-N,N,N′,N′-tetramethyluronium hexafluorophosphate
HOAc acetic acid
HOAT 1-hydroxy-7-azabenzotriazole
HOBT 1-hydroxybenzotriazole hydrate HPLC high performance liquid chromatography
HRMS high resolution mass
h hour(s)
LRMS low resolution mass
LAH lithium aluminum hydride
Me methyl
Ms methanesulfonyl
NBS N-bromosuccinimide
Pd₂(dba)₃ tris(dibenzylideneacetone) dipalladium(0)
Ph phenyl
Phe phenylalanine
Pr propyl
r.t (rt) room temperature
TBAF tetrabutylammonium fluoride
TBS tertbutyldimethylsilyl
TFA trifluoroacetic acid
TEA triethylamine
THF tetrahydrofuran
TLC thin-layer chromatography

### EXAMPLES

### Preparation of Intermediates

### TailPieces

### Preparation 1

### Toluene-4-sulfonic acid 2-(2-biphenyl-4-yl-5-methyl-oxazol-4-yl)ethyl ester

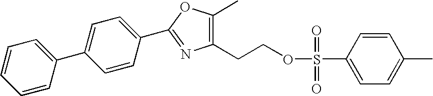

### Step A

### 4,5-Dimethyl-2-(4-bromophenyl)-oxazole oxide

A solution of 2,3-butanedione monooxime (50 g, 0.49 mol) and 4-bromo-benzaldehyde (101 g, 0.54 mol) in acetic acid (500 mL) is cooled to 0° C. and then gaseous HCl is bubbled through the solution for 35 min while the reaction is stirred in an ice bath. Diethyl ether (500 mL) is added to the reaction to precipitate the product, and the resultant slurry is stirred 45 min at 0° C. before being filtered. The solids are rinsed with Et₂O (50 mL), taken up in water (1 L), and conc. NH₄OH (60 mL) is added to the slurry. This mixture is extracted with CHCl₃. The organic layer is dried (MgSO₄) and concentrated to give 97.4 g (74%) of 4,5-dimethyl-2-(4-bromophenyl)-oxazole oxide as a white solid. This compound should be used directly within 24-48 h. ¹H NMR (500 MHz, CDCl₃) [symbol] 8.34 (d, J=9.0 Hz, 2H), 7.61 (d, J=9.0 Hz, 2H), 2.35 (s, 3H), 2.20 (s, 3H). HRMS (TOF) m/z calculated for C₁₁H₁₁⁷⁹BrNO₂: 267.997, found 267.9951.

### Step B

### 2-(4-Bromophenyl-4-(chloromethyl)-5-methyloxazole

A solution of 4,5-dimethyl-2-(4-bromophenyl)-oxazole oxide (96.6 g, 0.36 mol) in CHCl₃ (0.90 L) is treated dropwise with phosphorous oxychloride (61.1 g, 0.40 mol) allowing the reaction to exotherm and then is stirred at reflux for 30 min. The reaction is cooled to room temperature and washed with water (2×1 L). The combined aqueous ishes are back extracted with CH₂Cl₂ (2×400 mL). The organic layers are dried (MgSO₄) and concentrated to give crude product that is recrystallized from hot hexanes (300 mL), decanting the hot supernatant away from a dark oily material. The remaining dark oil is agitated in additional hot hexanes (200 mL), and the combined supernatants are cooled to 0° C. The product is isolated by filtration as a lime-green powder (74.2 g, 72%): Rf=0.39 in 20% ethyl acetate/hexanes. ¹H NMR (500 MHz, CDCl₃) δ 7.88-7.86 (m, 2H), 7.59-7.56 (m, 2H), 4.54 (s, 2H), 2.42 (s, 3H); HRMS (FAB) m/z calculated for C₁₁H₁₀⁷⁹BrClNO: 285.9634, found 285.9641.

### Step C

### 2-(4-Bromophenyl)-5-methyl-4-oxazoleacetic acid

To a solution of 2-(4-bromophenyl-4-(chloromethyl)-5-methyloxazole (64.8 g, 0.23 mol) in DMF (400 mL) is added powdered potassium cyanide (22.1 g, 0.34 mol) and potassium iodide (28.6 g, 0.17 mol), and the resultant mixture is heated to 85° C. for 3.5 h. The reaction mixture is cooled to room temperature. Potassium carbonate (5 g) is dissolved in water (800 mL) and added dropwise to the reaction to precipitate 2-(4-bromophenyl-4-(cyanomethyl)-5-methyloxazole (stir vigorously 15 min following addition) which is isolated by filtration and washed with water (2×400 mL). The crude 2-(4-bromophenyl-4-(cyanomethyl)-5-methyloxazole is used in the next step without purification. ¹H NMR (300 MHz, CDCl₃) 7.85 (m, 2H), 7.58 (m, 2H), 3.64 (s, 3H), 2.43 (s, 3H).

The crude 2-(4-bromophenyl-4-(cyanomethyl)-5-methyloxazole (assume 0.22 mol) is combined with 2-methoxyethanol (630 mL) and 85% solid KOH (74.6 g, 1.33 mol) in water (360 mL) is added to the reaction. The mixture is heated to reflux for 3 h, cooled, quenched with 2 M HCl (500 mL), and extracted with CH₂Cl₂. The organic layer is dried (MgSO₄) and concentrated, using toluene to remove residual 2-methoxyethanol azeotropically. The crude product (57.3 g) is recrystallized from toluene (450 mL) to give 39.8 g (60%) of 2-(4-bromophenyl)-5-methyl-4-oxazoleacetic acid as an off-white powder. Rf=0.23 in 10% MeOH/CH₂Cl₂; ¹H NMR (500 MHz, CDCl₃) 9.00 (br s, 1H), 7.85-7.83 (m, 2H), 7.58-7.56 (m, 2H), 3.62 (s, 3H), 2.36 (s, 3H).

### Step D

### 2-(4-Bromophenyl)-5-methyl-4-oxazoleethanol

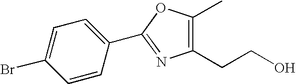

A solution of 2-(4-bromophenyl)-5-methyl-4-oxazoleacetic acid (39.1 g, 0.13 mol) in dry THF (175 mL) is treated dropwise with borane/THF complex (227 mL of a 1.0 M solution in THF, 1.3 mol) over 2 h at about 35° C. After stirring 2 h at room temperature under N₂, the reaction is quenched with slow addition of methanol (60 mL) and stirred overnight at room temperature. The reaction is diluted with 1 N NaOH (50 mL) and extracted with CH₂Cl₂ (2×200 mL). The organic layer is washed with H₂O (3×100 mL), dried (MgSO₄), and concentrated. The crude product (38.7 g) is recrystallized from toluene (200 mL, ish solid with cold hexanes) to give 26.9 g (72%) of 2-(4-bromophenyl)-5-methyl-4-oxazoleethanol as a white powder. Rf=0.37 in 10% MeOH/CH₂Cl₂. ¹H NMR (500 MHz, CDCl₃) [symbol] 84-7.82 (m, 2H), 7.57-7.55 (m, 2H), 3.91 (q, J=5.5 Hz, 2H), 3.14 (t, J=6 Hz, OH), 2.72 (t, J=5.5 Hz, 2H), 2.33 (s, 3H).

### Step E

### 2-(Biphenyl-4-yl-5-methyl-oxazol-4-yl)ethanol

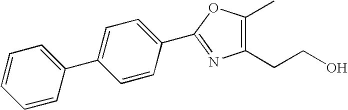

2-(4-Bromophenyl)-5-methyl-4-oxazoleethanol (10.0 g, 35.0 mmol) and phenylboronic acid (4.5 g, 38.0 mmol) are dissolved in n-propanol (120 mL) before adding triphenylphosphine (165.2 mg, 0.63 mmol), palladium acetate (46 mg, 2.1 mmol), and Na₂CO₃ (4.5 g, 42 mmol dissolved in 30 mL distilled H₂O). The solution is heated at reflux and stirred for 1.5 h. After cooling to ambient temperature, the mixture is concentrated and then partitioned between CH₂Cl₂ (100 mL) and 1N NaOH (100 mL). The aqueous phase is extracted with CH₂Cl₂ (2×50 mL). The combined organic phases are dried (MgSO₄) and concentrated under reduced pressure to provide 2-(4-biphenyl)-5-methyl-4-oxazoleethanol (9.5 g, 97% yield) as a white solid which is used directly without further purification. ¹H NMR (500 MHz, CDCl₃) [symbol] 8.01 (d, 2H), 7.77-7.50 (m, 4H), 7.46 (m, 2H), 7.38 (m, 1H), 3.91 (q, J=5.5 Hz, 2H), 3.18 (t, J=6 Hz, OH), 2.72 (t, J=5.5 Hz, 2H), 2.33 (s, 3H).

### Step F

### Toluene-4-sulfonic acid 2-(2-biphenyl-4-yl-5-methyl-oxazol-4-yl)ethyl ester

To a solution of 2-(biphenyl-4-yl-5-methyl-oxazol-4-yl)ethanol (15.8 g, 56.6 mmol) in CH₂Cl₂ (250 mL) at room temperature under N₂ is added pyridine (14.7 g, 185 mmol, 15.0 mL), DMAP (2.03 g, 16.6 mmol), and then tosyl anhydride (24.57 g, 75.2 mmol) portion wise. The reaction exothermed to 32° C. and is stirred 30 min before additional tosyl anhydride (2.3 g) is added. The mixture is diluted with CH₂Cl₂ (100 mL) and stirred vigorously with 1N HCl (150 mL) for 15 min. The organic phase is dried (MgSO₄) and filtered through a pad of silica gel (100 mL, packed with CH₂Cl₂). The silica gel is eluted with ethyl acetate (100 mL), and the solution is concentrated to give toluene-4-sulfonic acid 2-(2-biphenyl-4-yl-5-methyl-oxazol-4-yl)ethyl ester as a white solid (23.3 g, 95%). Rf=0.51 in 60% ethyl acetate/hexanes. ¹H NMR (400 MHz, CDCl₃) [symbol] 7.97 (d, 2H), 7.70 (d, 2H), 7.66 (t, 2H), 7.65 (d, 2H), 7.51 (t, 1H), 7.42 (d, 2H), 7.24 (d, 2H), 4.37 (t, 2H), 2.88 (t, 2H), 2.37 (s, 3H), 2.26 (s, 3H).

### Preparation 2

### Toluene-4-sulfonic acid 2-(4-Bromophenyl-5-methyl-oxazol-4-yl)ethyl ester

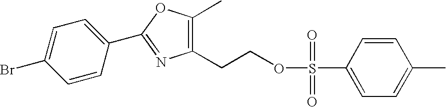

The title compound is prepared from 2-(4-bromophenyl)-5-methyl-4-oxazoleethanol according to Procedure 1, Step F: MS (ESI) m/z 436.0 (M+H)⁺.

The following intermediate compounds are prepared by a substantially similar manner as described in Preparations 1 and 2.

### 2-(3-Bromophenyl)-5-methyl-4-oxazoleethanol

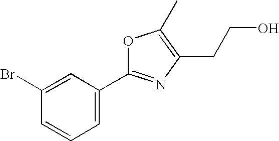

¹H NMR (300 MHz, d₆-DMSO) [symbol] 7.99 (s, 1H), 7.88 (d, J=7.7 Hz, 1H), 7.64 (d, J=7.7 Hz, 1H), 7.44 (t, J=7.7 Hz, 1H), 4.61 (t, J=5.5 Hz, OH), 3.63 (q, J=5.5 Hz, 2H), 2.60 (t, J=6.6 Hz, 2H), 2.32 (s, 3H);

### Toluene-4-sulfonic acid 2-(3-bromophenyl-5-methyl-oxazol-4-yl)ethyl ester

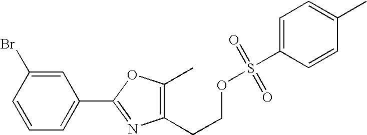

¹H NMR (400 MHz, CDCl₃) [symbol] 8.01 (t, J=1.6 Hz, 1H) 7.80 (dt, J=7.6 Hz, J=1.6 Hz, 1H), 7.66 (d, J=8.4 Hz, 2H), 7.53 (dt, J=7.6 Hz, J=1.6 Hz, 1H), 7.30 (t, J=7.6 Hz, 1H), 7.19 (d, J=8.4 Hz, 2H), 4.30 (t, J=6.4 Hz, 2H), 2.82 (t, J=6.4 Hz, 2H), 2.31 (s, 3H), 2.24 (s, 3H); MS (ESI) m/z 436.0 (M+H)⁺.

### Toluene-4-sulfonic acid 2-(2-biphenyl-3-yl-5-methyl-oxazol-4-yl)ethyl ester

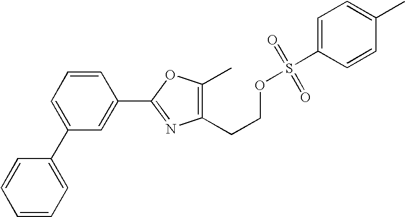

¹H NMR (300 MHz, CDCl₃) [symbol] 7.99 (s, 1H), 7.80 (d, J=8.0 Hz, 1H), 7.64 (d, 8.0 Hz, 1H), 7.51 (d, J=9.0 Hz, 1H), 7.30 (t, J=8.0 Hz, 1H), 7.20 (d, J=9.0 Hz, 1H), 4.30 (t, J=7.0 Hz, 2H), 2.80 (t, J=7.0 Hz, 2H), 2.30 (s, 3H), 2.23 (s, 3H).

### 2-(5-Methyl-2-thiophen-2-yl-4-oxazoleethanol

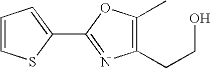

¹H NMR (500 MHz, CDCl₃): [symbol] 7.54 (m, 1H), 7.33 (m, 1H), 7.03 (m, 1H), 3.87 (t, J=5.8 Hz, 2H), 3.5 (s, 1H), 2.67 (t, J=5.8 Hz, 2H), 2.25 (s, 3H)

### Toluene-4-sulfonic acid 2-(5-methyl-2-thiophen-2-yloxazol-4-yl)ethyl ester

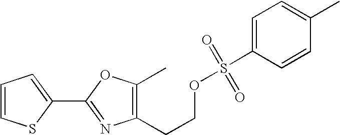

¹H NMR (400 MHz, CDCl₃): [symbol] 7.67 (d, J=8.3 Hz, 2H), 7.51 (dd, J=3.8, 1.4 Hz, 1H), 7.37 (dd, J=4.9, 1.2 Hz, 1H), 7.21 (d, J=7.9 Hz, 2H), 7.08 (dd, J=4.8, 3.5 Hz, 1H), 4.28 (t, J=6.3 Hz, 2H), 2.80 (t, J=6.3 Hz, 2H), 2.28 (s, 3H), 2.26 (s, 3H); mp 107-109° C.

### 2-[2-(4-Benzyloxy-phenyl)-5-methyl-oxazol-4-yl]-ethanol

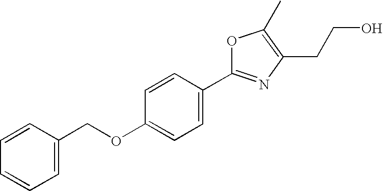

¹H NMR (500 MHz, CDCl₃) δ 7.91 (d, 2H, J=8.60 Hz), 7.45-34 (m, 5H), 7.02 (d, 2H, J=8.60 Hz), 5.11 (s, 2H), 3.91 (t, 2H, J=5.7 Hz), 2.71 (t, 2H, J=5.7 Hz), 2.31 (s, 3H); MS (ES⁺) Calculated for C₁₉H₂₀NO₃: Found m/e 310 (M+1, 100%).

### Toluene-4-sulfonic acid 2-[2-(4-benzyloxy-phenyl)-5-methyl-oxazol-4-yl]-ethyl ester

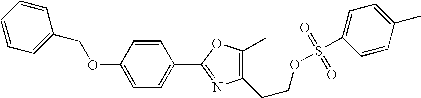

¹H NMR (500 MHz, CDCl₃) [symbol] 7.80-7.78 (m, 2H), 7.67-7.65 (m, 2H), 7.45-7.34 (m, 5H), 7.25-7.17 (m, 2H), 7.02-6.99 (m, 2H), 5.12 (s, 2H), 4.29 (t, 2H, J=6.45 Hz), 2.80 (t, 2H, J=6.45 Hz), 2.27 (s, 3H), 2.22 (s, 3H); HRMS (ES⁺) m/z exact mass calculated for C₂₆H₂₆NO₅S 464.1532, found 464.1531; Anal. Calculated for C₂₆H₂₅NO₅S: C, 67.37; H, 5.44; N, 3.02. Found C, 66.59; H, 5.33; N, 3.06.

### Preparation 3

### Toluene-4-sulfonic acid 2-(5-methyl-2-phenethyl-oxazol-4-yl)-ethyl ester

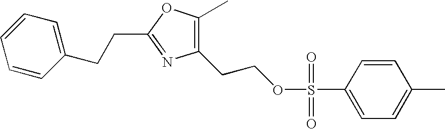

### Step A

### 2-(3-Phenyl-propionylamino)-succinic acid 4-methyl ester

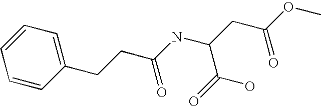

Methyl L-aspartate (15.0 g, 0.082 mol), DI water (245 mL), acetone (20 mL), and Na₂CO₃ (30.8 g, 0.286 mol) are combined and cooled the solution to 5° C. The compound 3-phenyl-propionyl chloride (13.3 mL, 0.089 mol) is added dropwise via addition funnel over 10 min. The reaction is allowed to warm to ambient temperature and stir for 2 h. Conc. HCl (50 mL) is added to the thick slurry until the pH is ≦4.0. The reaction mixture is extracted with CH₂Cl₂ (3×100 mL). The combined organic layers are washed with water, dried (MgSO₄), filtered, and concentrated. The clear, colorless oil is used without further purification. ¹H NMR (400 MHz, CDCl₃) [symbol] 7.92 (br s, 1H), 7.28-7.17 (m, 5H), 6.57 (d, J=7.6 Hz, 1H), 4.87 (m, 1H), 3.67 (s, 3H), 2.96 (t, J=7.6 Hz, 2H), 2.89 (A of ABX, J_{AB}=17.6 Hz, J_{AX}=4.8 Hz, 1H), 2.88 (B of ABX, J_{BA}=17.6 Hz, J_{BX}=4.0 Hz, 1H), 2.69 (t, J=7.6 Hz, 2H); MS (EI+) 280 (M+H), 302 (M+H+Na).

### Step B

### 4-Oxo-3-(3-phenyl-propionylamino)-pentanoic acid methyl ester

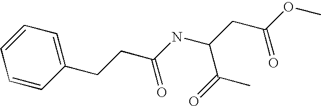

2-(3-Phenyl-propionylamino)-succinic acid 4-methyl ester (10 g, 36 mmol), pyridine (50 mL) and acetic anhydride (45 mL) are combined in a 500 mL flask. The reaction mixture is heated at 90° C. for 2 h and then cooled to ambient temperature. After concentrating the reaction mixture under reduced pressure, DI water is added (100 mL). The reaction mixture is partitioned between water and CH₂Cl₂ (200 mL). The organic phase is washed with 1N HCl (50 mL), dried (MgSO₄), filtered, and concentrated. The material is used without further purification. ¹H NMR (400 MHz, CDCl₃) [symbol] 7.31-7.20 (m, 5H), 6.79 (br d, J=7.6 Hz, 1H), 4.72 (X of ABX, 1H), 3.65 (s, 3H), 3.01-2.93 (m, 3H), 2.71-2.62 (m, 3H), 2.11 (s, 3H); MS (EI) 278.1 (M+H).

### Step C

### (5-Methyl-2-phenethyl-oxazol-4-yl)-acetic acid methyl ester

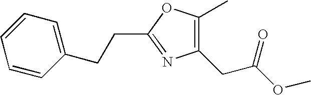

In a 100 mL flask, 4-oxo-3-(3-phenyl-propionylamino)-pentanoic acid methyl ester (10 g, 36 mmol) and acetic anhydride (28 mL) are combined. Following addition of concentrated H₂SO₄ (1 mL), the solution is heated to 90° C. for 30 min and cooled to ambient temperature. The reaction is slowly diluted with DI water (30 mL, potential exotherm). The reaction mixture is partitioned between CH₂Cl₂ (150 mL) and water (150 mL). The organic phase is washed with DI water, 10% NaHCO₃ (aq), brine (150 mL), and then is dried (MgSO₄) and concentrated to a brown oil. The residue is purified by column chromatography (600 mL SiO₂, 35% EtOAc/hexanes) to provide the desired product (3.25 g) as a pale yellow oil. ¹H NMR (400 MHz, CDCl₃) [symbol] 7.33-7.20 (m, 5H), 3.72 (s, 3H), 3.47 (s, 2H), 3.08-2.96 (m, 4H), 2.24 (s, 3H); MS (EI+) 260 (M+H).

### Step D

### (5-Methyl-2-phenethyl-oxazol-4-yl)-acetic acid

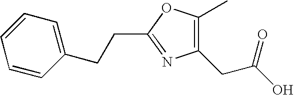

(5-Methyl-2-phenethyl-oxazol-4-yl)-acetic acid methyl ester (8.75 g, 33.8 mmol), in MeOH (120 mL) is treated with 5N NaOH (40 mL), and then the solution is warmed to 40° C. After 40 min, the reaction mixture is concentrated. The residue is suspended in water (75 ml) and acidified to pH=1 with 5N HCl. The mixture is extracted with EtOAc (2×), dried (MgSO₄), and concentrated to provide 5.25 g (63%) of the product as an off-white solid. ¹H NMR (400 MHz, CDCl₃) ¹H NMR (400 MHz, CDCl₃) [symbol] 7.33-7.20 (m, 5H), 3.52 (s, 2H), 3.06-3.03 (m, 4H), 2.24 (s, 3H).

### Step E

### 2-(5-Methyl-2-phenethyl-oxazol-4-yl)-ethanol

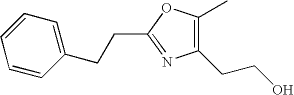

BH₃-THF complex (49 mL of a 1.0 M solution in THF) is added dropwise via addition funnel over 50 min to a solution of (5-methyl-2-phenethyl-oxazol-4-yl)-acetic acid (5.05 g, 20.6 mmol) in THF (35 mL). The reaction mixture is stirred at ambient temperature for 3 h, and then quenched with MeOH (12 mL). After heating at 50° C. for 2 h, the reaction mixture is cooled to ambient temperature, and then partitioned between CH₂Cl₂ and 1N NaOH. The organic phase is washed with brine (1×50 mL), dried over MgSO₄ and concentrated to obtain a residue, which is purified by column chromatography (500 mL SiO₂, 35% EtOAc/hexanes) to provide 3.99 g (84%) of the desired product as a clear, colorless oil. ¹H NMR (400 MHz, CDCl₃) [symbol] 7.33-7.20 (m, 5H), 3.84 (q, J=5.6 Hz, 2H), 3.06-2.67 (m, 4H), 2.62 (t, J=5.6 Hz, 2H), 2.22 (s, 3H); MS (EI+) 232.19 (M+H); 254.15 (M+H+Na).

### Step F

### Toluene-4-sulfonic acid 2-(5-methyl-2-phenethyl-oxazol-4-yl)-ethyl ester

A solution of 2-(5-methyl-2-phenethyl-oxazol-4-yl)-ethanol (1.2 g, 5.19 mmol) in CH₂Cl₂ at 0° C. is treated with pyridine (1.64 g, 20.7 mmol, 1.68 mL), DMAP (190 mg, 1.56 mmol), and tosyl anhydride (2.2 g, 6.75 mmol). The reaction is warmed to ambient temperature and, after 90 min, the solution is filtered through a pad of silica gel (rinsed with CH₂Cl₂). The product is used without further purification. ¹H NMR (400 MHz, CDCl₃) [symbol] 7.73 (d, J=8.4 Hz, 2H), 7.31-7.17 (m, 7H), 4.21 (t, J=6.8 Hz, 2H), 3.01-2.88 (m, 4H), 2.75 (t, J=6.8 Hz, 2H), 2.43 (s, 3H), 2.19 (s, 3H).

The following intermediate compounds are prepared by a substantially similar manner as described in Preparations 3.

### 2-(2-Cyclohexyl-5-methyl-oxazol-4-yl)-ethanol

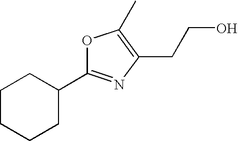

¹H NMR (400 MHz, CDCl₃) δ 3.73 (t, J=6.8 Hz, 2H), 2.58 (tt, J=11.6, 3.6 Hz, 1H), 2.54 (t, J=6.8 Hz, 2H), 2.13 (s, 3H), 1.93-1.89 (m, 2H), 1.74 (dt, J=12.8, 3.6 Hz, 2H), 1.67-1.62 (m, 1H), 1.41 (qd, J=12.0, 3.2 Hz, 1H), 1.33-1.17 (m, 4H); MS (EI+) 210.1 (M+H).

### Toluene-4-sulfonic acid 2-(2-cyclohexyl-5-methyl-oxazol-4-yl)-ethyl ester

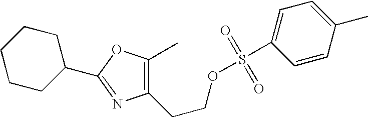

¹H NMR (400 MHz, CDCl₃) δ 7.67 (d, J=8.4 Hz, 2H), 7.27 (d, J=8.4 Hz, 2H), 4.16 (t, J=6.8 Hz, 2H), 2.70 (t, J=6.8 Hz, 2H), 2.56 (tt, J=11.6, 3.6 Hz, 1H), 2.39 (s, 3H), 2.13 (s, 3H), 1.93-1.89 (m, 2H), 1.74 (dt, J=12.8, 3.6 Hz, 2H), 1.67-1.62 (m, 1H), 1.41 (qd, J=12.0, 3.2 Hz, 1H), 1.33-1.17 (m, 4H); MS (EI+) 364.1 (M+H)⁺.

### 2-[5-Methyl-2-(1-methylcyclohexyl)oxazol-4-yl]ethanol

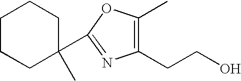

MS (EI+) 224.1 (M+H)⁺.

### Toluene-4-sulfonic acid 2-[5-methyl-2-(1-methylcyclohexyl)oxazol-4-yl]ethyl ester

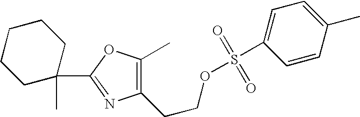

¹H NMR (400 MHz, CDCl₃) [symbol] 7.73 (d, J=8.4 Hz, 2H), 7.30 (t, J=8.4 Hz, 2H), 4.12 (t, J=6.6 Hz, 2H), 2.76 (t, J=6.6 Hz, 2H), 2.42 (s, 3H), 2.17 (s, 3H), 2.61-2.02 (m, 2H), 1.56-1.30 (m, 8H), 1.19 (s, 3H); MS (EI) 378.2 (M+H)⁺.

### 2-[5-Methyl-2-(tetrahydro-pyran-4-yl)-oxazol-4-yl]-ethanol

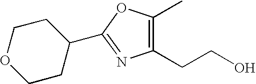

MS (EI) 212.2 (M+H)⁺.

### Toluene-4-sulfonic acid 2-[5-methyl-2-(tetrahydro-pyran-4-yl)-oxazol-4-yl]-ethyl ester

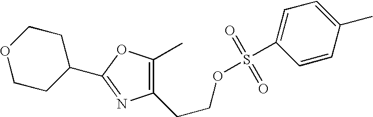

MS (EI) 366.2 (M+H)⁺.

### 2-(2-Benzyl-5-methyl-oxazol-4-yl)-ethanol

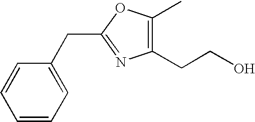

MS (EI) 218.0 (M+H)⁺.

### Toluene-4-sulfonic acid 2-(2-benzyl-5-methyl-oxazol-4-yl)-ethyl ester

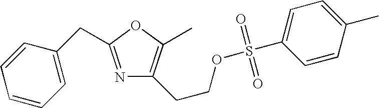

MS (EI) 372.1 (M+H)⁺.

### 2-(2-Benzo[b]thiophen-2-yl-5-methyl-oxazol-4-yl)-ethanol

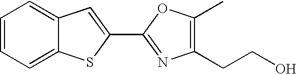

¹H NMR (CDCl₃) [symbol] 7.81 (m, 3H), 7.38 (m, 2H), 3.94 (m, 2H), 3.07 (br s, 1H), 2.73 (t, 2H, J=6 Hz), 2.34 (s, 3H); ¹³C NMR (CDCl₃) [symbol] 155.9, 145.0, 140.5, 139.8, 134.5, 129.9, 125.9, 125.1, 124.7, 123.7, 122.7, 61.9, 28.5, 10.4; MS (EI) 260.1 (M+H)⁺.

### Toluene-4-sulfonic acid 2-(2-benzo[b]thiophen-2-yl-5-methyl-oxazol-4-yl)-ethyl ester

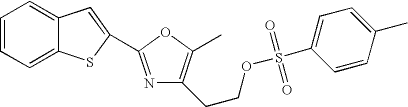

¹H NMR (CDCl₃) [symbol] 7.85 (m, 1H), 7.84 (m, 1H), 7.75 (s, 1H), 7.67 (d, 2H, J=8 Hz), 7.39 (m, 2H), 7.21 (m, 2H), 4.31 (t, 2H, J=2 Hz), 2.83 (t, 2H, J=6 Hz), 2.32 (s, 3H), 2.19 (s, 3H).

### 2-(5-Methyl-2-naphthalen-2-yl-oxazol-4-yl)-ethanol

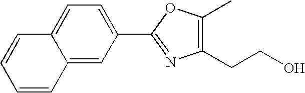

HRMS Calcd for C₁₆H₁₆NO₂: m/z 254.1181. Found: 254.1167.

### 2-[5-Methyl-2-(4-trifluoromethyl-phenyl)-oxazol-4-yl]-ethanol

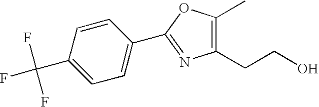

MS (EI) 272 (M+H)⁺.

### Toluene-4-sulfonic acid 2-[5-methyl-2-(4-trifluoromethyl-phenyl)-oxazol-4-yl]-ethyl ester

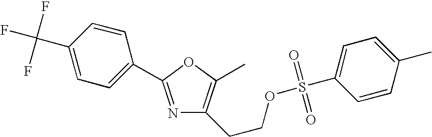

MS (EI) 426 (M+H)⁺.

### 2-[2-(4-Butoxy-phenyl)-5-methyl-oxazol-4-yl]-ethanol

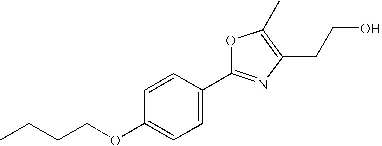

MS (EI) 276 (M+H)⁺.

### Toluene-4-sulfonic acid 2-[2-(4-butoxy-phenyl)-5-methyl-oxazol-4-yl]-ethyl ester

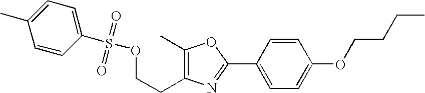

MS (EI) 430 (M+H)⁺.

### 2-(2-Bromophenyl-5-methyl-oxazol-4-yl)-ethanol

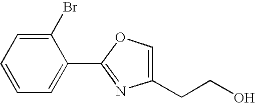

MS (EI) 282.1 (M+H)⁺.

### Toluene-4-sulfonic acid 2-(2-bromophenyl-5-methyl-oxazol-4-yl)ethyl ester

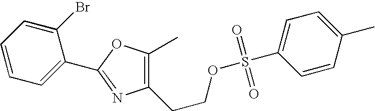

MS (EI) 438.1 (M+H)⁺.

### Preparation 4

### Toluene-4-sulfonic acid 2-[2-(6-chloro-pyridin-3-yl)-5-methyl-oxaxol-4-yl]-ethyl ester

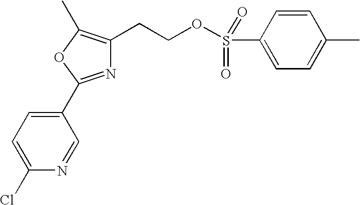

### Step A

### 3-[2-(6-Chloro-pyridin-3-yl)-5-methyl-oxazole-4-yl]-acetic acid methyl ester

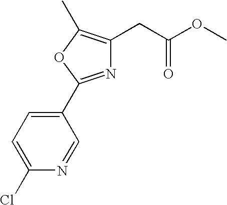

According to Preparation 3, Steps A to C, 6-chloronicotinic acid is converted into the title compound. MS (ESI) m/z 267 (M+H)⁺.

### Step B

### 3-[2-(6-Chloro-pyridin-3-yl)-5-methyl-oxazole-4-yl]-ethanol

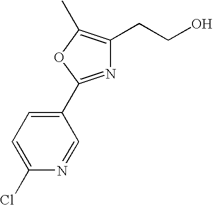

A solution of 3-[2-(6-chloro-pyridin-3-yl)-5-methyl-oxazole-4-yl]-acetic acid methyl ester (500 mg, 1.88 mmol) in THF (20 mL) at 0° C. is treated LAH (90 mg, 2.3 mmol). The reaction mixture is stirred for 1 h and is quenched with water (0.1 mL), 15% NaOH (0.1 mL), and water (0.3 mL). The mixture is filtered through Celite and concentrated to give the title alcohol which as used in the next step without further purification. MS (ESI) m/z 239 (M+H)⁺.

### Step C

### Toluene-4-sulfonic acid 2-[2-(6-chloro-pyridin-3-yl)-5-methyl-oxaxol-4-yl]-ethyl ester

A solution of crude 3-[2-(6-chloro-pyridin-3-yl)-5-methyl-oxazole-4-yl]-ethanol (1.88 mmol max) in CH₂Cl₂ (10 mL) is treated with para-toluenesulfonyl chloride (0.4 g, 2.3 mmol), DMAP (40 mg), and triethylamine (0.4 mL, 2.82 mmol). The reaction mixture is stirred at ambient temperature overnight and is diluted with CH₂Cl₂ (20 mL). The mixture is washed with water, and the organic layer is dried (MgSO₄), filtered, and concentrated. The crude product is purified by silica gel chromatography (hexanes/EtOAc 10/1 to 2/1) to afford the title compound (295 mg, 40% over two steps). MS (ESI) m/z 393 (M+H)⁺.

### Preparation 5

### Toluene-4-sulfonic acid 2-{5-methyl-2-[4-(methyl-phenyl-amino)-phenyl]-oxazol-4-yl}-ethyl ester

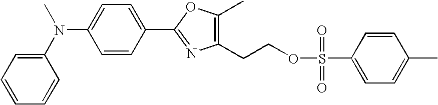

### Step A

### 4-(2-Benzyloxy-ethyl)-2-(4-bromo-phenyl)-5-methyl-oxazole

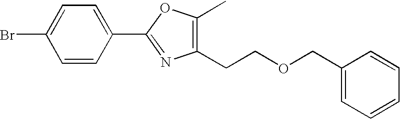

A solution of 2-[2-(4-bromo-phenyl)-5-methyl-oxazol-4-yl]-ethanol (3.17 g, 11.2 mmol) in DMF (25 mL) is treated with NaH (0.67 g, 60% oil dispersion) at 0° C. and stirred for 5 min. Benzyl bromide (2.90 g, 16.9 mmol) is added, and the resulting mixture is stirred at room temperature for 3 h. The reaction is quenched with water, and the mixture is extracted with EtOAc (2×150 mL). The combined organics are dried (Na₂SO₄), concentrated, and purified by silica gel chromatography column (10% EtOAc/hexanes) to yield the title compound as an oil (2.50 g, 60%).

### Step B

### {4-[4-(2-Benzyloxy-ethyl)-5-methyl-oxazol-2-yl]-phenyl}-methyl-phenyl-amine

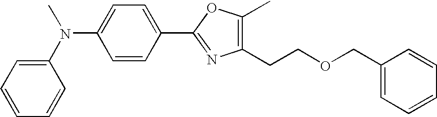

A solution of 4-(2-benzyloxy-ethyl)-2-(4-bromo-phenyl)-5-methyl-oxazole (200 mg, 0.538 mmol) in toluene (5.0 mL) in a seal tube under nitrogen gas flow is treated with Pd(OAc)₂ (50 mg), 2-(di-t-butylphosphino)biphenyl (20 mg), N-methyl aniline (115 mg, 1.08 mmol), and sodium t-butoxide (104 mg, 1.08 mmol). The tube is sealed and heated at 105° C. for 14 h. The mixture is cooled and purified directly by silica gel column chromatography (30-50% EtOAc/hexanes) to yield the title compound (195 mg, 91%). MS (ESI) m/z 399.3 (M+H)⁺.

### Step C

### 2-{5-Methyl-2-[4-(methyl-phenyl-amino)-phenyl]-oxazol-4-yl}-ethanol

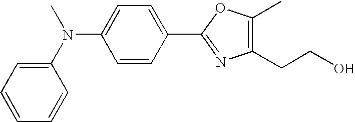

A solution of {4-[4-(2-benzyloxy-ethyl)-5-methyl-oxazol-2-yl]-phenyl}-methyl-phenyl-amine (195 mg, 0.490 mmol) in THF (2 mL) and EtOH (10 mL) is treated a slurry of Pd/C (200 mg) in EtOH (2 mL). The resulting mixture is treated with hydrogen under balloon pressure for 14 h and filtered through a pad of Celite. The filtrate is concentrated, and crude product is purified by silica gel chromatography column (50% EtOAc/hexanes) to yield the title compound (91 mg, 60%).

### Step D

### Toluene-4-sulfonic acid 2-{5-methyl-2-[4-(methyl-phenyl-amino)-phenyl]-oxazol-4-yl}-ethyl ester

A solution of 2-{5-methyl-2-[4-(methyl-phenyl-amino)-phenyl]-oxazol-4-yl}-ethanol (91 mg, 0.30 mmol) in CH₂Cl₂ (4.0 mL) is treated with para-toluenesulfonyl chloride (68 mg, 0.36 mmol), triethyl amine (0.20 mL) and a few crystals of DMAP. The resulting mixture is stirred at room temperature for 14 h and is quenched with water (0.2 mL). The mixture is purified directly by silica gel column chromatography (40% EtOAc/hexanes) to yield the title compound (120 mg, 83%). MS (ESI) m/z 463.1 (M+H)⁺.

The following intermediate compounds are prepared by a substantially similar manner as described in Preparation 5.

### Toluene-4-sulfonic acid 2-[5-methyl-2-(4-phenylamino-phenyl)-oxazol-4-yl]-ethyl ester

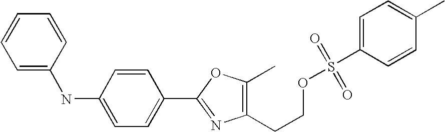

MS (ESI) m/z 449.1 (M+H)⁺.

### Toluene-4-sulfonic acid 2-[5-methyl-2-(4-morpholin-4-yl-phenyl)-oxazol-4-yl]-ethyl ester

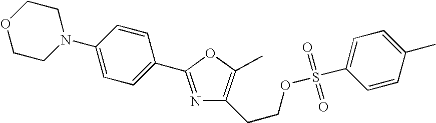

MS (ESI) m/z 443.1 (M+H)⁺.

### Preparation 6

### Toluene-4-sulfonic acid 2-[5-methyl-2-(4-phenoxy-phenyl)-oxazol-4-yl]-ethyl ester

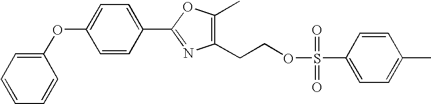

### Step A

### 4-(2-Benzyloxy-ethyl)-5-methyl-2-(4-phenoxy-phenyl)-oxazole

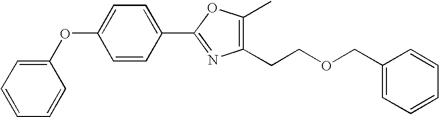

A mixture of 4-(2-benzyloxy-ethyl)-2-(4-bromo-phenyl)-5-methyl-oxazole (0.025 mol, 9.2 g), phenol (0.03 mol, 2.8 g), K₃PO₄ (0.05 mol, 10.6 g), 2-(di-tert-butylphosphino)biphenyl (1.8 mmol, 0.54 g) and Pd(OAc)₂ (1.2 mmol, 0.28 g) in toluene (350 mL) is degassed with nitrogen and heated at 100° C. for 18 h. Additional Pd(OAc)₂ (0.5 g) and phosphine ligand (1.0 g) are added, and the mixture is heated 5 h at 100° C. The reaction is concentrated and purified directly by silica gel chromatography (4/1 hexanes/ethyl acetate) to give the title compound (7.6 g).

### Step B

### Toluene-4-sulfonic acid 2-[5-methyl-2-(4-phenoxy-phenyl)-oxazol-4-yl]-ethyl ester

According to Preparation 5, Steps C to D, 4-(2-benzyloxy-ethyl)-5-methyl-2-(4-phenoxy-phenyl)-oxazole is converted into the title compound. ¹HNMR (400 MHz, CDCl₃) [symbol] 7.81 (d, 2H, J=9.1 Hz), 7.67 (d, 2H, J=8.2 Hz), 7.37 (t, 2H, J=8.2 Hz), 7.15 (m, 3H), 7.12 (m, 4H), 4.39 (t, 2H, J=6.4 Hz), 2.81 (t, 2H, 6.4 Hz), 2.30 (s, 3H), 2.25 (s, 3H).

### Preparation 7

### 4-Methyl-3-nitro-benzenesulfonic acid 2-[5-methyl-2-(4-nitro-phenyl)-oxazol-4-yl]-ethyl ester

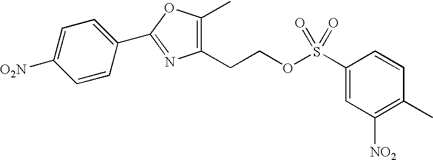

A mixture of potassium nitrate (3.0 g, 30 mmol, 2.7 equiv) and sulfuric acid (10 mL, 18 g, 94 mmol, 17 equiv) is cooled to 0° C. Toluene-4-sulfonic acid 2-(5-methyl-2-phenyl-oxazol-4-yl)-ethyl ester (4.00 g, 11.2 mmol, 1 equiv) is added and the ice bath is removed. The reaction mixture is heated with a heat gun until the tosylate dissolved. After 30 min, the solution is poured into H₂O (100 mL) and extracted with EtOAc (100 mL). The organic layer is dried (Na₂SO₄) and concentrated (75° C.) to an orange oil (4.41 g). The crude product is purified by silica gel flash chromatography (30-50% EtOAc/hexanes) to give the title compound as a yellow solid (3.64 g, 73%). MS (ESI) m/z 447 (M+H)⁺.

### Preparation 8

### Toluene-4-sulfonic acid 2-(5-methoxy-2-phenyl-oxazol-4-yl)-ethyl ester

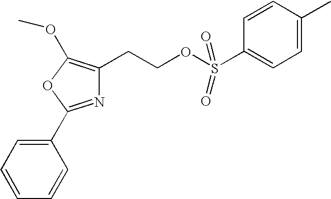

### Step A

### 2-Benzoylamino-succinic acid dimethyl ester

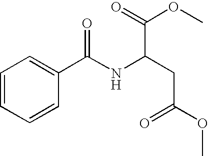

A mixture of benzoyl chloride (3.20 mL, 27.7 mmol), L-aspartic acid dimethyl ester (5.0 g, 25.2 mmol) and triethyl amine (5.3 mL, 38 mmol) in CH₂Cl₂ (50 mL) is stirred at ambient temperature for 2 h and diluted with water. The organic layer is dried (MgSO₄), filtered, and concentrated. The residue is purified by silica gel chromatography (hexanes/EtOAc 1/1) to afford a white solid (5.3 g, 79%). MS (ESI) m/z 266 (M+H)⁺.

### Step B

### 3-(5-Methoxy-2-phenyl-oxazol-4-yl)-acetic acid methyl ester

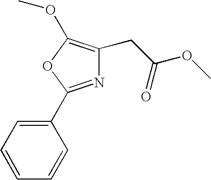

A mixture of 2-benzoylamino-succinic acid dimethyl ester (5.3 g, 20 mmol) in 1,2-dichloroethane (15 mL) is treated with P₂O₅ (5.3 g, 30 mmol) and Celite (3.2 g) and is heated at 85° C. for 2 h. The solvent is decanted and concentrated. The residue is dissolved in CH₂Cl₂ and washed with saturated aqueous NaHCO₃. The organic layer is dried (MgSO₄), filtered, and concentrated. The residue is purified by silica gel chromatography (hexanes/EtOAc 10/1 to 3/1) to afford the title compound (2.9 g, 59%). MS (ESI) m/z 247 (M+H)⁺.

### Step C

### 3-(5-Methoxy-2-phenyl-oxazol-4-yl)-ethanol

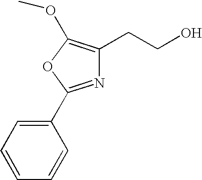

A suspension of LAH (0.56 g, 14.1 mmol) in THF (100 mL) at −78° C. is treated dropwise with a solution of 3-(5-methoxy-2-phenyl-oxazol-4-yl)-acetic acid methyl ester (2.9 g, 11.7 mmol) in THF (100 mL). After the addition is completed, the reaction mixture is warmed up to ambient temperature, cooled to −20° C., and quenched with H₂O (0.8 mL), 15% NaOH (0.8 mL), and H₂O (2.4 mL). The mixture is filtered through Celite and concentrated to the title compound as an oil. MS (ESI) m/z 220.3 (M+H)⁺.

### Step D

### Toluene-4-sulfonic acid 2-(5-methoxy-2-phenyl-oxazol-4-yl)-ethyl ester

A solution of crude 3-(5-methoxy-2-phenyl-oxazol-4-yl)-ethanol (11.7 mmol max) in CH₂Cl₂ (100 mL) is treated with para-toluenesulfonyl chloride (2.7 g, 14.0 mmol), DMAP (100 mg), and triethylamine (2.5 mL, 17.6 mmol). The reaction mixture is stirred at ambient temperature for 16 h and is washed with water. The organic layer is dried (MgSO₄), filtered, and concentrated. The residue is purified by silica gel chromatography (hexanes/EtOAc, 10/1 to 1/1) to afford the title compound (2.0 g, 46% over two steps). MS (ESI) m/z 374 (M+H)⁺.

The following intermediate compounds are prepared by a substantially similar manner as described in Preparation 8.

### 2-(2-Biphenyl-4-yl-5-methoxy-oxazol-4-yl)-ethanol

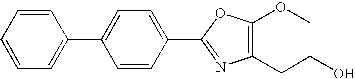

MS (ESI) m/z 296.0 (M+H)⁺.

### Toluene-4-sulfonic acid 2-(2-biphenyl-4-yl-5-methoxy-oxazol-4-yl)-ethyl ester

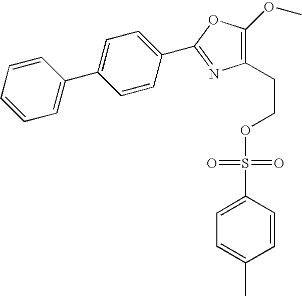

MS (ESI) m/z 450.1 (M+H)⁺.

### Preparation 9

### Toluene-4-sulponic acid 2-[5-methyl-2-(6-phenyl-pyridin-3-yl)thiazol-4-yl]ethyl ester

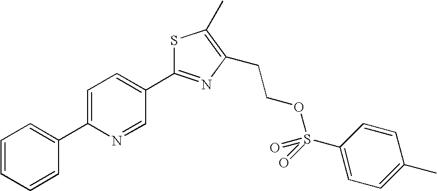

### Step A

### 2-Phenyl-5-cyanopyridine

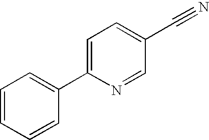

5-Cyano-2-chloropyridine (5.0 g, 36.1 mmol), phenylboronic acid (6.6 g, 54 mmol), tetrakis(triphenylphosphine) palladium (0) (0.5 g), and aqueous Na₂CO₃ (7.6 g), in toluene (100 mL) are heated at 90° C. for 16 h. The mixture is diluted with EtOAc and washed with H₂O. The organic layer is dried (MgSO₄), filtered, and concentrated. The residue is purified by silica gel chromatography (hexanes/EtOAc 2/1) to afford the title compound (6.1 g, 94%). MS (ESI) m/z 181 (M+H)⁺.

### Step B

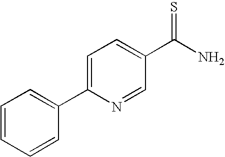

A mixture of 2-phenyl-5-cyanopyridine (6.0 g, 33 mmol) and thioacetamide (4.0 g, 53 mmol) in 4N HCl in 1,4-dioxane (50 mL) is heated at 98° C. for 20h. The reaction mixture is cooled and poured into aqueous saturated NaHCO₃. The precipitate is collected, washed with water, and dried under vacuum (60° C.) to afford the title compound as a yellow solid (7.0 g, 99%).

### Step C

### [5-Methyl-2-(6-phenyl-pyridin-3-yl)-thiazol-4-yl]-acetic acid methyl ester

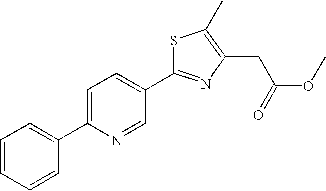

A mixture of 6-phenyl-thionicotinamide (7.0 g) and 4-bromo-3-oxo-pentanoic acid methyl ester (9.15 g, 35 mmol) in 1,4-dioxane (30 mL) is heated at reflux for 4 h. The reaction mixture is cooled, poured into aqueous saturated NaHCO₃, and extracted with CH₂Cl₂. The organic layer is dried (MgSO₄), filtered, and concentrated. The residue is purified by silica gel chromatography (hexanes/EtOAc, 2/1) to afford the title compound (6.0 g, 56%). MS (ESI) m/z 325 (M+H)⁺.

### Step D

### [5-Methyl-2-(6-phenyl-pyridin-3-yl)-thiazol-4-yl]-ethanol

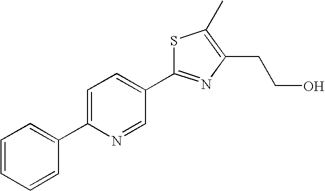

A solution of [5-methyl-2-(6-phenyl-pyridin-3-yl)-thiazol-4-yl]-acetic acid methyl ester (6.0 g, 18.5 mmol) in THF (500 mL) is added dropwise to a suspension of LAH (0.90 g, 22.2 mmol) in THF (300 mL) at −78° C. After the addition is completed, the reaction mixture is allowed to warm to ambient temperature, cooled to −20° C., and quenched sequentially with H₂O (1.1 mL), 15% NaOH (1.1 mL), and H₂O (3.3 mL). The mixture is filtered through Celite, and the filtrated is concentrated to give the title compound as an oil that is used directly in the next step.

### Step E

### Toluene-4-sulponic acid 2-[5-methyl-2-(6-phenyl-pyridin-3-yl)thiazol-4-yl]ethyl ester

A mixture of [5-methyl-2-(6-phenyl-pyridin-3-yl)-thiazol-4-yl]-ethanol (18.5 mmol max), para-toluenesulfonyl chloride (3.89 g, 20.5 mmol), DMAP (500 mg), and triethylamine (4.0 mL, 28.0 mmol) in CH₂Cl₂ (300 mL) is stirred at ambient temperature for 2.5 h. The reaction mixture is diluted with water, and the organic layer is separated, dried (MgSO₄), filtered, and concentrated. The residue is purified by silica gel chromatography (hexanes/EtOAc, 10/1 to 1/1) to afford the title compound as a solid (2.0 g, 46% over two steps). MS (ESI) m/z 451 (M+H)⁺.

The following intermediate compounds are prepared by a substantially similar manner as described in Preparation 9.

### 2-[5-Methyl-2-(5-phenyl-pyridin-3-yl)-thiazol-4-yl]-ethanol

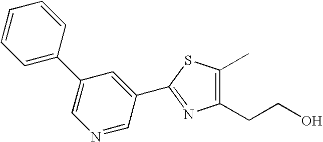

MS (ESI) m/z 297 (M+H)⁺.

### Toluene-4-sulfonic acid 2-[5-methyl-2-(5-phenyl-pyridin-3-yl)thiazol-4-yl]ethyl ester

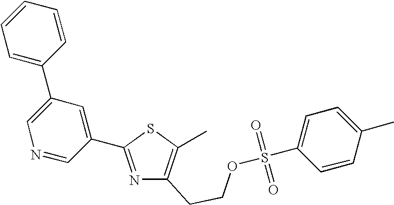

MS (ESI) m/z 451 (M+H)⁺.

### Toluene-4-sulfonic acid 2-[5-methyl-2-(6-phenoxy-pyridin-3-yl)-thiazol-4-yl]-ethyl ester

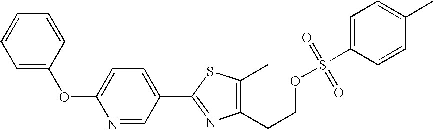

¹HNMR 400 MHz (CDCl₃) [symbol] 8.52 (2, 1H), 8.03 (d, 1H, J=6.9 Hz), 7.63 (d, 2H, J=8.6 Hz), 7.42 (t, 2H, J=7.7 Hz), 7.2 (m, 5H), 6.91 (1H, d, J=7.7 Hz), 4.37 (t, 2H, J=6.3 Hz), 3.02 (t, 2H, J=6.3 Hz), 2.39 (s, 3H), 2.28 (s, 3H).

### Toluene-4-sulfonic acid 2-[5-methyl-2-(6-morpholin-4-yl-pyridin-3-yl)-thiazol-4-yl]-ethyl ester

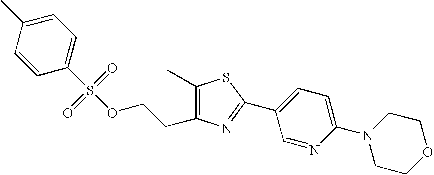

¹HNMR 400 MHz (CDCl₃) [symbol] 8.51 (s, 1H), 7.82 (d, 1H, J=8.5 Hz), 7.63 (d, 2H, J=7.8 Hz), 7.19 (d, 2h, J=7.8 Hz), 6.61 (d, 1H, J=8.5 Hz), 4.37 (t, 2H, J=6.5 Hz), 3.82 (t, 2H, J=5.2 Hz), 3.58 (t, 2H, J=5.2 Hz), 2.99 (t, 2H, J=6.5 Hz), 2.35 (s, 3H), 2.27 (s, 3H).

### 4-{5-Methyl-4-[2-(toluene-4-sulfonyloxy)-ethyl]-thiazol-2-yl}-piperazine-1-carboxylic acid tert-butyl ester

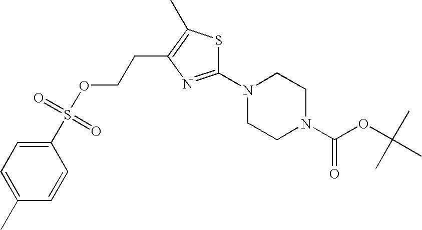

MS (ESI) m/z 482 (M+H)⁺.

### Toluene-4-sulfonic acid 2-[5-methyl-2-(4-methyl-piperazin-1-yl)-thiazol-4-yl]-ethyl ester

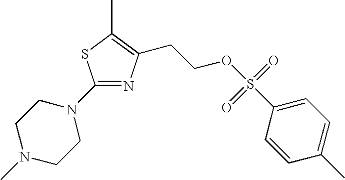

MS (ESI) m/z 396.1 (M+H)⁺.

### Toluene-4-sulfonic acid 2-[5-methyl-2-(4-phenyl-piperazin-1-yl)-thiazol-4-yl]-ethyl ester

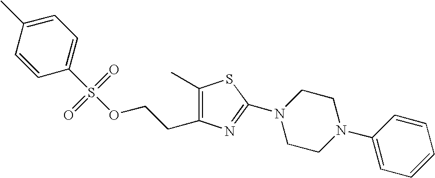

MS (ESI) m/z 458.1 (M+H)⁺.

### 2-(5-Methyl-2-phenyl-thiazol-4-yl)-ethanol

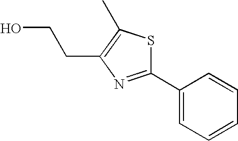

MS (ESI) m/z 220 (M+H)⁺.

### Toluene-4-sulfonic acid 2-(5-methyl-2-phenyl-thiazol-4-yl)-ethyl ester

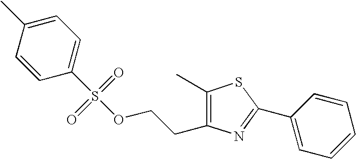

MS (ESI) m/z 374 (M+H)⁺.

### 2-(2-Biphenyl-4-yl-5-methyl-thiazol-4-yl)-ethanol

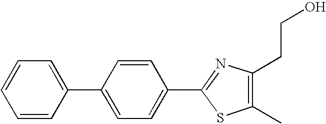

MS (ESI) m/z 296 (M+H)⁺.

### Toluene-4-sulfonic acid 2-(2-biphenyl-4-yl-5-methyl-thiazol-4-yl)-ethyl ester

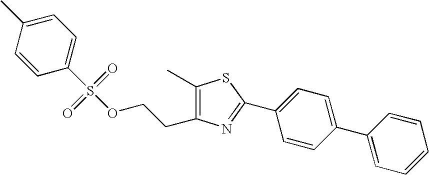

MS (ESI) m/z 450 (M+H)⁺.

### Toluene-4-sulfonic acid 2-(5-methyl-2-pyridin-2-ylthiazol-4-yl)ethyl ester

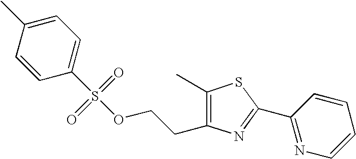

MS (ESI) m/z 375.1 (M+H)⁺.

### Toluene-4-sulfonic acid 2-(5-methyl-2-pyridin-3-ylthiazol-4-yl)ethyl ester

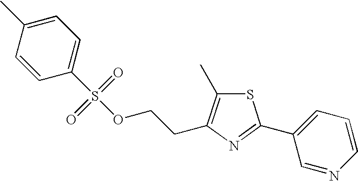

MS (ESI) m/z 375.1 (M+H)⁺.

### Toluene-4-sulfonic acid 2-(5-methyl-2-pyridin-4-ylthiazol-4-yl)ethyl ester

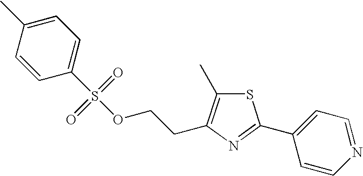

MS (ESI) m/z 375 (M+H)⁺.

### Toluene-4-sulfonic acid 2-[2-(2-methoxyethylamino)-5-methylthiazol-4-yl]ethyl ester

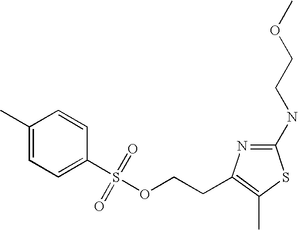

MS (ESI) m/z 371 (M+H)⁺.

### Toluene-4-sulfonic acid 2-(5-methyl-2-morpholin-4-yl-thiazol-4-yl)-ethyl ester

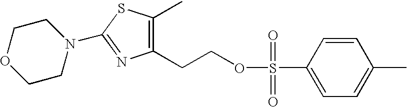

MS (ESI) m/z 383 (M+H)⁺.

### Preparation 10

### Toluene-4-sulfonic acid 2-(5-methyl-3-phenyl-pyrazol-1-yl)-ethyl ester

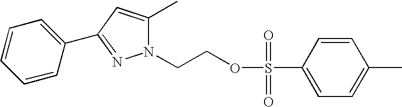

### Step A

### 5-Methyl-3-phenyl-1H-pyrazole

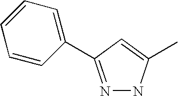

Hydrazine hydrate (9.0 mL, 99 mmol, 35 wt. % in H₂O; 0.64 equiv) is added to a solution of benzoylacetone (25.00 g, 154.1 mmol, 1 equiv) in ethanol (250 mL). After stirring 14 h, more hydrazine hydrate (8.0 mL, 88 mmol, 0.57 equiv) is added. After 2 h, the reaction solution is concentrated (95° C.) to give the title compound as a white solid (24.31 g, 99.7%). HRMS Calculated for C₁₀H₁₁N₂: m/Z 159.0922. Found: 159.0917.

### Step B

### 2-(5-Methyl-3-phenyl-pyrazol-1-yl)-ethanol

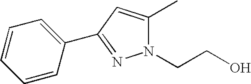

Sodium hydride (2.5 g, 1.5 g NaH, 62 mmol, 1.1 equiv) is added over a period of 3 min to a solution of 5-methyl-3-phenyl-1H-pyrazole (9.00 g, 56.9 mmol, 1 equiv) in DMF (90 mL) cooled to 0° C. in an ice bath. After stirring 15 min, ethylene carbonate (7.6 mL, 10 g, 110 mmol, 2.0 equiv) is added. The bath is removed, and the reaction mixture is stirred for 15 h. The mixture is treated with 4 M aq K₂CO₃ (90 mL), heated at reflux for 5 h, and diluted with H₂O (200 mL). After allowing the hot mixture to cool for 15 min, more H₂O (100 mL) and then hexanes (100 mL) are added. The mixture is shaken vigorously and then allowed to separate. Crystals formed and stayed with the top organic layer. The aqueous layer is separated, and the crystals are collected by vacuum filtration and washed with hexanes (2×50 mL). The crystals are dissolved in Et₂O/EtOAc (1:1; 200 mL), and the solution is dried (Na₂SO₄), filtered, and concentrated (75° C.) to give the title compound as an off-white crystalline solid (6.86 g, 59.6%).

HRMS Calculated for C₁₂H₁₅N₂O: m/z 203.1184. Found: 203.1168.

### Step C

### Toluene-4-sulfonic acid 2-(5-methyl-3-phenyl-pyrazol-1-yl)-ethyl ester

According to Preparation 9, Step E, 2-(5-methyl-3-phenyl-pyrazol-1-yl)-ethanol is converted into the title compound. MS (ESI) m/z 357 (M+H)⁺.

### Preparation 11

### 2-(3-Biphenyl-4-yl-5-methyl-pyrazol-1-yl)-ethanol

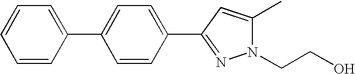

### Step A

### 5-Biphenyl-4-yl-3-methyl-1H-pyrazole

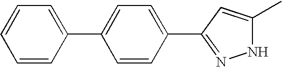

To a stirred mixture of NaH (1.98 g, 0.049 mol, 60% oil dispersion) in dry THF (30 mL) is added a suspension of diethoxyphosphorylacetone tosyl hydrazone (8.97 g, 0.024 mol; N Almirante Syn. Lett. 1999, 302.) in a mixture of THF (35 mL) and DMF (5.0 mL) dropwise over 15 min. The yellow suspension is stirred at 0-5° C. for 30 min and is treated with a 4-biphenyl carboxaldehyde (3.10 g, 0.0169 mol) in dry THF (30 mL) at 0-5° C. over 15 min. The orange solution is heated and stirred at reflux for 4 h and stirred at ambient temperature overnight. The mixture is poured into 5% aq. NaH₂PO₄ (350 mL) and extracted with EtOAc (2×200 mL). The organic layers are combined, washed with brine, dried (MgSO₄), filtered, and concentrated to a yellow semi-solid. This material is triturated with hot EtOAc (20 mL) and filtered. The solid is washed with EtOAc (2×10 mL) and dried under high vacuum to give the title compound (2.61 g, 47%): HRMS Calculated for C₁₆H₁₅N₂: m/z 235.1235. Found: 235.1230.

### Step B

### 2-(3-Biphenyl-4-yl-5-methyl-pyrazol-1-yl)-ethanol

The title compound is prepared from 5-biphenyl-4-yl-3-methyl-1H-pyrazole according to the Preparation 10, Step B. HRMS Calculated for C₁₈H₁₉N₂O: M/Z 279.1497. Found: 279.1496.

The following intermediate compounds are prepared by a substantially similar manner as described in Preparations 10 and 11.

### 2-[3-(4-Bromo-phenyl)-5-methyl-pyrazol-1-yl]-ethanol

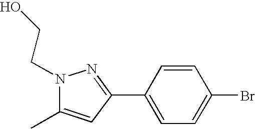

HRMS Calculated for Cl₂H₁₄BrN₂O: m/z 281.0289. Found: 281.0288.

### 3-Methyl-5-naphthalen-2-yl-1H-pyrazole

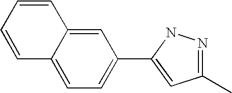

HRMS Calculated for C₁₄H₁₂N₂: m/z 208.1001. Found: 208.0981.

### 2-(5-Methyl-3-naphthalen-2-yl-pyrazol-1-yl)-ethanol

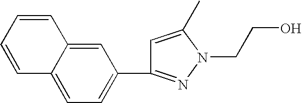

HRMS Calculated for C₁₆H₁₇N₂O: m/z 253.1341. Found: 253.1339.

### 3-Methyl-5-naphthalen-1-yl-1H-pyrazole

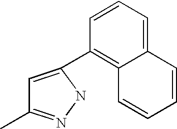

Anal Calculated for C₁₄H₁₂N₂: C, 80.74; H, 5.81; N, 13.45. Found: C, 80.93; H, 5.70; N, 13.42; mp 115-117° C.

### 2-(5-Methyl-3-naphthalen-1-yl-pyrazol-1-yl)-ethanol

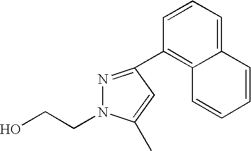

### Preparation 12

### 2-(2-Bromo-ethoxy)-6-methoxynaphthalene

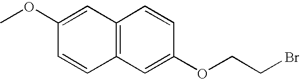

To a solution of 6-methoxynaphthalen-2-ol (1.07 g, 6.14 mmol) in DMF (4 mL) are added cesium carbonate (3.11 g, 9.55 mmol) and dibromoethane (2.5 mL, 29 mmol). The mixture is stirred and heated at 55° C. for 48 h. The reaction mixture is cooled, filtered, diluted with EtOAc, and washed with brine (2×30 mL). The organic layer is dried (Na₂SO₄) and concentrated. The crude product is purified using radial chromatography (2:98 to 25:75 EtOAc:Hex) to give the title compound as a white solid (0.52 g, 30%): ¹H NMR (400 MHz, CDCl₃) [symbol] 3.61 (t, J=6.1 Hz, 2H), 3.82 (s, 3H), 4.30 (t, J=6.4 Hz, 2H), 7.01-7.08 (m, 4H), 7.56 (dd, J=12.0, 9.0 Hz, 2H).

The following intermediate compounds are prepared by a substantially similar manner as described in Preparation 12.

### 6-(2-Bromoethoxy)-3-phenylbenzofuran

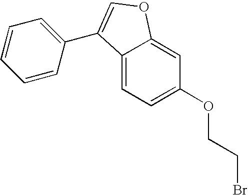

The above compound is prepared from 3-phenylbenzofuran-6-ol (see Bull. Soc. Chim. Fr., 942 (1962)). ¹H NMR (400 MHz, CDCl₃) [symbol] 3.60 (t, J=6.4 Hz, 2H), 4.28 (t, J=6.4 Hz, 2H), 6.88 (dd, J=8.8, 2.4 Hz, 1H), 7.00 (d, J=2.4 Hz, 1H), 7.26-7.30 (m, 1H), 7.36-7.44 (m, 2H), 7.52-7.56 (m, 2H), 7.63 (d, J=9.8 Hz, 2H).

### 4-(2-Bromoethoxy-1-phenoxybenzene

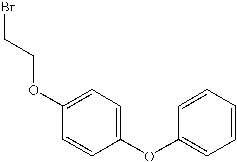

¹H NMR (400 MHz, CDCl₃) [symbol] 3.55 (t, J=6.4 Hz, 2H), 4.19 (t, J=6.1 Hz, 2H), 6.80-6.90 (m, 6H), 6.96 (t, J=7.3 Hz, 1H), 7.17-7.24 (m, 2H).

4-(3-Bromoethoxy)biphenyl: MS (ESI) m/z 295 (M+NH₃)⁺.

3-(2-Bromoethoxy)biphenyl: ¹H NMR (400 MHz, CDCl₃) δ 3.58 (t, J=6.4 Hz, 2H), 4.27 (t, J=6.4 Hz, 2H), 6.81 (dd, J=8.3, 2.4 Hz, 1H), 6.90 (d, J=8.8 Hz, 1H), 7.13 (dd, J=7.8, 1.0 Hz, 1H), 7.26-7.33 (m, 2H), 7.34-7.37 (m, 2H), 7.43-7.50 (m, 2H).

6-(4-Bromopropoxy)-3-phenylbenzofuran: ¹H NMR (400 MHz, CDCl₃) [symbol] 2.26-2.33 (m, 2H), 3.57 (t, J=6.4 Hz, 2H), 4.11 (t, J=5.9 Hz, 2H), 6.89 (dd, J=8.6, 2.2 Hz, 1H), 7.01 (d, J=2.0 Hz, 1H), 7.29 (t, J=7.6 Hz, 1H), 7.40 (t, J=7.6 Hz, 2H), 7.56 (d, J=6.8 Hz, 2H), 7.57-7.65 (m, 2H).

2-(4-Bromopropoxy)-6-methoxynaphthalene: ¹H NMR (400 MHz, CDCl₃) [symbol] 2.29 (t, J=6.1 Hz, 2H), 3.56 (t, J=6.4 Hz, 2H), 3.81 (4.11 (t, J=5.9 Hz, 2H), 7.01-7.14 (m, 4H), 7.52-7.57 (m, 2H).

3-(4-Bromopropoxy)biphenyl: ¹H NMR (400 MHz, CDCl₃) [symbol] 2.27 (t, J=6.1 Hz, 2H), 3.55 (t, J=6.6 Hz, 2H), 4.09 (t, J=5.9 Hz, 2H), 6.81 (dd, J=2.9, 1.0 Hz, 1H), 7.05 (t, J=2.0 Hz, 1H), 7.07 (t, J=2.0 Hz, 1H), 7.22-7.24 (m, 2H), 7.26-7.37 (m, 2H), 7.43-7.52 (m, 3H).

### 4-(3-Bromopropoxy)-biphenyl

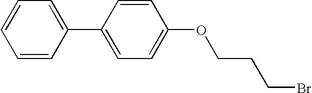

¹H-NMR (200.15 MHz, CDCl₃): [symbol] 7.57-7.29 (m, 7H), 6.98 (dd, 2H, J=6.72, 1.88), 4.15 (t, 2H, J=5.92), 3.62 (t, 2H, J=6.44), 2.34 (qn, 2H, J=5.92).

4-(3-Bromoproxy)-1-phenoxybenzene: ¹H-NMR (300 MHz, CDCl₃): [symbol] 7.3 (2H, m), 7.1 (2H, m), 7.0 (2H, m), 6.9 (2H, m), 4.1 (2H, m); 3.6 (2H, m); 2.3 (2H, m).

### 2-(4-Bromobutoxy)-6-methoxynaphthalene

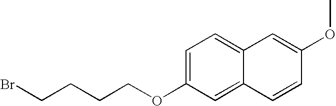

¹H NMR (400 MHz, CDCl₃) [symbol] 1.88-1.97 (m, 2H), 1.99-2.10 (m, 2H), 3.41-3.48 (m, 2H), 3.81 (s, 3H), 4.00 (t, J=5.9 Hz, 2H), 7.00-7.05 (m, 3H), 7.13-7.19 (m, 1H), 7.54 (t, J=8.1 Hz, 2H).

### 6-(4-Bromobutoxy)-3-phenylbenzofuran

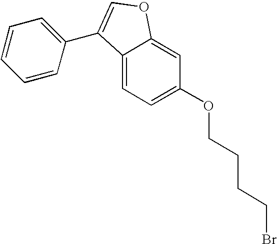

¹H NMR (400 MHz, CDCl₃) [symbol] 1.90-1.98 (m, 2H), 2.01-2.04 (m, 2H), 3.41-3.44 (m, 2H), 3.94-3.99 (m, 2H), 6.83-6.91 (m, 1H), 6.96-6.97 (m, 1H), 7.27-7.29 (m, 1H), 7.36-7.43 (m, 2H), 7.53-7.62 (m, 4H).

### 4-(4-Bromobutoxy)biphenyl

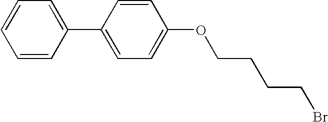

¹H NMR (400 MHz, CDCl₃) [symbol] 2.03 (t, J=6.8 Hz, 2H), 3.42 (t, J=6.6 Hz, 2H), 3.96 (t, J=6.5 Hz, 2H), 6.87 (d, J=7.8 Hz, 2H), 7.17-7.23 (m, 3H), 7.32 (t, J=7.6 Hz, 1H), 7.42-7.47 (m, 4H).

### 3-(4-Bromobutoxy)biphenyl

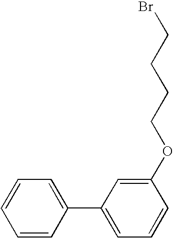

¹H NMR (400 MHz, CDCl₃) [symbol] 1.86-1.95 (m, 2H), 1.96-2.05 (m, 2H), 2.42 (t, J=6.6 Hz, 2H), 3.98 (t, J=6.1 Hz, 2H), 6.79 (dd, J=7.3, 2.0 Hz, 1H), 7.03-7.11 (m, 1H), 7.26 (t, J=7.8 Hz, 2H), 7.35 (t, J=7.8 Hz, 2H), 7.42-7.51 (m, 2H).

### 4-(4-Bromobutoxy)-1-phenoxybenzene

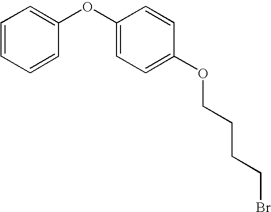

¹H NMR (400 MHz, CDCl₃) [symbol] 1.83-2.03 (m, 2H), 1.96-2.03 (m, 2H), 3.41 (t, J=6.6 Hz, 2H), 3.90 (t, J=6.5 Hz, 2H), 6.76-6.78 (m, 2H), 6.79-6.90 (m, 4H), 6.94-6.97 (m, 1H), 7.19-7.23 (m, 2H).

### Preparation 13

### 2-(4-Bromo-phenyl)-4-methyl-thiazole-5-carboxylic acid ethyl ester

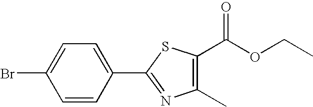

Dry 4-bromo-thioamide (3.4 g, 15 mmol) and ethyl 2-chloroacetoacetate (2.71 g, 16.4 mmol) are heated in ethanol (1000 mL) overnight. The cooled reaction is concentrated and purified by short path distillation. The fractions that contained pure product are concentrated to yield 1.5 g (30.6%) ester as a solid. MS (ES): 327 (M⁺+1).

### 4-Ethyl-2-(4-trifluoromethyl-phenyl)-thiazole-5-carboxylic acid ethyl ester

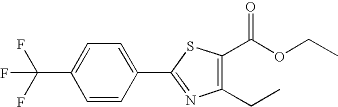

The title compound is prepared by a substantially similar manner as described in Preparation 13. MS (ES): 330 (M⁺+1).

### Preparation 14

### [4-Methyl-2-(4-trifluoromethyl-phenyl)-thiazol-5-yl]-methanol

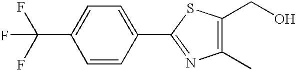

The title compound is prepared by a substantially similar manner as described in Preparation 13. The corresponding esters were reduced to the alcohols using the following example protocol.

A THF (60 mL) solution of 4-methyl-2-(4-trifluoromethyl-phenyl)-thiazole-5-carboxylic acid ethyl ester (14.9 g, 47.3 mmol) is cooled to 0° C. and a 1M LiAlH₄ (47.3 mL, 47.3 mmol) is added slowly. The reaction is warmed to rt slowly, and after stirring at rt for 2 h, TLC (15% EtOAc/hexane) showed that all the starting ester had been consumed. The reaction is cooled and carefully quenched with 2.4 mL water, 2.4 mL SN NaOH and 7 mL water. The light tan solid is filtered through celite and dried to give 7.70 g crude product. Recrystallization from methanol gave pure alcohol. MS (ES): 274 (M⁺+1).

The following intermediate compounds are prepared by a substantially similar manner as described in Preparations 13 and 14.

### (5-Methyl-2-phenyl-oxazol-4-yl)-methanol

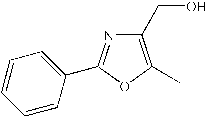

MS (ES): 190 (M⁺+1).

### [2-(4-Fluoro-phenyl)-5-methyl-oxazol-4-yl]-methanol

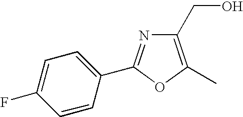

MS (ES): 208 (M⁺+1).

### [2-(4-Bromo-phenyl)-5-methyl-oxazol-4-yl]-methanol

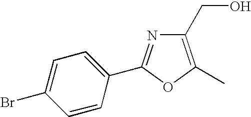

MS (ES): 269 (M⁺+1).

### [4-Methyl-2-(4-trifluoromethyl-phenyl)-oxazol-5-yl]-methanol

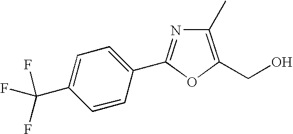

MS (ES): 258 (M⁺+1).

### [5-Methyl-2-(4-trifluoromethyl-phenyl)-thiazol-4-yl]-methanol

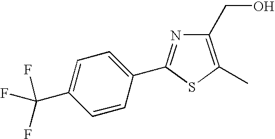

MS (ES): 274 (M⁺+1).

### 2-[5-Ethyl-2-(4-trifluoromethyl-phenyl)-thiazol-4-yl]-ethanol

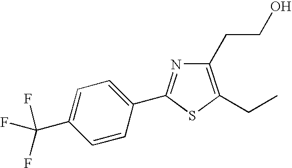

MS (ES): 302 (M⁺+1).

### [4-Methyl-2-(4-bromo-phenyl)-thiazol-5-yl]-methanol

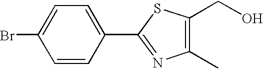

MS (ES): 284 (M⁺+1).

### [4-Ethyl-2-(4-trifluoromethyl-phenyl)-thiazol-5-yl]-methanol

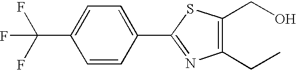

MS (ES): 288 (M⁺+1).

### Preparation 15

### 5-Chloromethyl-4-methyl-2-(4-trifluoromethyl-phenyl)-thiazole

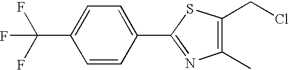

A solution of [4-methyl-2-(4-trifluoromethyl-phenyl)-thiazol-5-yl]-methanol (1.03 g, 3.75 mmol) and TEA (1.05 mL, 7.5 mmol) in methylene chloride (15 mL) is cooled to 0° C., and then MeSO₂Cl is added dropwise. After 2 hrs, TLC indicated that the reaction is not complete. Additional 10 mol % TEA and MeSO₂Cl are added. After additional 2 hrs, the mixture is diluted with methylene chloride and washed with sodium bicarbonate, water and brine, and then dried over sodium sulfate. Concentration affords the crude title compound, which is used for the next step without further purification. MS (ES): 292 (M⁺+1).

The following intermediate compounds are prepared by a substantially similar manner as described in Preparation 15.

### 5-Chloromethyl-4-methyl-2-(4-bromophenyl)-thiazole

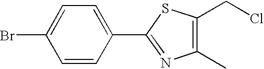

MS (ES): 303 (M⁺+1).

### 5-Chloromethyl-4-ethyl-2-(4-trifluoromethyl-phenyl)-thiazole

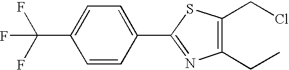

MS (ES): 306 (M⁺+1).

### Preparation 16

### 1-[4-Methyl-2-(4-trifluoromethyl-phenyl)-oxazol-5-yl]-ethanol

### Step A

### 2-(4-Trifluoromethyl-benzoylamino)-propionic acid methyl ester

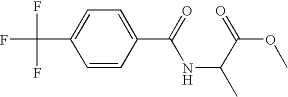

The compound of D,L-alanine methyl ester (18.5 g, 132 mmol), triethylamine (42 mL, 300 mmol) and dichloromethane (300 mL) are stirred in an ice/water bath. The compound of 4-(trifluoromethyl)benzoyl chloride (25 g, 120 mmol) is added dropwise, and the resulting mixture was allowed to stir for 20 hr at rt. 500 mL water and 100 mL 1M hydrochloric acid are successively added. The organic layer is separated, washed with 250 mL each of saturated sodium hydrogen carbonate, water, and brine, and then dried over anhydrous magnesium sulfate, filtered, and concentrated to 100 mL volume. The mixture is diluted with 200 mL hexanes and cooled to 0° C. for 1 hr. The white solids are filtered and dried under vacuum to afford about 26.5 g (80%) of the title compound. MS (ES): 276 (M⁺+1).

### Step B

### 2-(4-Trifluoromethyl-benzoylamino)-propionic acid

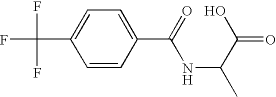

A mixture of 2-(4-tert-Butyl-benzoylamino)-propionic acid methyl ester (26.3 g, 95.6 mmol), 200 mL 1M sodium hydroxide, and 100 mL THF is stirred at rt for 20 hr. The resulting clear solution is cooled on an ice/water bath, and the pH is adjusted to 2 using conc. HCl. The product is extracted with three 250 mL portions of ethyl acetate. The combined extracts are washed with 100 mL each of water and brine, dried over anhydrous magnesium sulfate, filtered, and concentrated to afford about 24.6 g (95%) of the title compound a white solid. MS M⁺+1 260.

### Step C

### [4-Methyl-2-(4-trifluoromethyl-phenyl)-oxazol-5-yl]-methanol

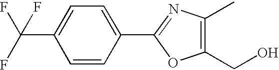

To a solution of 2-(4-trifluoromethyl-benzoylamino)-propionic acid (33.4 g, 128 mmol) is added oxalyl chloride (111 mL, 1.27 Mol) and 1 drop of DMF, and the solution is stirred overnight. The volatiles are removed in vacuo, and toluene (20 mL) is added. The toluene is then removed in vacuo. To the resultant crude oil is dissolve in 50 mL methylene chloride, cooled to 0° C. and TEA (27 mL, 192 mmol) is added followed by methanol (50 mL). After 3 hrs, the volatiles are removed in vacuo, and the crude oil is purified by flash column chromatography (20%-50% ethyl acetate/hexanes) to provide 12.6 g (35%) of 4-methyl-2-(4-trifluoromethyl-phenyl)-oxazole-5-carboxylic acid methyl ester. This ester (2.0 g, 7.0 mmol) is reduced to the alcohol by dissolution in THF (50 mL) and adding 4 eq. of LiBH₄ (0.610 g, 28.0 mmol) to provide 1.8 g (100%) of the title compound. MS M⁺+1 258.

### Step D

### 4-Methyl-2-(4-trifluoromethyl-phenyl)-oxazole-5-carbaldehyde

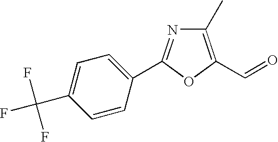

[4-Methyl-2-(4-trifluoromethyl-phenyl)-oxazol-5-yl]-methanol (2.42 g, 9.41 mmol) and 100 mL DCM are stirred at rt. Dess-Martin periodinane (8.0 g, 18.8 mmol) is added, and the resulting mixture is stirred 4 hr at rt. The mixture is diluted with 100 mL saturated sodium hydrogen carbonate. The organic layer is separated, washed with 50 mL each of water and brine, dried over anhydrous magnesium sulfate, filtered, and concentrated. The crude product is purified by silica gel chromatography eluting with a mixture of 8:2 hexanes:ethyl acetate to afford about 2.12 g (89%) of the title compound as a white solid. MS (M⁺+1) 256.

### Step F

### 1-[4-Methyl-2-(4-trifluoromethyl-phenyl)-oxazol-5-yl]-ethanol

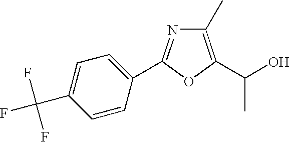

A solution of 4-methyl-2-(4-trifluoromethyl-phenyl)-oxazole-5-carbaldehyde (1.32 g, 5.16 mmol) and 50 mL THF is stirred at 0° C. Methyl magnesium bromide (2.2 mL, 6.71 mmol, 3M) is added dropwise, and the resulting mixture was allowed to stir at rt for 30 min. Additional amount of methyl magnesium bromide (1 mL, 3 mmol) is added, and the reaction is stirred an additional 1 hr at rt. The mixture is cooled on an ice/water bath, and aqueous ammonium chloride (10 mL) is added. The product is extracted with three 75 mL portions of ethyl acetate, and the combined extracts are dried over anhydrous magnesium sulfate, filtered and concentrated. The crude product is purified by silica gel chromatography eluting with a mixture of 1:1 hexanes:ethyl acetate to afford about 1.12 g (80%) of the title compound as an ivory solid. MS (M⁺+1) 272.

### Example 1

### General Procedure 1

### Synthesis of 5-[2-(2-Biphenyl-4-yl-5-methyl-oxazol-4-yl)-ethoxy]-8-(2-carboxy-ethyl)-3,4-dihydro-1H-isoquinoline-2-carboxylic acid propyl ester

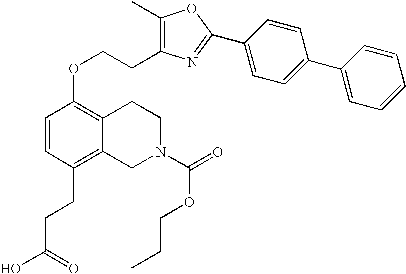

### Step A

### Coupling of Head and Tailpieces

### 5-[2-(2-Biphenyl-4-yl-5-methyl-oxazol-4-yl)-ethoxy]-8-(2-methoxycarbonyl-ethyl)-3,4-dihydro-1H-isoquinoline-2-carboxylic acid tert-butyl ester

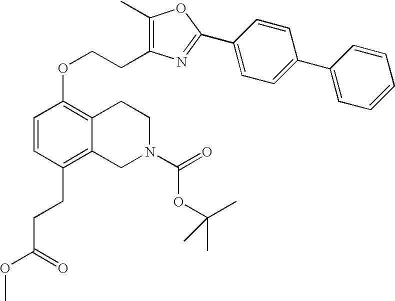

In a N₂ blanketed flask, 71.0 g (211.7 mmol) of 5-hydroxy-8-(2-methoxycarbonyl-ethyl)-3,4-dihydro-1H-isoquinoline-2-carboxylic acid tert-butyl ester (from Example 179), 119.3 g (275.2 mmol) of toluene-4-sulfonic acid 2-(2-biphenyl-4-yl-5-methyl-oxazol-4-yl)-ethyl ester (from Preparation 1), and 137.9 g (423.2 mmol) of cesium carbonate are combined in 625 mL of DMF. The mixture is stirred at 50° C. for 19 h until NMR and HPLC indicated complete reaction. After cooling to ambient temperature, the mixture is poured into 1500 mL of 50% saturated aq. NaCl. The aqueous layer is extracted with 3×600 mL of EtOAc. The combined organic layer is washed with 2×400 mL of a 10% aq. LiCl solution and dried over Na₂SO₄. Solvent is removed by rotary evaporation to afford crude 5-[2-(2-biphenyl-4-yl-5-methyl-oxazol-4-yl)-ethoxy]-8-(2-methoxycarbonyl-ethyl)-3,4-dihydro-1H-isoquinoline-2-carboxylic acid tert-butyl ester as 175.1 g of tan solids. The product is recrystallized by adding 500 mL of hexanes to a solution of the crude in 250 mL of EtOAc at reflux and allowing the solution to stir while slowly cooling to ambient temperature. The solids obtained by filtration are dried under vacuum to afford 81.3 g (64.4%) of the title compound as off-white solids. A second crop of 9.2 g (7.3%) of the title compound as white solids is obtained by chromatography followed by recrystallization of the filtrate.

### Step B

### Removal of Protecting Group, Tert-Boc

### 3-{5-[2-(2-Biphenyl-4-yl-5-methyl-oxazol-4-yl)-ethoxy]-1,2,3,4-tetrahydro-isoquinolin-8-yl}-propionic acid methyl ester

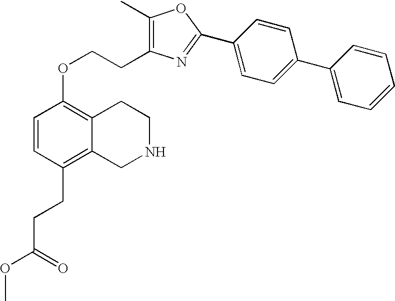

In a N₂ blanketed flask, compound of Step A (81.2 g, 136.08 mmol) is dissolved in 810 mL of DCM. To the stirring solution, 105 mL (155.4 g, 1362.9 mmol) of TFA is added over 30 minutes. After 21 h, when the reaction is completed, solvent is removed to afford 155 g of a dark viscous oil that is dissolved in 2 L of DCM and carefully washed with 2×800 mL of saturated aq. NaHCO₃ followed by 500 mL of 1; 1 saturated aq. NaHCO₃: brine. The organic phase is dried over Na₂SO₄. Solvent is removed to afford product as 70.2 g of tan solids (103.8%).

### Step C

### Nitrogen Acylation/Sulfonylation

### 5-[2-(2-Biphenyl-4-yl-5-methyl-oxazol-4-yl)-ethoxy]-8-(2-methoxycarbonyl-ethyl)-3,4-dihydro-1H-isoquinoline-2-carboxylic acid propyl ester

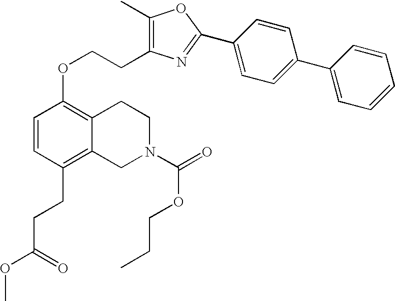

In a N₂ blanketed flask, 70.2 g (141.4 mmol) of compound obtained in Step B is dissolved in 1350 mL of CH₂Cl₂. To the solution is added 38 mL (27.6 g, 272.6 mmol) of TEA. The resultant solution is cooled to <10° C. and 23 mL (25.07 g, 204.6 mmol) of propyl chloroformate is added dropwise over twenty minutes with the temperature reaching 13° C. The solution is allowed to stir at ambient temperature for 15 h at which time the reaction is completed by HPLC indication. The mixture is washed with 2×450 mL of H₂O then brine and dried over Na₂SO₄. Solvent is removed to afford a viscous, brown oil which is eluted through 600 g of silica gel with 1:1 EtOAc:hexanes. The solvent is removed from fractions containing product to afford 67.7 g of product as slightly off-white solids (82.2%). MS (ESI) M/Z (M+H)+ 583.3.

### Step D

### Ester Hydrolysis Under Basic Conditions

### 5-[2-(2-Biphenyl-4-yl-5-methyl-oxazol-4-yl)-ethoxy]-8-(2-carboxy-ethyl)-3,4-dihydro-1H-isoquinoline-2-carboxylic acid propyl ester

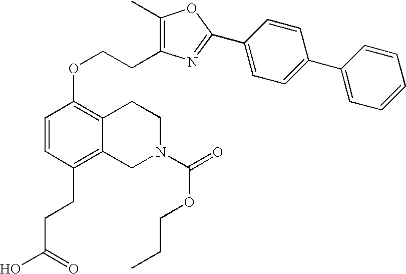

The methyl ester (67.6 g, 116.01 mmol) is suspended in 1 L of MeOH and heated to 35° C. To the solution, about 186 mL (930 mmol) of 5N NaOH is added over 15 min. The resulting white slurry is heated at 55° C. for 3 hours and then stirred at ambient temperature for 14 h. When the reaction is completed after an additional 90 min at 55° C., it is cooled to ambient temperature and filtered. The solids are washed with 5:1

MeOH:H₂O and dried under vacuum to provide 61.0 g of the title compound as white solids (89.1%). 1H NMR (250 MHz, CDCl₃) δ 8.15 (2H, d, J=7.4 Hz), 7.67 (2H, d, J=8.1 Hz), 7.55 (2H, d, J=7.4 Hz), 7.45-7.30 (3H, m), 6.91 (1H, d, J=7.4 Hz), 6.63 (1H, d, J=7.4 Hz), 4.48 (2H, d), 4.21 (2H, m), 3.95 (2H, t, J=5.6 Hz), 3.53 (2H, m), 3.07 (2H, m), 2.77 (2H, t, J=5.6 Hz), 2.65 (2H, t, J=5.6 Hz), 2.55 (2H, t, J=5.6 Hz), 2.37 (3H, s), 1.58 (2H, m), 0.88 (3H, t, J=5.6 Hz).

Examples 2 to 101 are prepared according to General Procedure 1 as described in Example 1 by using the appropriate tailpiece and appropriate sulfonyl chloride, chloroformate, or acylchloride group.

### Example 2

### 8-(2-Carboxy-ethyl)-5-{2-[5-methyl-2-(4-phenoxy-phenyl)-oxazol-4-yl]-ethoxy}-3,4-dihydro-1H-isoquinoline-2-carboxylic acid propyl ester

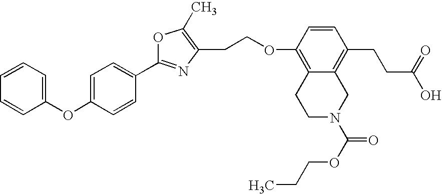

MS (ESI) M/Z (M+H)+ 585.

### Example 3

### ′8-(2-Carboxy-ethyl)-5-[2-(5-methyl-2-phenyl-oxazol-4-yl)-ethoxy]-3,4-dihydro-1H-isoquinoline-2-carboxylic acid tert-butyl ester

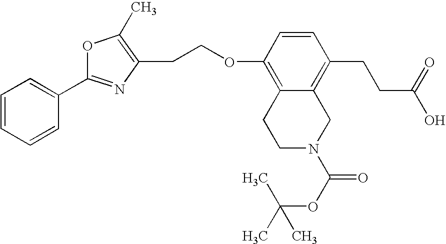

1H NMR (250 MHz, CDCl₃) δ 7.95-7.89 (2H, m), 7.38-7.30 (3H, m), 6.90 (1H, d, J=8.7 Hz), 6.61 (1H, d, J=8.7 Hz), 4.45 (2H, bs), 4.17 (2H, t, J=5.8 Hz), 3.55-3.48 (2H, m), 2.91 (2H, t, J=5.8 Hz), 2.81-2.49 (6H, m), 2.30 (3H, s), 1.41 (9H, s).

### Example 4

### ′3-{2-Methanesulfonyl-5-[2-(5-methyl-2-phenyl-oxazol-4-yl)-ethoxy]-1,2,3,4-tetrahydro-isoquinolin-8-yl}-propionic acid

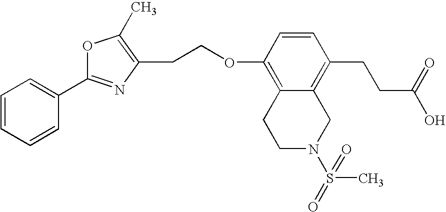

1H NMR (400 MHz, CDCl₃) δ 7.91-7.85 (2H, m), 7.38-7.30 (3H, m), 6.91 (1H, d, J=7.8 Hz), 6.62 (1H, d, J=7.8 Hz), 4.31 (2H, s), 4.15 (2H, t, J=5.9 Hz), 3.40 (2H, t, J=5.9 Hz), 2.92 (2H, t, J=5.9 Hz), 2.78-2.69 (5H, m), 2.55 (2H, t, J=5.9 Hz), 2.30 (3H, s).

### Example 5

### ′3-{2-Benzenesulfonyl-5-[2-(5-methyl-2-phenyl-oxazol-4-yl)-ethoxy]-1,2,3,4-tetrahydro-isoquinolin-8-yl}-propionic acid

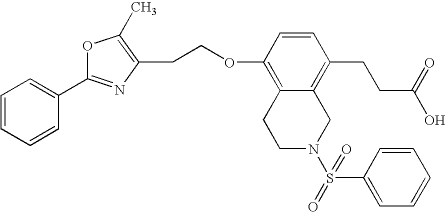

1H NMR (400 MHz, CDCl₃) δ 8.31-8.19 (2H, m), 7.80-7.71 (3H, m), 7.59-7.42 (5H, m), 6.83 (1H, d, J=7.9 Hz), 6.63 (1H, d, J=7.9 Hz), 4.28 (2H, s), 4.13-4.02 (2H, m), 3.28-3.12 (4H, m), 2.71-2.61 (4H, m), 2.52-2.42 (2H, m), 2.41 (3H, s).

### Example 6

### ′2-Methoxy-3-{5-[2-(5-methyl-2-phenyl-oxazol-4-yl)-ethoxy]-2-phenylmethanesulfonyl-1,2,3,4-tetrahydro-isoquinolin-8-yl}-propionic acid

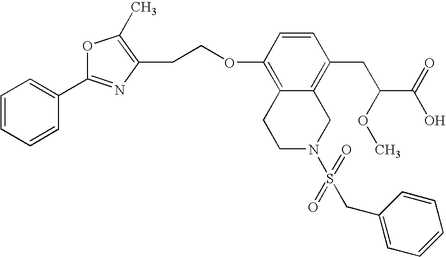

MS (ESI) M/Z (M+H)+ 591.

### Example 7

### 5-[2-(2-Biphenyl-4-yl-5-methyl-oxazol-4-yl)-ethoxy]-8-(2-carboxy-ethyl)-3,4-dihydro-1H-isoquinoline-2-carboxylic acid 2,2-dimethyl-propyl ester

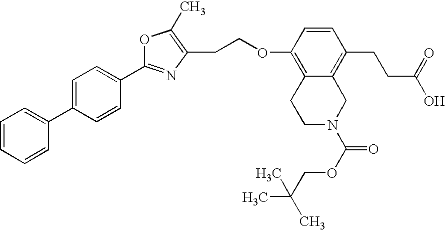

MS (ESI) M/Z (M+H)+ 597.

### Example 8

### 3-{2-(Biphenyl-4-sulfonyl)-5-[2-(2-biphenyl-4-yl-5-methyl-oxazol-4-yl)-ethoxy]-1,2,3,4-tetrahydro-isoquinolin-8-yl}-propionic acid

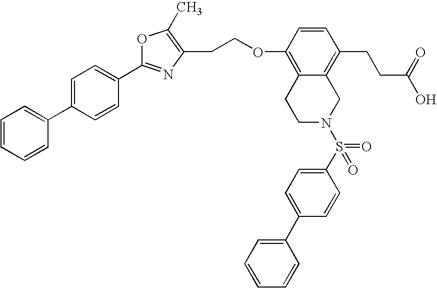

MS (ESI) M/Z (M+H)+ 699.

### Example 9

### 3-[5-[2-(2-Biphenyl-4-yl-5-methyl-oxazol-4-yl)-ethoxy]-2-(4-butoxy-benzenesulfonyl)-1,2,3,4-tetrahydro-isoquinolin-8-yl]-propionic acid

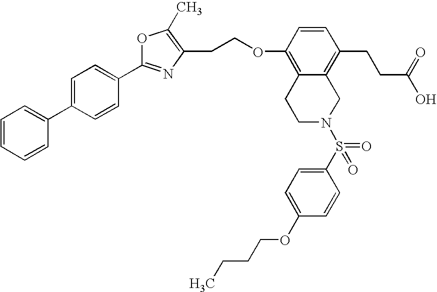

MS (ESI) M/Z (M+H)+ 695.

### Example 10

### 3-{2-(Biphenyl-4-sulfonyl)-5-[2-(5-methyl-2-phenyl-oxazol-4-yl)-ethoxy]-1,2,3,4-tetrahydro-isoquinolin-8-yl}-2-methoxy-propionic acid

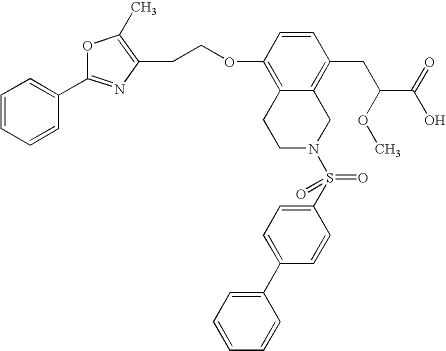

MS (ESI) M/Z (M+H)+ 653

### Example 11

### 8-(2-Carboxy-2-methoxy-ethyl)-5-[2-(5-methyl-2-phenyl-oxazol-4-yl)-ethoxy]-3,4-dihydro-1H-isoquinoline-2-carboxylic acid 2,2-dimethyl-propyl ester

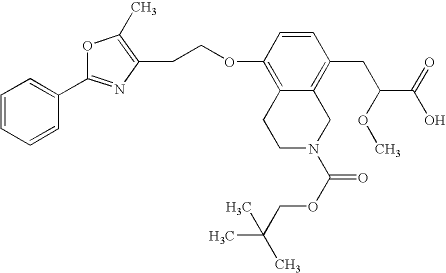

MS (ESI) M/Z (M+H)+ 551.

### Example 12

### 3-[5-[2-(2-Biphenyl-4-yl-5-methyl-oxazol-4-yl)-ethoxy]-2-(2,5-dichloro-thiophene-3-carbonyl)-1,2,3,4-tetrahydro-isoquinolin-8-yl]-propionic acid

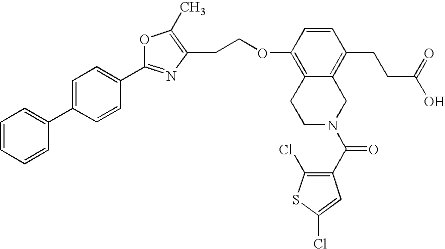

MS (ESI) M/Z (M+H)+ 661.

### Example 13

### 5-[2-(2-Biphenyl-4-yl-5-methyl-oxazol-4-yl)-ethoxy]-8-(2-carboxy-ethyl)-3,4-dihydro-1H-isoquinoline-2-carboxylic acid isobutylester

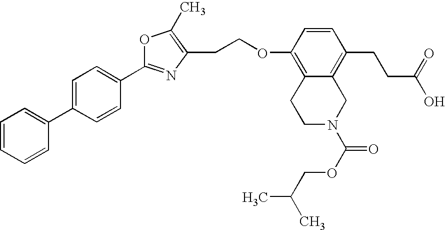

1H NMR (250 MHz, CDCl₃) δ 7.98 (2H, d, J=7.4 Hz), 7.60 (4H, m), 7.35 (3H, m), 6.93 (1H, d, J=7.4 Hz), 6.61 (1H, d, J=7.4 Hz), 4.52 (2H, s), 4.17 (2H, t, J=5.6 Hz), 3.83 (2H, d, J=6.2 Hz), 3.58 (2H, t, J=5.6 Hz), 2.95 (2H, t, J=5.6 Hz), 2.77 (2H, t, J=5.6 Hz), 2.67 (2H, t, 5.6 Hz), 2.55 (2H, t, J=5.6 Hz), 2.33 (3H, s), 1.88 (1H, m), 0.87 (6H, d, J=6.2 Hz).

### Example 14

### 3-{2-(2,5-Dichloro-thiophene-3-carbonyl)-5-[2-(5-methyl-2-phenyl-oxazol-4-yl)-ethoxy]-1,2,3,4-tetrahydro-isoquinolin-8-yl}-propionic acid

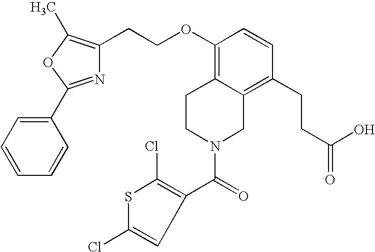

MS (ESI) M/Z (M+H)+ 586.

### Example 15

### 8-(2-Carboxy-ethyl)-5-[2-(5-methyl-2-phenyl-oxazol-4-yl)-ethoxy]-3,4-dihydro-1H-isoquinoline-2-carboxylic acid phenyl ester

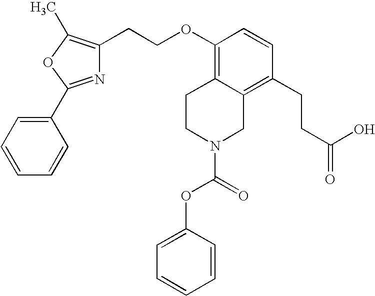

MS (ESI) M/Z (M+H)+ 527.

### Example 16

### 8-(2-Carboxy-ethyl)-5-[2-(5-methyl-2-phenyl-oxazol-4-yl)-ethoxy]-3,4-dihydro-1H-isoquinoline-2-carboxylic acid methyl ester

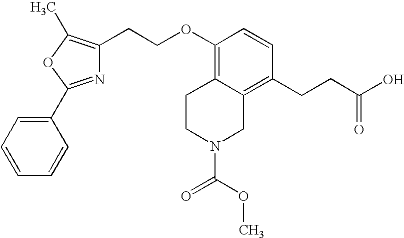

MS (ESI) M/Z (M+H)+ 465.

### Example 17

### 8-(2-Carboxy-ethyl)-5-[2-(5-methyl-2-phenyl-oxazol-4-yl)-ethoxy]-3,4-dihydro-1H-isoquinoline-2-carboxylic acid opropyl ester

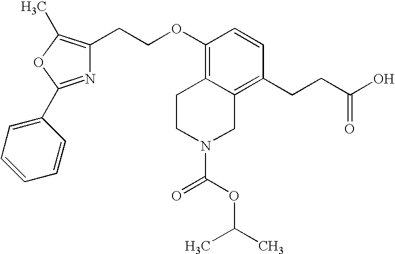

MS (ESI) M/Z (M+H)+ 493.

### Example 18

### 8-(2-Carboxy-ethyl)-5-[2-(5-methyl-2-phenyl-oxazol-4-yl)-ethoxy]-3,4-dihydro-1H-isoquinoline-2-carboxylic acid 2-chloro-ethyl ester

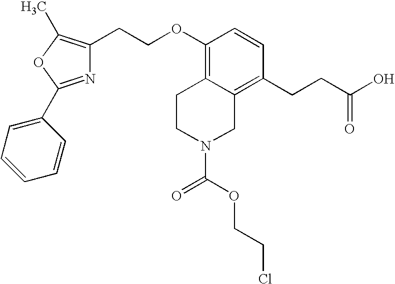

MS (ESI) M/Z (M+H)+ 514.

### Example 19

### 8-(2-Carboxy-ethyl)-5-[2-(5-methyl-2-phenyl-oxazol-4-yl)-ethoxy]-3,4-dihydro-1H-isoquinoline-2-carboxylic acid propyl ester

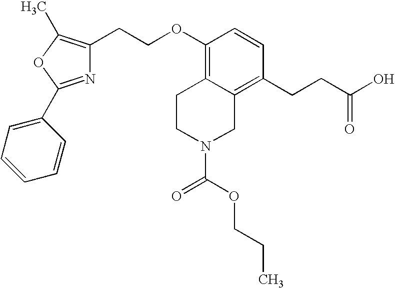

MS (ESI) M/Z (M+H)+ 493.

### Example 20

### 8-(2-Carboxy-ethyl)-5-[2-(-methyl-2-phenyl-oxazol-4-yl)-ethoxy]-3,4-dihydro-1H-isoquinoline-2-carboxylic acid butyl ester

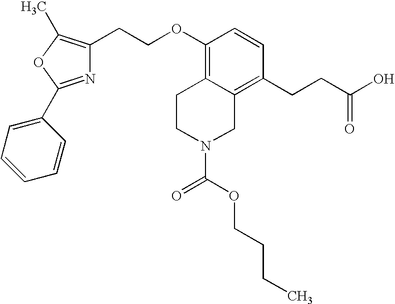

MS (ESI) M/Z (M+H)+ 507.

### Example 21

### 3-[5-[2-(5-Methyl-2-phenyl-oxazol-4-yl)-ethoxy]-2-(propane-1-sulfonyl)-1,2,3,4-tetrahydro-isoquinolin-8-yl]-propionic acid

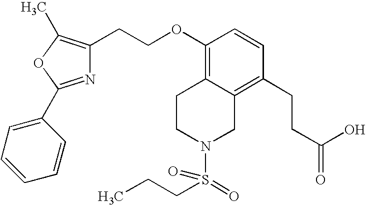

MS (ESI) M/Z (M+H)+ 513.

### Example 22

### 3-[5-[2-(5-Methyl-2-phenyl-oxazol-4-yl)-ethoxy]-2-(propane-2-sulfonyl)-1,2,3,4-tetrahydro-isoquinolin-8-yl]-propionic acid

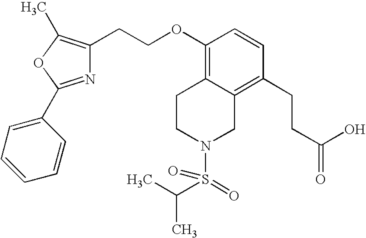

MS (ESI) M/Z (M+H)+ 513.

### Example 23

### 3-{2-Ethanesulfonyl-5-[2-(5-methyl-2-phenyl-oxazol-4-yl)-ethoxy]-1,2,3,4-tetrahydro-isoquinolin-8-yl}-propionic acid

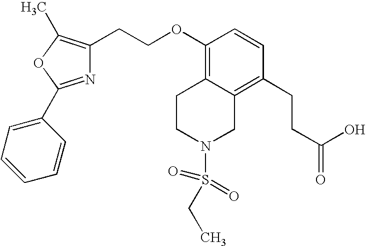

MS (ESI) M/Z (M+H)+ 499.

### Example 24

### 5-{2-[2-(4-Bromo-phenyl)-5-methyl-oxazol-4-yl]-ethoxy}-8-(2-carboxy-ethyl)-3,4-dihydro-1H-isoquinoline-2-carboxylic acid propyl ester

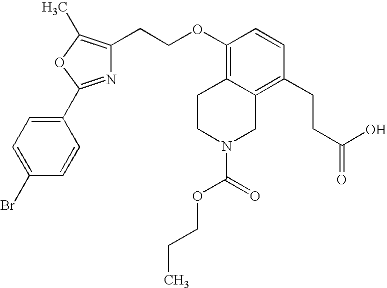

MS (ESI) M/Z (M+H)+ 573.

### Example 25

### 8-(2-Carboxy-ethyl)-5-[2-(5-methyl-2-thiophen-2-yl-oxazol-4-yl)-ethoxy]-3,4-dihydro-1H-isoquinoline-2-carboxylic acid propyl ester

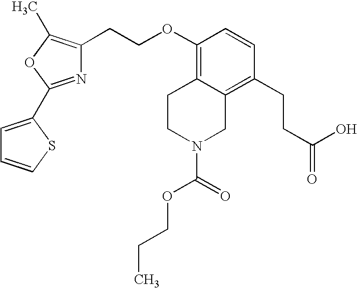

MS (ESI) M/Z (M+H)+ 499.

### Example 26

### 8-(2-Carboxy-ethyl)-5-[2-(2-cyclohexyl-5-methyl-oxazol-4-yl)-ethoxy]-3,4-dihydro-1H-isoquinoline-2-carboxylic acid propyl ester

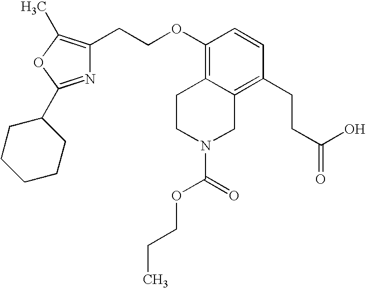

MS (ESI) M/Z (M+H)+ 499.

### Example 27

### 8-(2-Carboxy-ethyl)-5-{2-[5-methyl-2-(3-thiophen-2-yl-phenyl)-oxazol-4-yl]-ethoxy}-3,4-dihydro-1H-isoquinoline-2-carboxylic acid propyl ester

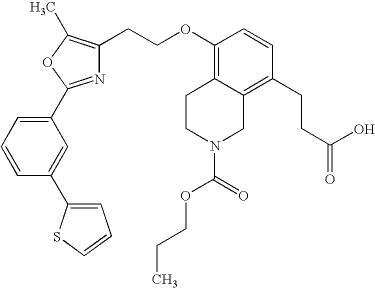

MS (ESI) M/Z (M+H)+ 575.

### Example 28

### 5-[2-(2-Biphenyl-3-yl-5-methyl-oxazol-4-yl)-ethoxy]-8-(2-carboxy-ethyl)-3,4-dihydro-1H-isoquinoline-2-carboxylic acid propyl ester

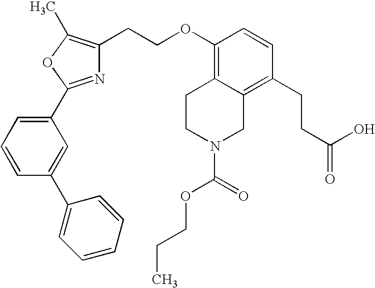

MS (ESI) M/Z (M+H)+ 569.

### Example 29

### 8-(2-Carboxy-ethyl)-5-{2-[5-methyl-2-(4-methyl-piperazin-1-yl)-thiazol-4-yl]-ethoxy}-3,4-dihydro-1H-isoquinoline-2-carboxylic acid propyl ester

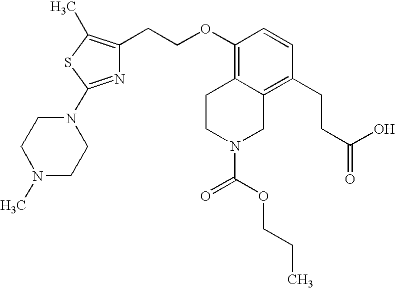

MS (ESI) M/Z (M+H)+ 531.

### Example 30

### 5-{2-[2-(4-Bromo-phenyl)-5-methyl-oxazol-4-yl]-ethoxy}-8-(2-carboxy-ethyl)-3,4-dihydro-1H-isoquinoline-2-carboxylic acid isobutylester

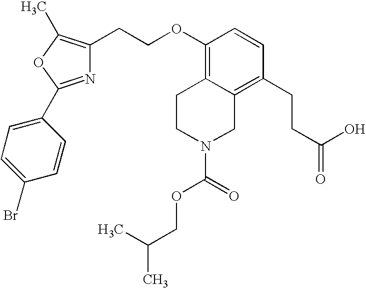

MS (ESI) M/Z (M+H)+ 586.

### Example 31

### 8-(2-Carboxy-ethyl)-5-[2-(5-methyl-2-thiophen-2-yl-oxazol-4-yl)-ethoxy]-3,4-dihydro-1H-isoquinoline-2-carboxylic acid isobutylester

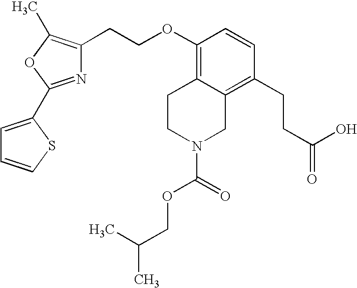

MS (ESI) M/Z (M+H)+ 513.

### Example 32

### 8-(2-Carboxy-ethyl)-5-{2-[5-methyl-2-(3-thiophen-2-yl-phenyl)-oxazol-4-yl]-ethoxy}-3,4-dihydro-1H-isoquinoline-2-carboxylic acid isobutylester

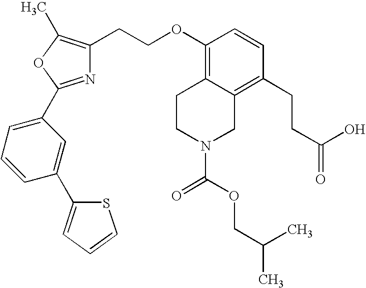

MS (ESI) M/Z (M+H)+ 589.

### Example 33

### 8-(2-Carboxy-ethyl)-5-[2-(2-cyclohexyl-5-methyl-oxazol-4-yl)-ethoxy]-3,4-hydro-1H-isoquinoline-2-carboxylic acid isobutylester

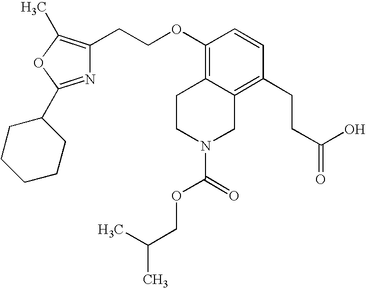

MS (ESI) M/Z (M+H)+ 513.

### Example 34

### 8-(2-Carboxy-ethyl)-5-{2-[5-methyl-2-(1-methyl-cyclohexyl)-oxazol-4-yl]-ethoxy}-3,4-dihydro-1H-isoquinoline-2-carboxylic acid isobutylester

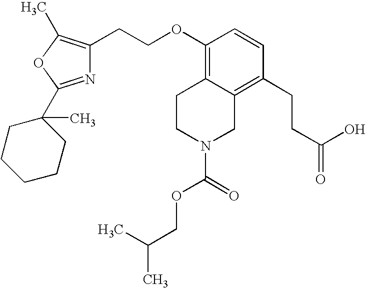

MS (ESI) M/Z (M+H)+ 527.

### Example 35

### 8-(2-Carboxy-ethyl)-5-[3-(4-phenoxy-phenoxy)-propoxy]-3,4-dihydro-1H-isoquinoline-2-carboxylic acid propyl ester

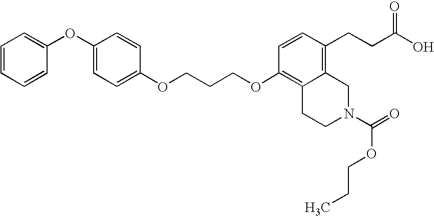

MS (ESI) M/Z (M+H)+ 534.

### Example 36

### 5-[3-(Biphenyl-4-yloxy)-propoxy]-8-(2-carboxy-ethyl)-3,4-dihydro-1H-isoquinoline-2-carboxylic acid propyl ester

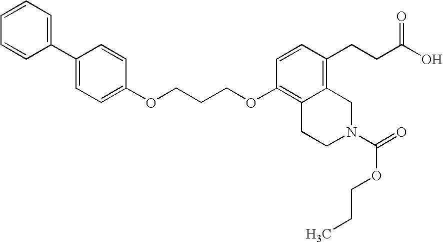

MS (ESI) M/Z (M+H)+ 518.

### Example 37

### 8-(2-Carboxy-ethyl)-5-[3-(4-phenoxy-phenoxy)-propoxy]-3,4-dihydro-1H-isoquinoline-2-carboxylic acid isobutylester

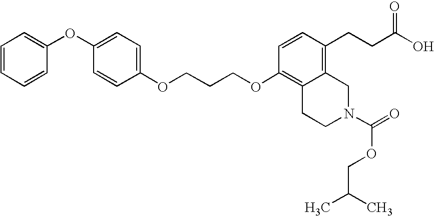

MS (ESI) M/Z (M+H)+ 548.

### Example 38

### 5-[3-(Biphenyl-4-yloxy)-propoxy]-8-(2-carboxy-ethyl)-3,4-dihydro-1H-isoquinoline-2-carboxylic acid isobutylester

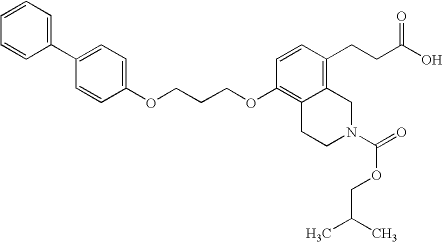

MS (ESI) M/Z (M+H)+ 532.

### Example 39

### 8-(2-Carboxy-ethyl)-5-[2-(5-methyl-2-pyridin-4-yl-thiazol-4-yl)-ethoxy]-3,4-dihydro-1H-isoquinoline-2-carboxylic acid propyl ester

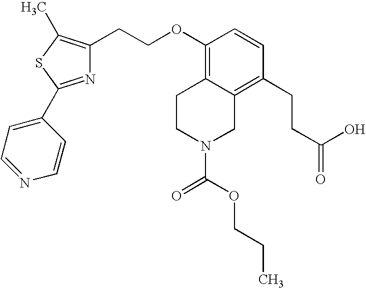

MS (ESI) M/Z (M+H)+ 510.

### Example 40

### 8-(2-Carboxy-ethyl)-5-[2-(5-methyl-2-phenyl-thiazol-4-yl)-ethoxy]-3,4-dihydro-1H-isoquinoline-2-carboxylic acid propyl ester

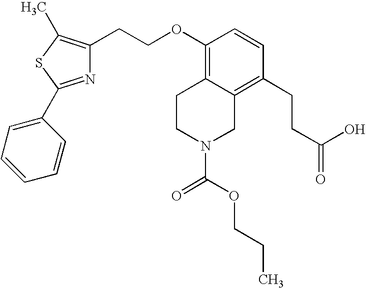

MS (ESI) M/Z (M+H)+ 509.

### Example 41

### 5-[2-(2-Benzyl-5-methyl-oxazol-4-yl)-ethoxy]-8-(2-carboxy-ethyl)-3,4-dihydro-1H-isoquinoline-2-carboxylic acid isobutylester

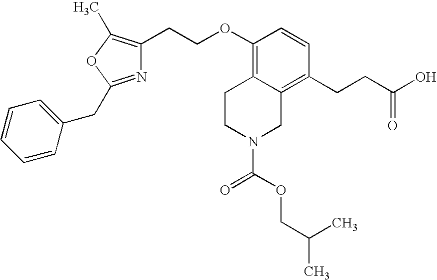

MS (ESI) M/Z (M+H)+ 521.

### Example 42

### 8-(2-Carboxy-ethyl)-5-{2-[5-methyl-2-(tetrahydro-pyran-4-yl)-oxazol-4-yl]-ethoxy}-3,4-dihydro-1H-isoquinoline-2-carboxylic acid isobutylester

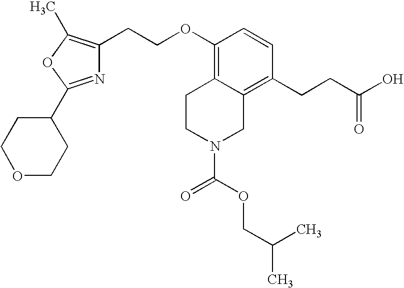

MS (ESI) M/Z (M+H)+ 515.

### Example 43

### 5-[2-(2-Benzo[b]thiophen-2-yl-5-methyl-oxazol-4-yl)-ethoxy]-8-(2-carboxy-ethyl)-3,4-dihydro-1H-isoquinoline-2-carboxylic acid isobutylester

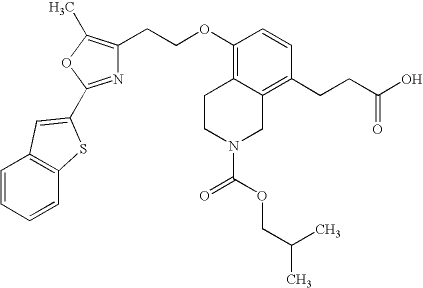

MS (ESI) M/Z (M+H)+ 563.

### Example 44

### 8-(2-Carboxy-ethyl)-5-[2-(5-methyl-3-phenyl-pyrazol-1-yl)-ethoxy]-3,4-dihydro-1H-isoquinoline-2-carboxylic acid isobutylester

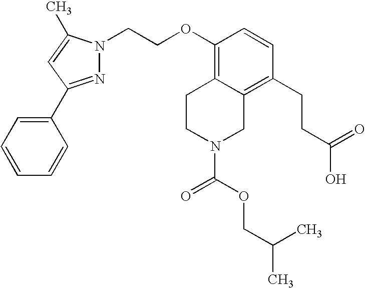

MS (ESI) M/Z (M+H)+ 506.

### Example 45

### 8-(2-Carboxy-ethyl)-5-{2-[5-methyl-2-(1-methyl-cyclohexyl)-oxazol-4-yl]-ethoxy}-3,4-dihydro-1H-isoquinoline-2-carboxylic acid propyl ester

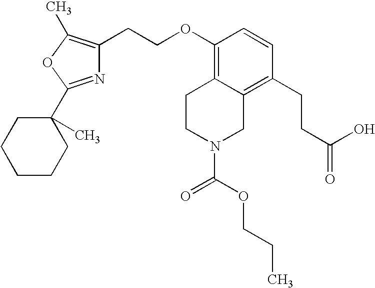

MS (ESI) M/Z (M+H)+ 513.

### Example 46

### 5-[2-(2-Benzyl-5-methyl-oxazol-4-yl)-ethoxy]-8-(2-carboxy-ethyl)-3,4-dihydro-1H-isoquinoline-2-carboxylic acid propyl ester

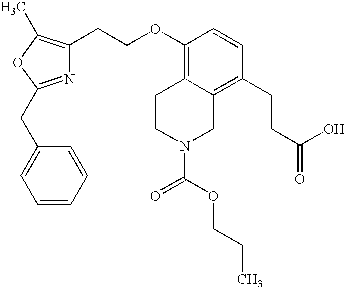

MS (ESI) M/Z (M+H)+ 507.

### Example 47

### 8-(2-Carboxy-ethyl)-5-[2-(5-methyl-2-phenethyl-oxazol-4-yl)-ethoxy]-3,4-dihydro-1H-isoquinoline-2-carboxylic acid propyl ester

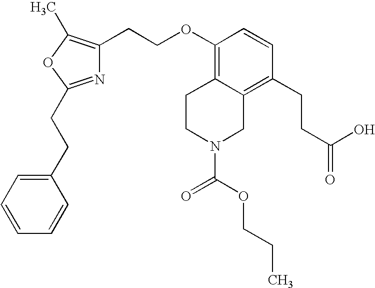

MS (ESI) M/Z (M+H)+ 521.

### Example 48

### 5-[2-(2-Benzo[b]thiophen-2-yl-5-methyl-oxazol-4-yl)-ethoxy]-8-(2-carboxy-ethyl)-3,4-dihydro-1H-isoquinoline-2-carboxylic acid propyl ester

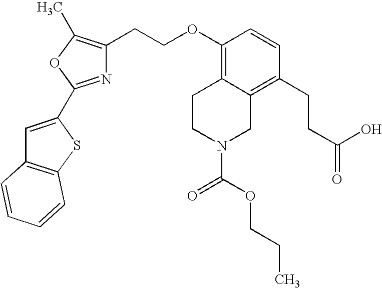

MS (ESI) M/Z (M+H)+ 549.

### Example 49

### -(2-Carboxy-ethyl)-5-[2-(5-methyl-3-phenyl-pyrazol-1-yl)-ethoxy]-3,4-dihydro-1H-isoquinoline-2-carboxylic acid propyl ester

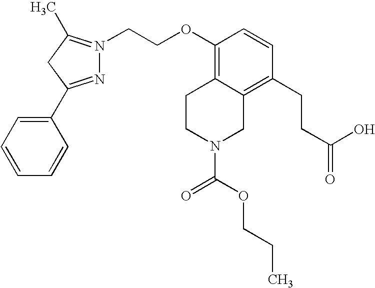

MS (ESI) M/Z (M+H)+ 492.

### Example 50

### 8-(2-Carboxy-ethyl)-5-{2-[5-methyl-2-(4-phenyl-piperazin-1-yl)-thiazol-4-yl]-ethoxy}-3,4-dihydro-1H-isoquinoline-2-carboxylic acid propyl ester

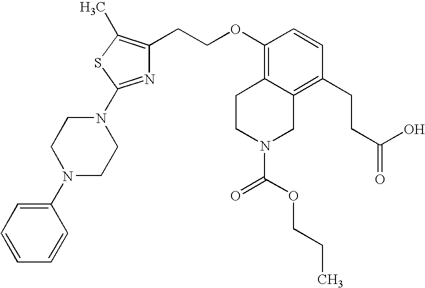

MS (ESI) M/Z (M+H)+ 593.

### Example 51

### 8-(2-Carboxy-ethyl)-5-{2-[5-methyl-2-(4-pyridin-3-yl-phenyl)-oxazol-4-yl]-ethoxy}-3,4-dihydro-1H-isoquinoline-2-carboxylic acid propyl ester

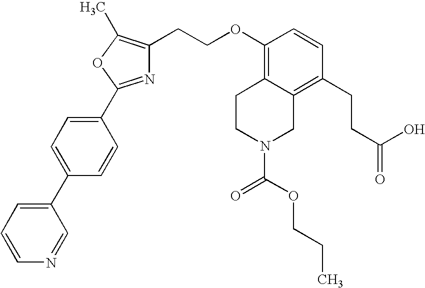

MS (ESI) M/Z (M+H)+ 570.

### Example 52

### ′3-{2-(3-Methoxy-benzoyl)-5-[2-(5-methyl-2-phenyl-oxazol-4-yl)-ethoxy]-1,2,3,4-tetrahydro-isoquinolin-8-yl}-propionic acid

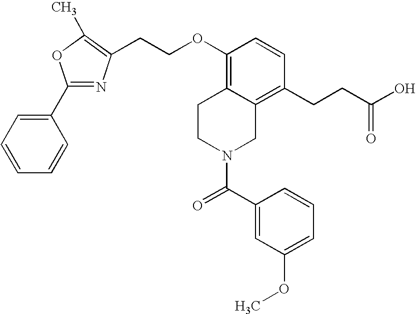

MS (ESI) M/Z (M+H)+ 541.

### Example 53

### ′8-(2-Carboxy-ethyl)-5-[2-(5-methyl-2-phenyl-oxazol-4-yl)-ethoxy]-3,4-dihydro-1H-isoquinoline-2-carboxylic acid benzyl ester

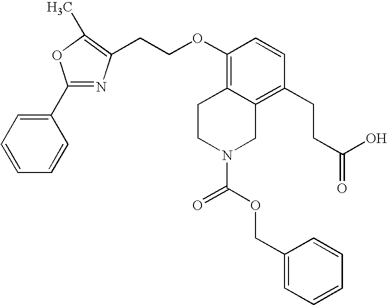

MS (ESI) M/Z (M+H)+ 541.

### Example 54

### ′3-{2-(Butane-1-sulfonyl)-5-[2-(5-methyl-2-phenyl-oxazol-4-yl)-ethoxy]-1,2,3,4-tetrahydro-isoquinolin-8-yl}-propionic acid

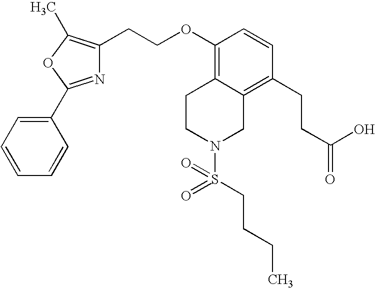

MS (ESI) M/Z (M+H)+ 527.

### Example 55

### ′3-[5-[2-(5-Methyl-2-phenyl-oxazol-4-yl)-ethoxy]-2-(4-nitro-benzenesulfonyl)-1,2,3,4-tetrahydro-isoquinolin-8-yl]-propionic acid

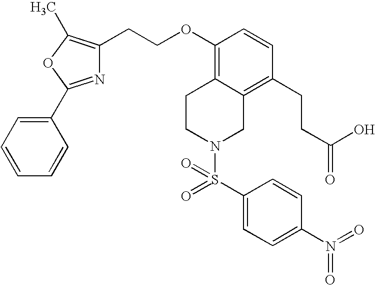

MS (ESI) M/Z (M+H)+ 592.

### Example 56

### ′3-{2-(4-Butoxy-benzenesulfonyl)-5-[2-(5-methyl-2-phenyl-oxazol-4-yl)-ethoxy]-1,2,3,4-tetrahydro-isoquinolin-8-yl}-propionic acid

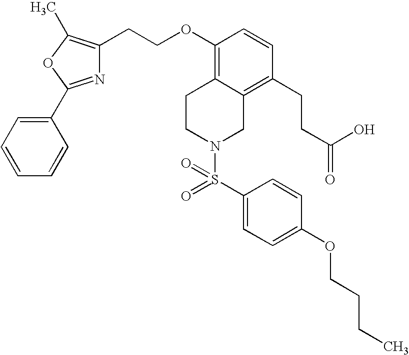

MS (ESI) M/Z (M+H)+ 619.

### Example 57

### ′3-{2-(5-Dimethylamino-naphthalene-1-sulfonyl)-5-[2-(5-methyl-2-phenyl-oxazol-4-yl)-ethoxy]-1,2,3,4-tetrahydro-isoquinolin-8-yl}-propionic acid

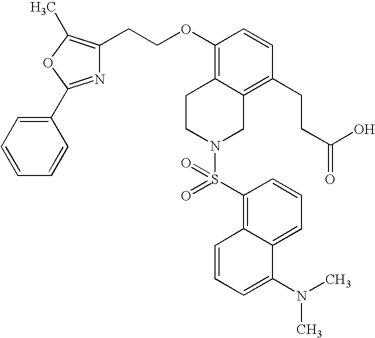

MS (ESI) M/Z (M+H)+ 640.

### Example 58

### ′3-{2-(Biphenyl-2-carbonyl)-5-[2-(5-methyl-2-phenyl-oxazol-4-yl)-ethoxy]-1,2,3,4-tetrahydro-isoquinolin-8-yl}-propionic acid

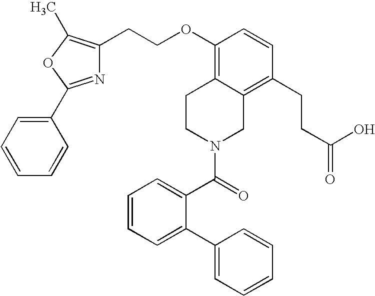

MS (ESI) M/Z (M+H)+ 587.

### Example 59

### ′3-{2-Diphenylacetyl-5-[2-(5-methyl-2-phenyl-oxazol-4-yl)-ethoxy]-1,2,3,4-tetrahydro-isoquinolin-8-yl}-propionic acid

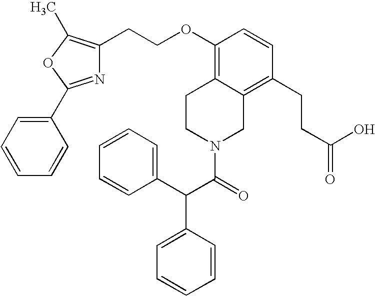

MS (ESI) M/Z (M+H)+ 601.

### Example 60

### ′3-{2-(Furan-2-carbonyl)-5-[2-(5-methyl-2-phenyl-oxazol-4-yl)-ethoxy]-1,2,3,4-tetrahydro-isoquinolin-8-yl}-propionic acid

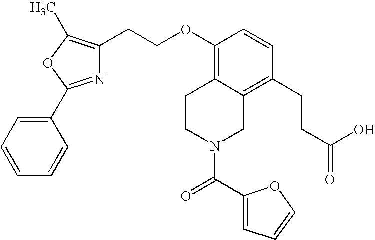

MS (ESI) M/Z (M+H)+ 501.

### Example 61

### ′3-[5-[2-(5-Methyl-2-phenyl-oxazol-4-yl)-ethoxy]-2-(pyridine-2-carbonyl)-1,2,3,4-tetrahydro-isoquinolin-8-yl]-propionic acid

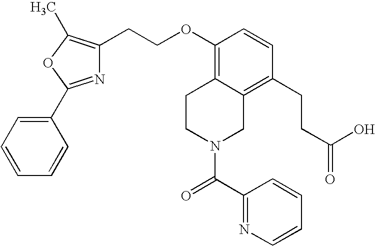

MS (ESI) M/Z (M+H)+ 512.

### Example 62

### ′3-[5-[2-(5-Methyl-2-phenyl-oxazol-4-yl)-ethoxy]-2-(pyrazine-2-carbonyl)-1,2,3,4-tetrahydro-isoquinolin-8-yl]-propionic acid

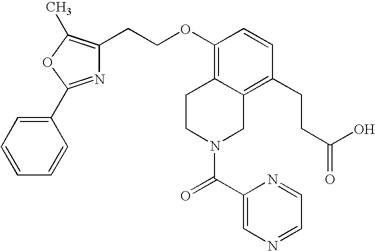

MS (ESI)/Z (M+H)+ 513.

### Example 63

### ′3-{2-(Biphenyl-4-sulfonyl)-5-[2-(5-methyl-2-phenyl-oxazol-4-yl)-ethoxy]-1,2,3,4-tetrahydro-isoquinolin-8-yl}-propionic acid

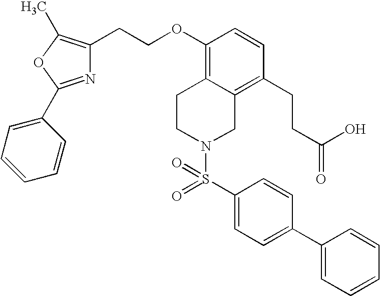

MS (ESI) M/Z (M+H)+ 623.

### Example 64

### ′3-[5-[2-(5-Methyl-2-phenyl-oxazol-4-yl)-ethoxy]-2-(4-trifluoromethyl-benzenesulfonyl)-1,2,3,4-tetrahydro-isoquinolin-8-yl]-propionic acid

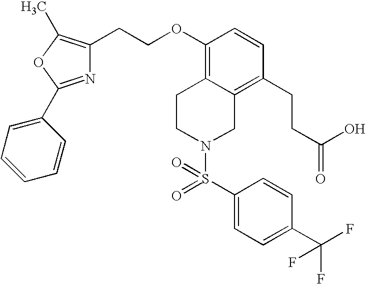

MS (ESI) M/Z (M+H)+ 615.

### Example 65

### ′3-{2-(4-Methoxy-benzenesulfonyl)-5-[2-(5-methyl-2-phenyl-oxazol-4-yl)-ethoxy]-1,2,3,4-tetrahydro-isoquinolin-8-yl}-propionic acid

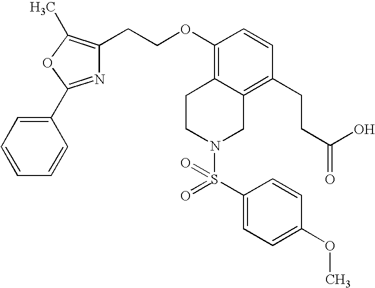

MS (ESI) M/Z (M+H)+ 577.

### Example 66

### ′8-(2-Carboxy-ethyl)-5-[2-(5-methyl-2-phenyl-oxazol-4-yl)-ethoxy]-3,4-dihydro-1H-isoquinoline-2-carboxylic acid 2,2-dimethyl-propyl ester

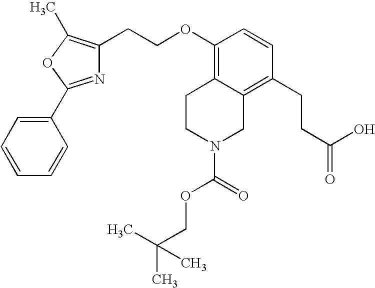

MS (ESI) M/Z (M+H)+ 521.

### Example 67

### ′8-(2-Carboxy-ethyl)-5-[2-(5-methyl-2-phenyl-oxazol-4-yl)-ethoxy]-3,4-dihydro-1H-isoquinoline-2-carboxylic acid cyclopentyl ester

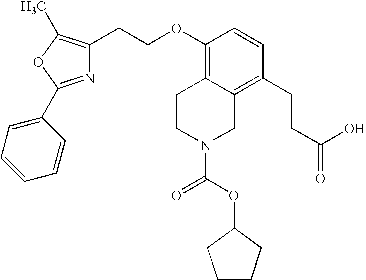

MS (ESI) M/Z (M+H)+ 519.

### Example 68

### ′3-[5-[2-(5-Methyl-2-phenyl-oxazol-4-yl)-ethoxy]-2-(2-phenoxy-acetyl)-1,2,3,4-tetrahydro-isoquinolin-8-yl]-propionic acid

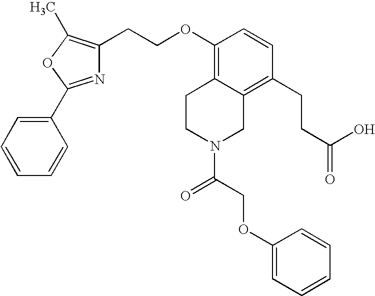

MS (ESI) M/Z (M+H)+ 541.

### Example 69

### 3-[5-[2-(5-Methyl-2-phenyl-oxazol-4-yl)-ethoxy]-2-(thiophene-2-sulfonyl)-1,2,3,4-tetrahydro-isoquinolin-8-yl]-propionic acid

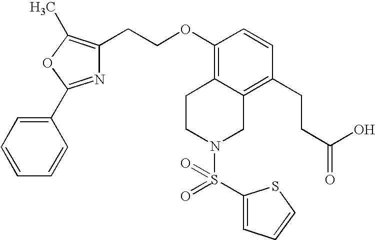

MS (ESI) M/Z (M+H)+ 553.

### Example 70

### 3-{2-(4-Chloro-benzenesulfonyl)-5-[2-(5-methyl-2-phenyl-oxazol-4-yl)-ethoxy]-1,2,3,4-tetrahydro-isoquinolin-8-yl}-propionic acid

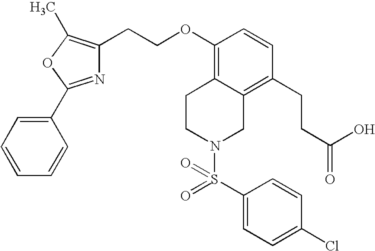

MS (ESI) M/Z (M+H)+ 582.

### Example 71

### 3-[5-[2-(5-Methyl-2-phenyl-oxazol-4-yl)-ethoxy]-2-(4-trifluoromethoxy-benzenesulfonyl)-1,2,3,4-tetrahydro-isoquinolin-8-yl]-propionic acid

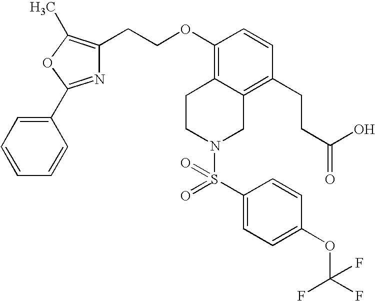

MS (ESI) M/Z (M+H)+ 631.

### Example 72

### 3-[5-[2-(5-Methyl-2-phenyl-oxazol-4-yl)-ethoxy]-2-(naphthalene-2-sulfonyl)-1,2,3,4-tetrahydro-isoquinolin-8-yl]-propionic acid

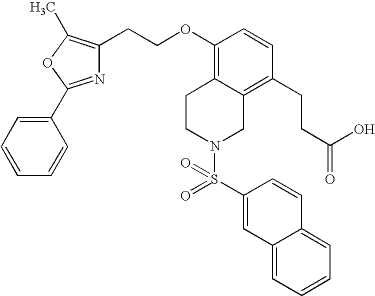

MS (ESI) M/Z (M+H)+ 597.

### Example 73

### 3-[5-[2-(5-Methyl-2-phenyl-oxazol-4-yl)-ethoxy]-2-(3-methylsulfanyl-propionyl)-1,2,3,4-tetrahydro-isoquinolin-8-yl]-propionic acid

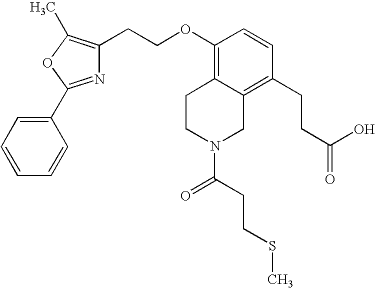

MS (ESI) M/Z (M+H)+ 509.

### Example 74

### 3-{2-(4-Methoxy-benzoyl)-5-[2-(5-methyl-2-phenyl-oxazol-4-yl)-ethoxy]-1,2,3,4-tetrahydro-isoquinolin-8-yl}-propionic acid

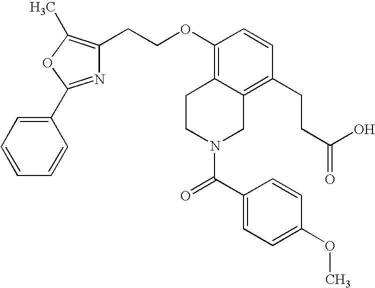

MS (ESI) M/Z (M+H)+ 541.

### Example 75

### 8-(2-Carboxy-ethyl)-5-[2-(5-methyl-2-phenyl-oxazol-4-yl)-ethoxy]-3,4-dihydro-1H-isoquinoline-2-carboxylic acid 2,2,2-trichloro-1,1-dimethyl-ethyl ester

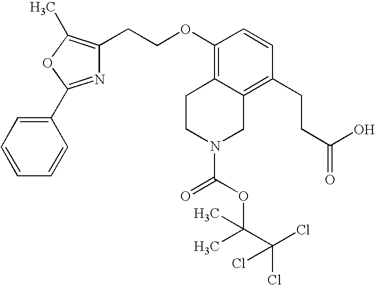

MS (ESI) M/Z (M+H)+ 611

### Example 76

### 3-{2-(3,5-Dimethoxy-benzoyl)-5-[2-(5-methyl-2-phenyl-oxazol-4-yl)-ethoxy]-1,2,3,4-tetrahydro-isoquinolin-8-yl}-propionic acid

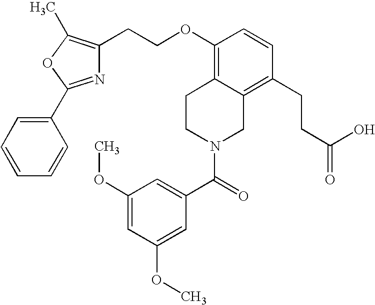

MS (ESI) M/Z (M+H)+ 571.

### Example 77

### 8-(2-Carboxy-ethyl)-5-[2-(5-methyl-2-phenyl-oxazol-4-yl)-ethoxy]-3,4-dihydro-1H-isoquinoline-2-carboxylic acid prop-2-ynyl ester

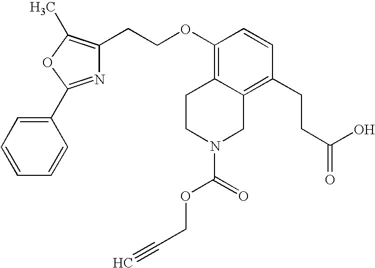

MS (ESI) M/Z (M+H)+ 489.

### Example 78

### 3-{2-(3,4-Dichloro-benzenesulfonyl)-5-[2-(2-methyl-5-phenyl-furan-3-yl)-ethoxy]-1,2,3,4-tetrahydro-isoquinolin-8-yl}-propionic acid

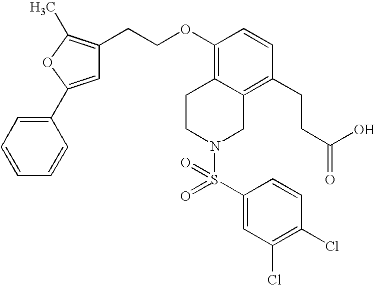

MS (ESI) M/Z (M+H)+ 615.

### Example 79

### 3-{2-(3,5-Dichloro-benzenesulfonyl)-5-[2-(2-methyl-5-phenyl-furan-3-yl)-ethoxy]-1,2,3,4-tetrahydro-isoquinolin-8-yl}-propionic acid

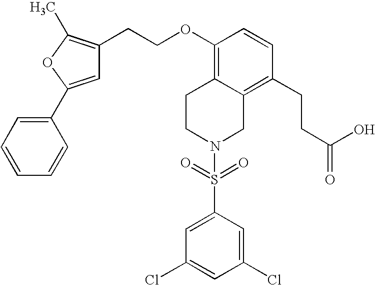

MS (ESI) M/Z (M+H)+ 615.

### Example 80

### 3-{2-(3-Methyl-3H-imidazole-4-sulfonyl)-5-[2-(2-methyl-5-phenyl-furan-3-yl)-ethoxy]-1,2,3,4-tetrahydro-isoquinolin-8-yl}-propionic acid

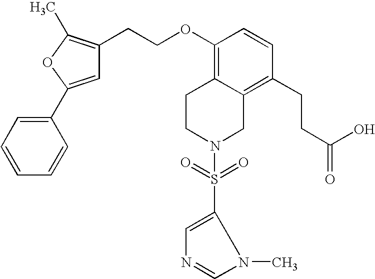

MS (ESI) M/Z (M+H)+ 550.

### Example 81

### 3-{2-(3,5-Dimethyl-oxazole-4-sulfonyl)-5-[2-(2-methyl-5-phenyl-furan-3-yl)-ethoxy]-1,2,3,4-tetrahydro-isoquinolin-8-yl}-propionic acid

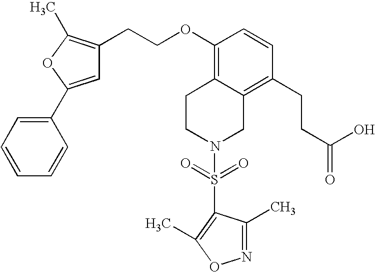

MS (ESI) M/Z (M+H)+ 565.

### Example 82

### 3-{2-(5-Chloro-3-methyl-benzo[b]thiophene-2-sulfonyl)-5-[2-(2-methyl-5-phenyl-furan-3-yl)-ethoxy]-1,2,3,4-tetrahydro-isoquinolin-8-yl}-propionic acid

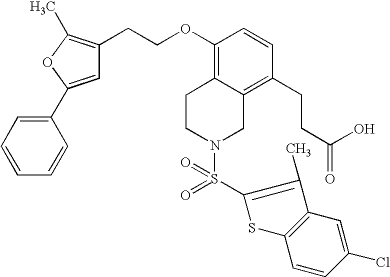

### Example 83

### 3-{2-(4-Methanesulfonyl-benzenesulfonyl)-5-[2-(2-methyl-5-phenyl-furan-3-yl)-ethoxy]-1,2,3,4-tetrahydro-isoquinolin-8-yl}-propionic acid

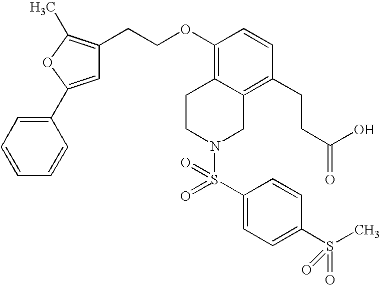

MS (ESI) M/Z (M+H)+ 624.

### Example 84

### 3-{2-(3,4-Dimethoxy-benzenesulfonyl)-5-[2-(2-methyl-5-phenyl-furan-3-yl)-ethoxy]-1,2,3,4-tetrahydro-isoquinolin-8-yl}-propionic acid

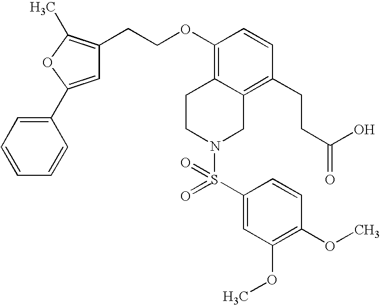

MS (ESI) M/Z (M+H)+ 606.

### Example 85

### 3-[5-[2-(2-Methyl-5-phenyl-furan-3-yl)-ethoxy]-2-(2-naphthalen-1-yl-ethanesulfonyl)-1,2,3,4-tetrahydro-isoquinolin-8-yl]-propionic acid

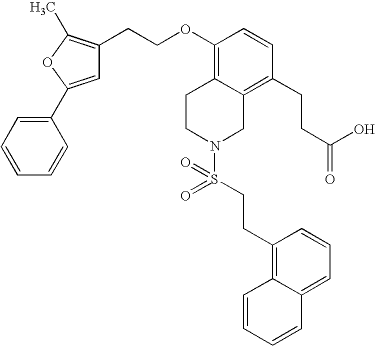

MS (ESI) M/Z (M+H)+ 625.

### Example 86

### 3-[5-[2-(5-Methyl-2-phenyl-oxazol-4-yl)-ethoxy]-2-(octane-1-sulfonyl)-1,2,3,4-tetrahydro-isoquinolin-8-yl]-propionic acid

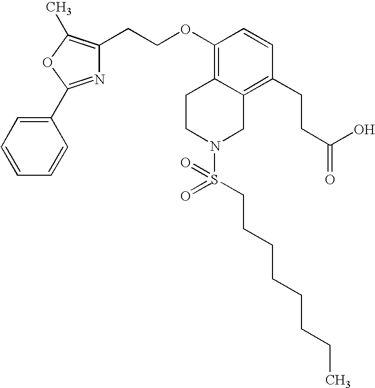

MS (ESI) M/Z (M+H)+ 583.

### Example 87

### 3-{2-(4-Ethyl-benzenesulfonyl)-5-[2-(5-methyl-2-phenyl-oxazol-4-yl)-ethoxy]-1,2,3,4-tetrahydro-isoquinolin-8-yl}-propionic acid

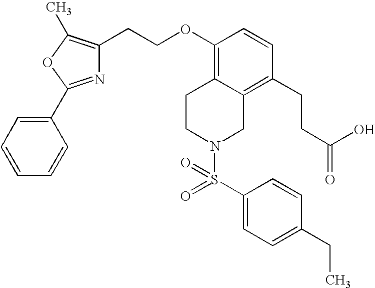

MS (ESI) M/Z (M+H)+ 575.

### Example 88

### 3-{2-(4-opropyl-benzenesulfonyl)-5-[2-(5-methyl-2-phenyl-oxazol-4-yl)-ethoxy]-1,2,3,4-tetrahydro-isoquinolin-8-yl}-propionic acid

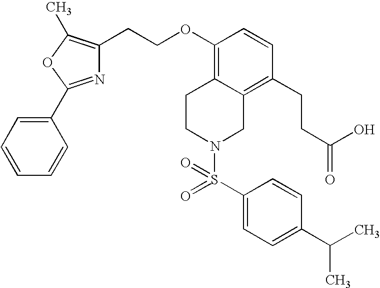

MS (ESI) M/Z (M+H)+ 589.

### Example 89

### 3-[5-[2-(5-Methyl-2-phenyl-oxazol-4-yl)-ethoxy]-2-(4-propyl-benzenesulfonyl)-1,2,3,4-tetrahydro-isoquinolin-8-yl]-propionic acid

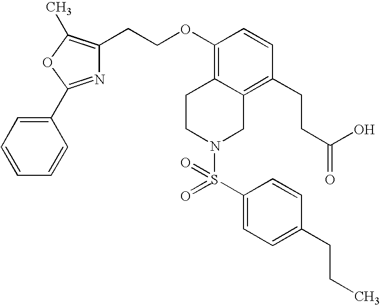

MS (ESI) M/Z (M+H)+ 589.

### Example 90

### 3-[5-[2-(5-Methyl-2-phenyl-oxazol-4-yl)ethoxy]-2-(4-pentyl-benzenesulfonyl)-1,2,3,4-tetrahydro-isoquinolin-8-yl]-propionic acid

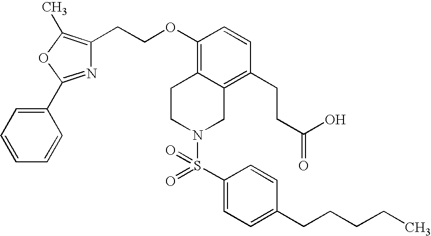

MS (ESI) M/Z (M+H)+ 617.

### Example 91

### 3-{2-[4-(1,1-Dimethyl-propyl)-benzenesulfonyl]-5-[2-(5-methyl-2-phenyl-oxazol-4-yl)-ethoxy]-1,2,3,4-tetrahydro-isoquinolin-8-yl}-propionic acid

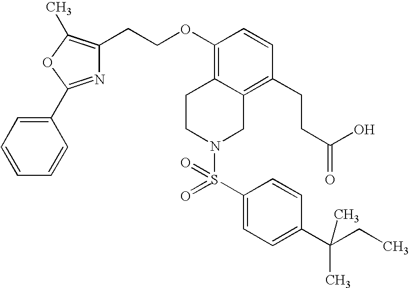

MS (ESI) M/Z (M+H)+ 617.

### Example 92

### 8-(2-Carboxy-ethyl)-5-[2-(5-methyl-2-phenyl-oxazol-4-yl)-ethoxy]-3,4-dihydro-1H-isoquinoline-2-carboxylic acid 2,2,2-trichloro-ethyl ester

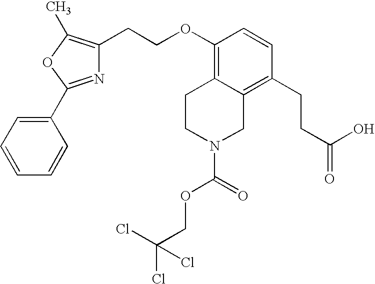

MS (ESI) M/Z (M+H)+ 583.

### Example 93

### 8-(2-Carboxy-ethyl)-5-[2-(5-methyl-2-phenyl-oxazol-4-yl)-ethoxy]-3,4-dihydro-1H-isoquinoline-2-carboxylic acid 4-nitro-benzyl ester

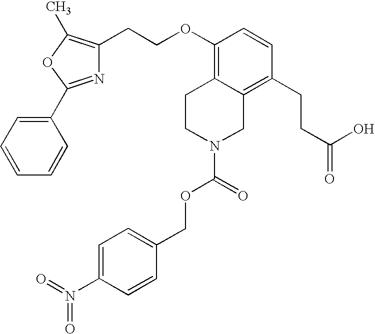

MS (ESI) M/Z (M+H)+ 586.

### Example 94

### 8-(2-Carboxy-ethyl)-5-[2-(5-methyl-2-phenyl-oxazol-4-yl)-ethoxy]-3,4-dihydro-1H-isoquinoline-2-carboxylic acid 2-methoxy-ethyl ester

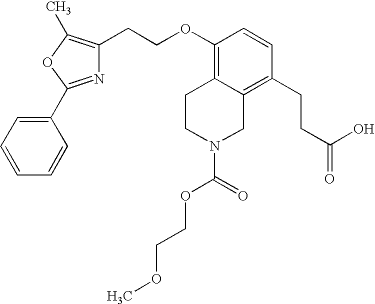

MS (ESI) M/Z (M+H)+ 509.

### Example 95

### 3-[5-[2-(5-Methyl-2-phenyl-oxazol-4-yl)-ethoxy]-2-(5-pyridin-3-yl-thiophene-2-sulfonyl)-1,2,3,4-tetrahydro-isoquinolin-8-yl]-propionic acid

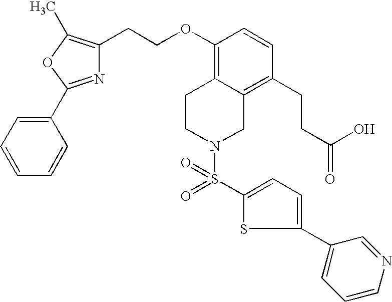

MS (ESI) M/Z (M+H)+ 630.

### Example 96

### 3-{2-(2,4-Dichloro-thiophene-3-sulfonyl)-5-[2-(5-methyl-2-phenyl-oxazol-4-yl)-ethoxy]-1,2,3,4-tetrahydro-isoquinolin-8-yl}-propionic acid

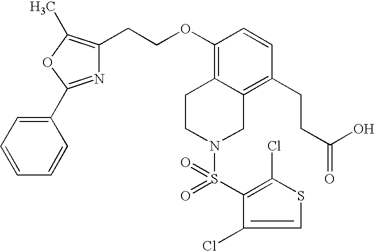

MS (ESI) M/Z (M+H)+ 622.

### Example 97

### 3-{2-(4-tert-Butyl-benzenesulfonyl)-5-[2-(5-methyl-2-phenyl-oxazol-4-yl)-ethoxy]-1,2,3,4-tetrahydro-isoquinolin-8-yl}-propionic acid

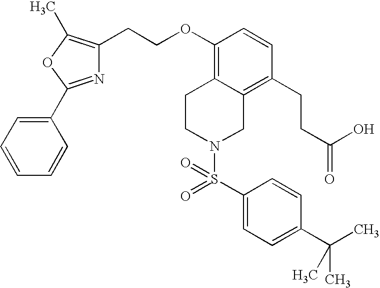

MS (ESI) M/Z (M+H)+ 603.

### Example 98

### 8-(2-Carboxy-ethyl)-5-[2-(5-methyl-2-phenyl-oxazol-4-yl)-ethoxy]-3,4-dihydro-1H-isoquinoline-2-carboxylic acid isobutylester

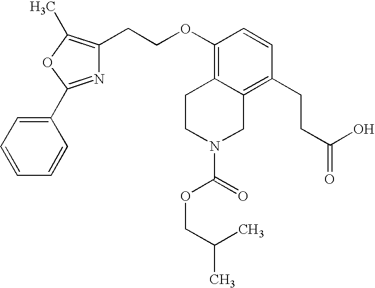

MS (ESI) M/Z (M+H)+ 507.

### Example 99

### 8-(2-Carboxy-ethyl)-5-[2-(5-methyl-2-phenyl-oxazol-4-yl)-ethoxy]-3,4-dihydro-1H-isoquinoline-2-carboxylic acid 2-ethyl-hexyl ester

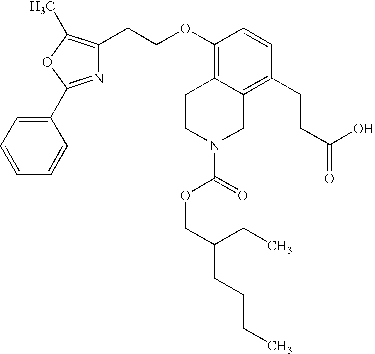

MS (ESI) M/Z (M+H)+ 563.

### Example 100

### 8-(2-Carboxy-ethyl)-5-[2-(5-methyl-2-phenyl-oxazol-4-yl)-ethoxy]-3,4-dihydro-1H-isoquinoline-2-carboxylic acid hexyl ester

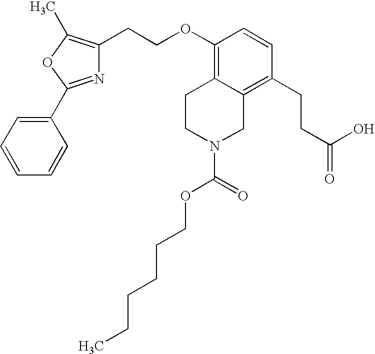

MS (ESI) M/Z (M+H)+ 535.

### Example 101

### 3-{2-(5-Benzenesulfonyl-thiophene-2-sulfonyl)-5-[2-(5-methyl-2-phenyl-oxazol-4-yl)-ethoxy]-1,2,3,4-tetrahydro-isoquinolin-8-yl}-propionic acid

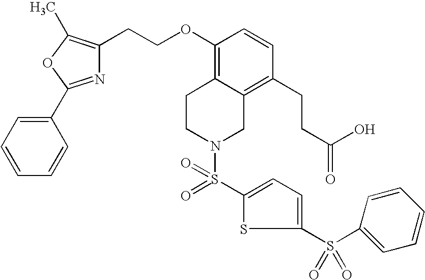

MS (ESI) M/Z (M+H)+ 693.

### Example 102

### General Procedure 2

### Arylation of the Nitrogen

### 3-[5-[2-(5-Methyl-2-phenyl-oxazol-4-yl)-ethoxy]-2-(4-trifluoromethyl-phenyl)-1,2,3,4-tetrahydro-isoquinolin-8-yl]-propionic acid

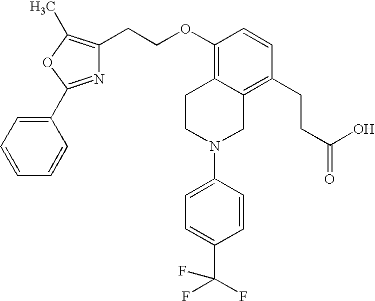

The headpiece and tailpiece are coupled and the product deprotected according to the procedure General Procedure 1 steps A and B as described in Example 1.

The compound of 3-{5-[2-(5-methyl-2-phenyl-oxazol-4-yl)-ethoxy]-1,2,3,4-tetrahydro-isoquinolin-8-yl}-propionic acid (40 mg, 0.095 mmol) is mixed with 4-trifluoromethyl phenylchloride (19.7 mg, 0.11 mmol), sodium t-butoxide (12.8 mg, 0.13 mmol), 2-(dicyclohexylphosphino)biphenyl (10 mg, 0.028 mmol) and Pd₂(dba)₃ (13 mg, 0.014 mmol) in DME (4 mL) in a seal tube at rt. The mixture is bubbled with N₂ for 5 minutes, stirred at 100° C. for 16 h, and then cooled, washed with water and diluted with EtOAc. The organic layer is dried (MgSO₄), concentrated and chromatographed (silica gel; hexane/EtOAc: 2:1) to afford an oil (21 mg, 40%) as the ester. The ester in MeOH (3 mL) is hydrolyzed with 3.0 N aqueous NaOH at 60° C. for 3 h. The mixture is acidified with 3.0 N HCl, extracted with CH₂Cl₂ and concentrated to afford the title compound. ¹H NMR (CDCl₃, 250 MHz) δ 2.40 (s, 3H), 2.66 (m, 2H), 2.90 (m, 4H), 3.00 (m, 2H), 3.58 (m, 2H), 4.35 (m, 2H), 4.44 (s, 2H), 6.75 (d, J=6.0 Hz, 1H), 7.00 (m, 2H), 7.04 (d, J=6.0 Hz, 1H), 7.42 (m, 3H), 7.52 (m, 2H), 8.02 (m, 2H).

Examples 103 to 122 are prepared according to General Procedure 1 and General Procedure 2 as described in Example 1 and Example 102.

### Example 103

### 3-[5′-[2-(5-Methyl-2-phenyl-oxazol-4-yl)-ethoxy]-2-(4-propionyl-phenyl)-1,2,3,4-tetrahydro-isoquinolin-8-yl]-propionic acid

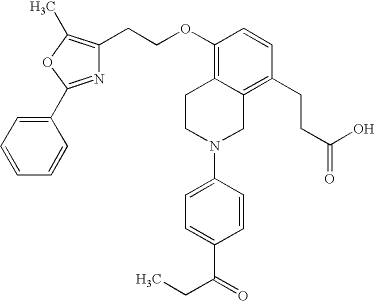

MS (ESI) M/Z (M+H)+ 539.

### Example 104

### 3-{2-(4-Acetyl-phenyl)-5-[2-(5-methyl-2-phenyl-oxazol-4-yl)-ethoxy]-1,2,3,4-tetrahydro-isoquinolin-8-yl}-propionic acid

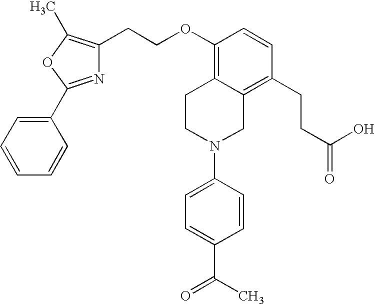

MS (ESI) M/Z (M+H)+ 525.

### Example 105

### 3-{2-(4-Methanesulfonyl-phenyl)-5-[2-(5-methyl-2-phenyl-oxazol-4-yl)-ethoxy]-1,2,3,4-tetrahydro-isoquinolin-8-yl}-propionic acid

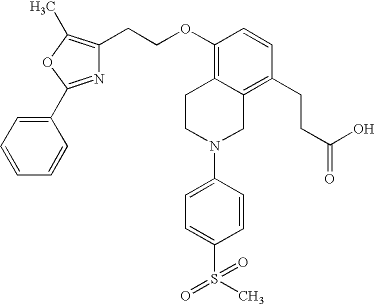

MS (ESI) M/Z (M+H)+ 561.

### Example 106

### 3-{2-(3,5-B-trifluoromethyl-phenyl)-5-[2-(5-methyl-2-phenyl-oxazol-4-yl)-ethoxy]-1,2,3,4-tetrahydro-isoquinolin-8-yl}-propionic acid

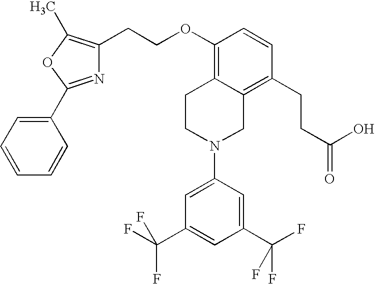

MS (ESI) M/Z (M+H)+ 619.

### Example 107

### 3-{2-(3-Fluoro-5-uoromethyl-phenyl)-5-[2-(5-methyl-2-phenyl-oxazol-4-yl)-ethoxy]-1,2,3,4-tetrahydro-isoquinolin-8-yl}-propionic acid

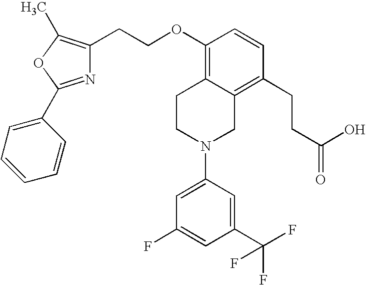

MS (ESI) M/Z (M+H)+ 569.

### Example 108

### Sodium; 3-[5-[2-(5-methyl-2-phenyl-oxazol-4-yl)-ethoxy]-2-(4-trifluoromethyl-phenyl)-1,2,3,4-tetrahydro-isoquinolin-8-yl]-propionate

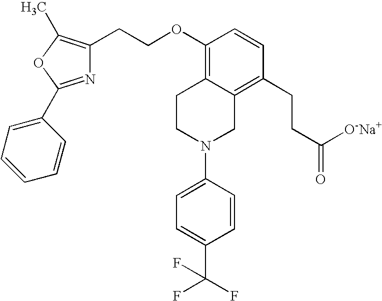

MS (ESI) M/Z (M+H)+ 551.

### Example 109

### 3-{2-(4-Dimethylamino-phenyl)-5-[2-(5-methyl-2-phenyl-oxazol-4-yl)-ethoxy]-1,2,3,4-tetrahydro-isoquinolin-8-yl}-propionic acid

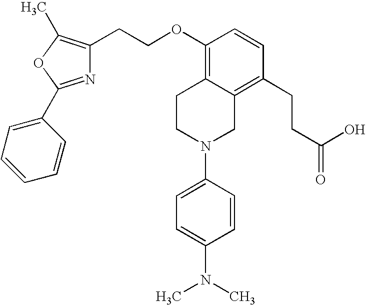

MS (ESI) M/Z (M+H)+ 526.

### Example 110

### 3-{2-(3,5-Difluoro-phenyl)-5-[2-(5-methyl-2-phenyl-oxazol-4-yl)-ethoxy]-1,2,3,4-tetrahydro-isoquinolin-8-yl}-propionic acid

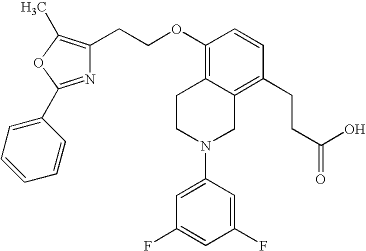

MS (ESI) M/Z (M+H)+ 519.

### Example 111

### 3-[5-[2-(5-Methyl-2-phenyl-oxazol-4-yl)-ethoxy]-2-(3,4,5-trifluoro-phenyl)-1,2,3,4-tetrahydro-isoquinolin-8-yl]-propionic acid

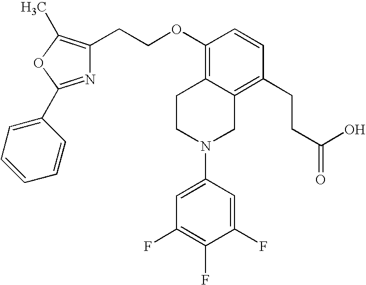

MS (ESI) M/Z (M+H)+ 537.

### Example 112

### 3-{2-(4-Ethylsulfanyl-phenyl)-5-[2-(5-methyl-2-phenyl-oxazol-4-yl)-ethoxy]-1,2,3,4-tetrahydro-isoquinolin-8-yl}-propionic acid

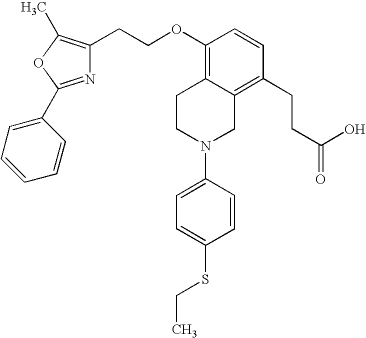

MS/ES+543.

### Example 113

### Sodium; 3-[5-[2-(5-methyl-2-morpholin-4-yl-thiazol-4-yl)-ethoxy]-2-(4-trifluoromethyl-phenyl)-1,2,3,4-tetrahydro-isoquinolin-8-yl]-propionate

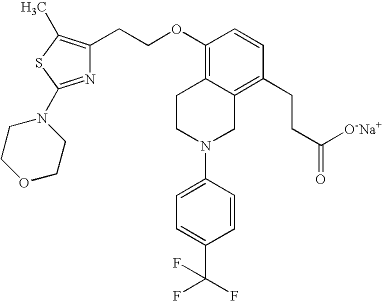

MS (ESI) M/Z (M+H)+ 576.

### Example 114

### 3-[5-[2-(2-Biphenyl-4-yl-5-methyl-oxazol-4-yl)-ethoxy]-2-(4-trifluoromethyl-phenyl)-1,2,3,4-tetrahydro-isoquinolin-8-yl]-propionic acid

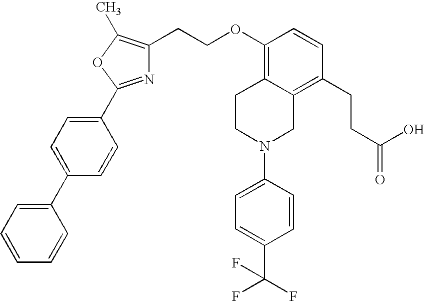

MS (ESI) M/Z (M+H)+ 627.

### Example 115

### 3-[5-[2-(5-Methyl-2-phenyl-oxazol-4-yl)-ethoxy]-2-(5-nitro-2-trifluoromethyl-phenyl)-1,2,3,4-tetrahydro-isoquinolin-8-yl]-propionic acid

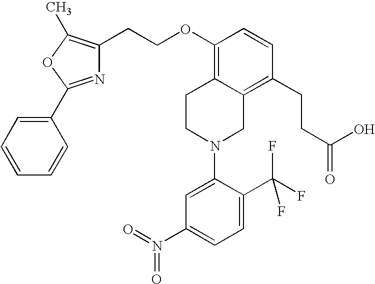

MS (ESI) M/Z (M+H)+ 596.

### Example 116

### 3-{2-(4-Cyano-2-fluoro-phenyl)-5-[2-(5-methyl-2-phenyl-oxazol-4-yl)-ethoxy]-1,2,3,4-tetrahydro-isoquinolin-8-yl}-propionic acid

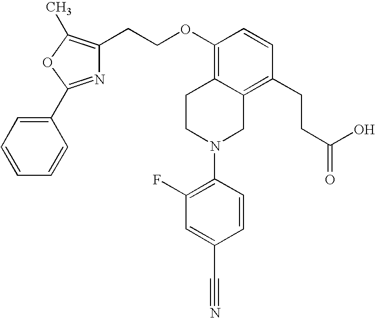

MS (ESI) M/Z (M+H)+ 526.

### Example 117

### 3-{2-(3-Methoxy-phenyl)-5-[2-(5-methyl-2-phenyl-oxazol-4-yl)-ethoxy]-1,2,3,4-tetrahydro-isoquinolin-8-yl}-propionic acid

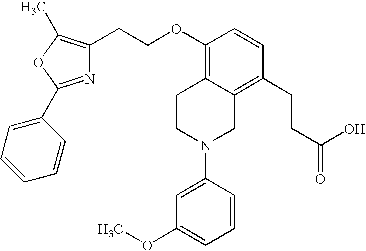

MS (ESI) M/Z (M+H)+ 513.

### Example 118

### 3-{2-(4-tert-Butyl-phenyl)-5-[2-(5-methyl-2-phenyl-oxazol-4-yl)-ethoxy]-1,2,3,4-tetrahydro-isoquinolin-8-yl}-propionic acid

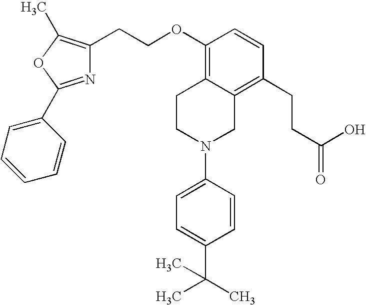

MS (ESI) M/Z (M+H)+ 539.

### Example 119

### 3-{2-Biphenyl-4-yl-5-[2-(5-methyl-2-phenyl-oxazol-4-yl)-ethoxy]-1,2,3,4-tetrahydro-isoquinolin-8-yl}-propionic acid

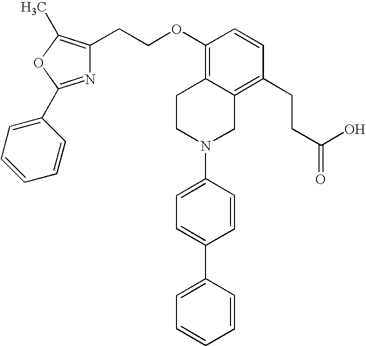

MS (ESI) M/Z (M+H)+ 559.

### Example 120

### ′3-[5-[2-(5-Methyl-2-phenyl-oxazol-4-yl)-ethoxy]-2-(4-nitro-phenyl)-1,2,3,4-tetrahydro-isoquinolin-8-yl]-propionic acid

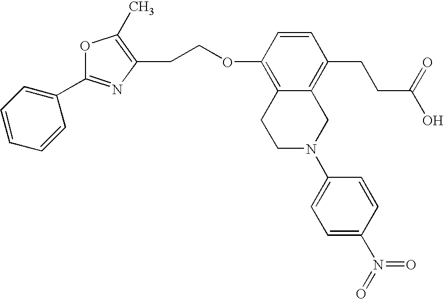

1H NMR (400 MHz, CDCl₃) [symbol] 8.10-8.0 (2H, m), 7.91-7.82 (3H, m), 7.40-7.29 (2H, m), 6.98-6.90 (1H, m), 6.80-6.71 (2H, m), 6.70-6.62 (1H, m), 4.43 (2H, s), 4.19-4.12 (2H, m), 3.6-3.5 (4H, m), 2.95-2.78 (4H, m), 2.62-2.58 (2H, m), 2.30 (3H, s).

### Example 121

### 3-{2-(4-opropyl-phenyl)-5-[2-(5-methyl-2-phenyl-oxazol-4-yl)-ethoxy]-1,2,3,4-tetrahydro-isoquinolin-8-yl}-propionic acid

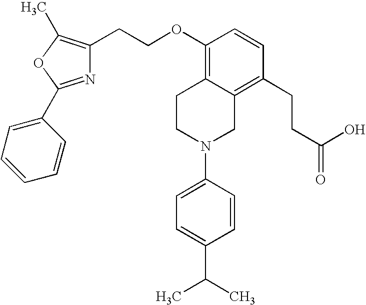

MS (ESI) M/Z (M+H)+ 525.

### Example 122

### 3-{2-(4-Methoxy-phenyl)-5-[2-(5-methyl-2-phenyl-oxazol-4-yl)-ethoxy]-1,2,3,4-tetrahydro-isoquinolin-8-yl}-propionic acid hydrochlorid

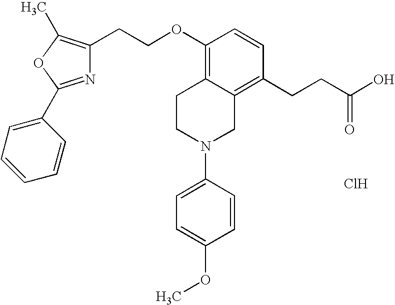

MS (ESI) M/Z (M+H)+ 513.

### Example 123

### 8-(2-Carboxy-ethyl)-5-{1-[4-methyl-2-(4-trifluoromethyl-phenyl)-oxazol-5-yl]-ethoxy}-3,4-dihydro-1H-isoquinoline-2-carboxylic acid tert-butyl ester

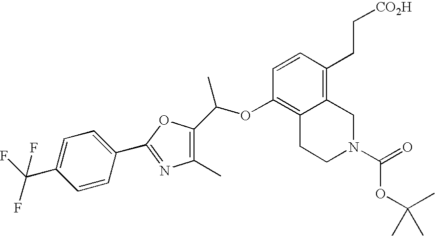

The compound of 1-[4-methyl-2-(4-trifluoromethyl-phenyl)-oxazol-5-yl]-ethanol (0.15 g, 0.55 mmol) is dissolved in 4 mL toluene at 0° C. followed by the addition of tributylphosphine (0.2 mL, 0.83 mmol) and 5-hydroxy-8-(2-methoxycarbonyl-ethyl)-3,4-dihydro-1H-isoquinoline-2-carboxylic acid tert-butyl ester (0.28 g, 0.83 mmol). The compound of 1,1′-(azodicarbonyl)-dipiperidine (0.21 g, 0.883 mmol) is added and after 10 min and the orange solution is warmed to rt and stirred for 24 hrs. The mixture is partitioned between H₂O (25 mL) and ethyl acetate (25 mL). The organic layer is separated and concentrated in vacuo to provide orange oil, which is purified using flash column chromatography (25% acetone/hexane) to afford 0.15 g of the ester (46%). The compound of 8-(2-methoxycarbonyl-ethyl)-5-{1-[4-methyl-2-(4-trifluoromethyl-phenyl)-oxazol-5-yl]-ethoxy}-3,4-dihydro-1H-isoquinoline-2-carboxylic acid tert-butyl ester (0.01 g, 0.017 mmol) is dissolved in 0.5 mL of a solution of THF:MeOH: 1N LiOH (3:2:1). After 2 hrs, saturated ammonium chloride (2 mL) is added, and the aqueous layer is extracted with ethyl acetate. The organics are combined and washed with water (3×2 mL), and the volatiles are evaporated to provide 0.08 g of the title compound (81%). MS (ESI) M/Z (M+H)+ 575.

### Example 124

### 8-(2-Methoxycarbonyl-ethyl)-5-[4-methyl-2-(4-trifluoromethyl-phenyl)-thiazol-5-ylmethoxy]-3,4-dihydro-1H-isoquinoline-2-carboxylic acid tert-butyl ester

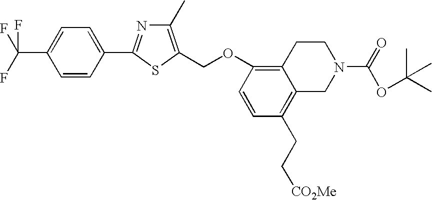

A solution of [4-methyl-2-(4-trifluoromethyl-phenyl)-thiazol-5-yl]-methanol (4.18 g, 15.3 mmol) and triethyl amine (2.66 mL, 19.1 mmol) in methylene chloride (30 mL) is cooled to 0° C., and then MeSO₂Cl (1.48 mL, 19.1 mmol) is added dropwise. The mixture is warmed to rt, stirred for 2 hrs and poured into water (20 mL). The mixture is washed with sodium bicarbonate, water and brine, and then dried over sodium sulfate. Concentration affords the crude chloride, which is used for the next step without further purification. To a acetonitrile (2 mL) solution of 5-chloromethyl-4-methyl-2-(4-trifluoromethyl-phenyl)-thiazole (0.1 g, 0.34 mmol) and 5-hydroxy-8-(2-methoxycarbonyl-ethyl)-3,4-dihydro-1H-isoquinoline-2-carboxylic acid tert-butyl ester (0.13 g, 0.38 mmol) is added Cs₂CO₃ (0.22 g, 0.68 mmol), and the resulting suspension is stirred at rt for 48 hrs. The mixture is poured into water and the layers are separated. The aqueous layer is extracted with methylene chloride (2×15 mL), and the organics are combined and washed with water (3×5 mL). Concentration affords the crude title compound which is purified by flash column chromatography using 10%-25% ethyl acetate/hexane. (0.14 g, 0.25 mmol, 72%). ES/MS+ 591.

Examples 125 to 139 are prepared according to the procedure described in Examples 123 and 124 by using appropriate tail pieces.

### Example 125

### 8-(2-Carboxy-ethyl)-5-[4-methyl-2-(4-trifluoromethyl-phenyl)-oxazol-5-ylmethoxy]-3,4-dihydro-1H-isoquinoline-2-carboxylic acid tert-butyl ester

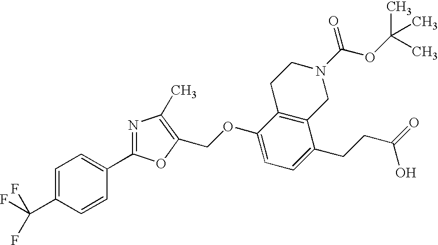

MS (ESI) M/Z (M+H)+ 561.

### Example 126

### 3-{5-[4-Methyl-2-(4-trifluoromethyl-phenyl)-oxazol-5-ylmethoxy]-1,2,3,4-tetrahydro-isoquinolin-8-yl}-propionic acid

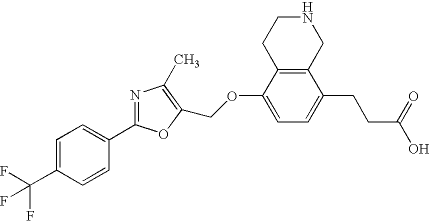

The tert-BOC group is removed as described in Example 1, Step B. MS (ESI) M/Z (M+H)+ 461.

### Example 127

### 8-(2-Carboxy-ethyl)-5-[4-methyl-2-(4-trifluoromethyl-phenyl)-oxazol-5-ylmethoxy]-3,4-dihydro-1H-isoquinoline-2-carboxylic acid propyl ester

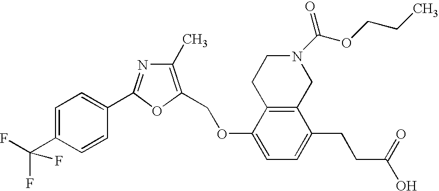

The carbamate is installed as described in Example 1, Steps B to D. ES/MS+ 547.

### Example 128

### 8-(2-Carboxy-ethyl)-5-[4-methyl-2-(4-trifluoromethyl-phenyl)-thiazol-5-ylmethoxy]-3,4-dihydro-1H-isoquinoline-2-carboxylic acid tert-butyl ester

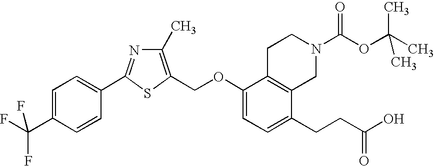

MS (ESI) M/Z (M+H)+ 577.

### Example 129

### 8-(2-Carboxy-ethyl)-5-[4-ethyl-2-(4-trifluoromethyl-phenyl)-thiazol-5-ylmethoxy]-3,4-dihydro-1H-isoquinoline-2-carboxylic acid tert-butyl ester

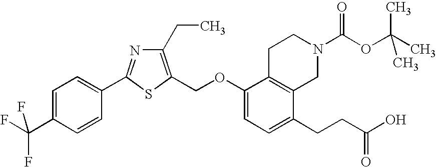

MS (ESI) M/Z (M+H)+ 591.

### Example 130

### 8-(2-Carboxy-ethyl)-5-[5-methyl-2-(4-trifluoromethyl-phenyl)-thiazol-4-ylmethoxy]-3,4-dihydro-1H-isoquinoline-2-carboxylic acid tert-butyl ester

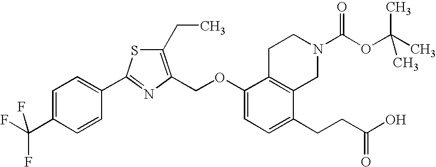

MS (ESI) M/Z (M+H)+ 577.

### Example 131

### 5-[2-(4-Bromo-phenyl)-5-methyl-oxazol-4-ylmethoxy]-8-(2-carboxy-ethyl)-3,4-dihydro-1H-isoquinoline-2-carboxylic acid tert-butyl ester

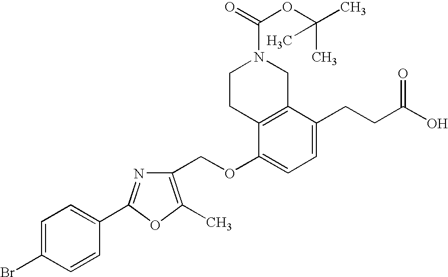

MS (ESI) M/Z (M+H)+ 572.

### Example 132

### 3-{2-Acetyl-5-[4-methyl-2-(4-trifluoromethyl-phenyl)-oxazol-5-ylmethoxy]-1,2,3,4-tetrahydro-isoquinolin-8-yl}-propionic acid

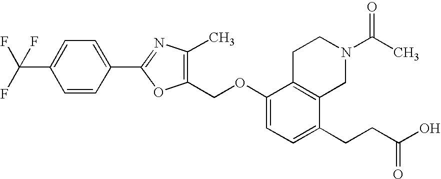

The acetamide is installed as described in Example 1, Steps B to D.

MS (ESI) M/Z (M+H)+ 503.

### Example 133

### 8-(2-Carboxy-ethyl)-5-[4-methyl-2-(4-trifluoromethyl-phenyl)-oxazol-5-ylmethoxy]-3,4-dihydro-1H-isoquinoline-2-carboxylic acid methyl ester

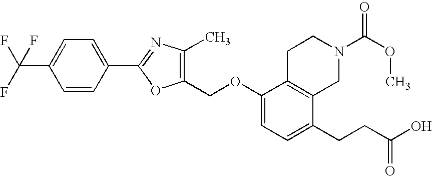

The carbamate is installed as described in Example 1, Steps B to D.

MS (ESI) M/Z (M+H)+ 519

### Example 134

### 3-{2-Methanesulfonyl-5-[4-methyl-2-(4-trifluoromethyl-phenyl)-oxazol-5-ylmethoxy]-1,2,3,4-tetrahydro-isoquinolin-8-yl}-propionic acid

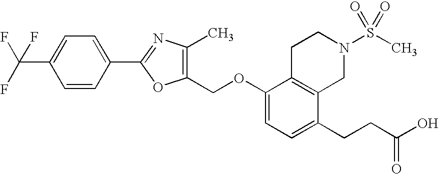

The sulfonamide is installed as described in Example 1, Steps B to D.

MS (ESI) M/Z (M+H)+ 539.

### Example 135

### 8-(2-Carboxy-ethyl)-5-(5-methyl-2-phenyl-oxazol-4-ylmethoxy)-3,4-dihydro-1H-isoquinoline-2-carboxylic acid tert-butyl ester

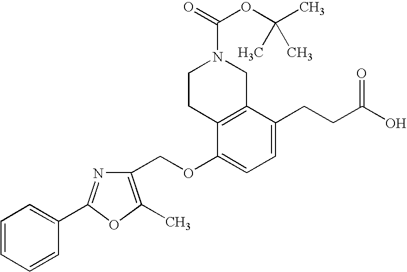

MS (ESI) M/Z (M+H)+ 493.

### Example 136

### 8-(2-Carboxy-ethyl)-5-[2-(4-fluoro-phenyl)-5-methyl-oxazol-4-ylmethoxy]-3,4-dihydro-1H-isoquinoline-2-carboxylic acid tert-butyl ester

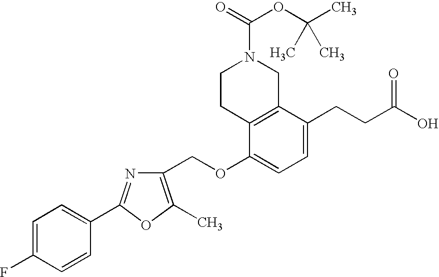

MS (ESI) M/Z (M+H)+ 511.

### Example 137

### 8-(2-Carboxy-ethyl)-5-{2-[5-ethyl-2-(4-trifluoromethyl-phenyl)-thiazol-4-yl]-ethoxy}-3,4-dihydro-1H-isoquinoline-2-carboxylic acid tert-butyl ester

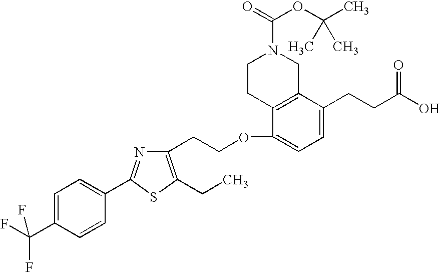

MS (ESI) M/Z (M+H)+ 605.

### Example 138

### 5-[2-(4-Bromo-phenyl)-4-methyl-thiazol-5-ylmethoxy]-8-(2-carboxy-ethyl)-3,4-dihydro-1H-isoquinoline-2-carboxylic acid tert-butyl ester

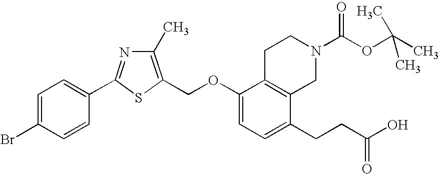

MS (ESI) M/Z (M+H)+ 588.

### Example 139

### 3-{2-Methylcarbamoyl-5-[4-methyl-2-(4-trifluoromethyl-phenyl)-oxazol-5-ylmethoxy]-1,2,3,4-tetrahydro-isoquinolin-8-yl}-propionic acid

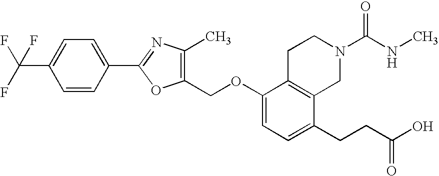

The urea is installed as described in Example 1, Steps B to D. MS (ESI) M/Z (M+H)+ 518.

### Example 140

### 4-(2-Carboxyethyl)-7-[2-(5-methyl-2-phenyloxazol-4-yl)ethoxy]-1,3-dihydroisoindole-2-carboxylic acid t-butyl ester

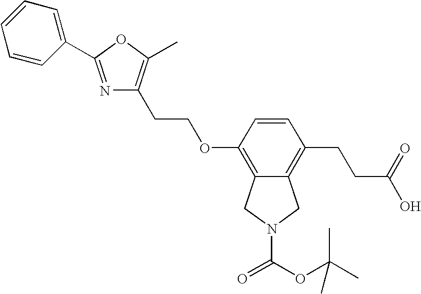

### Step A

### 2-Benzyl-4-bromo-7-methoxy-2,3-dihydro-1H-isoindole

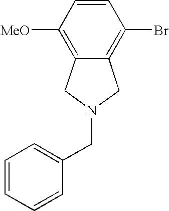

To a 50 mL round bottom flask are charged with 1-bromo-2,3-bromomethyl-4-methoxybenzene (0.50 g, 1.34 mmol) [JOC, 57, 6374-6376, (1992)] and anhydrous THF (20 mL), followed by benzylamine (0.146 mL, 1.34 mmol), sodium carbonate (0.284 g 2.68 mmol), and tetrabutylammonium iodide (0.10 g 0.26 mmol). The mixture is heated at reflux for 20 h. After cooling, the mixture is diluted with EtOAc, washed with brine, dried and concentrated. The crude residue is purified using radial chromatography (4 mm silica gel plate, 5:95 to 10:90 EtOAc:hex) to give a brown oil (0.20 g, 47%). ¹H NMR (400 MHz, CDCl₃) δ 3.77 (s, 3H), 3.92 (s, 2H), 3.98 (s, 2H), 4.01 (s, 2H), 6.58 (d, J=8.8 Hz, 1H), 7.25-7.42 (m, 6H). MS (ES) m/e 318 (M+1).

### Step B

### 4-Bromo-7-methoxy-2,3-dihydro-1H-isoindole hydrochloride

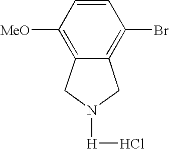

To a 100 mL round bottom flask is charged with 2-benzyl-4-bromo-7-methoxy-2,3-dihydro-1H-isoindole (1.93 g, 6.06 mmol) in anhydrous MeCl₂ (40 mL). To the mixture, 1-chloroethyl chloroformate (3.3 ml, 30.3 mmol) is added, and the solution is stirred at ambient temperature for 16 h. The reaction is diluted with methanol (40 mL), heated at reflux for 2 h, and concentrated to give a green solid (0.98 g, 65%), which is used directly in the next step. ¹H NMR (400 MHz, CDCl₃) δ 3.79 (d, J=0.8 Hz, 3H), 4.44 (d, J=19.9 Hz, 4H), 6.96 (d, J=8.6 Hz, 1H), 7.51 (d, J=9.0 Hz, 1H), 10.1 (s, 1H), MS (Exact Mass) Calc: 228.0024; Found: 228.0022.

### Step C

### 1-(4-Bromo-7-methoxy-1,3-dihydroisoindol-2-yl)-2,2,2-trifluoroethanone

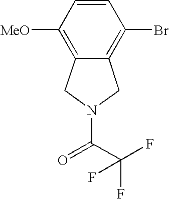

To a flame dried 100 mL round bottom flask under a nitrogen atmosphere are charged with 4-bromo-7-methoxy-2,3-dihydro-1H-isoindole hydrochloride salt (0.97 g 3.87 mmol) and anhydrous MeCl₂ (20 mL), followed by triethylamine (1.08 mL, 7.74 mmol) and 4-dimethylaminopyridine (0.10 g). To the mixture, trifluoroacetic anhydride (0.55 mL, 3.87 mmol) is added slowly. The mixture is stirred for 16 h and poured into 150 mL of EtOAc. The organic layer is washed with brine, saturated NaHCO₃, and brine, which is then dried and concentrated. The crude solid is purified using radial chromatography (4 mm silica gel plate, 2:98 to 5:95 EtOAc:hex) to give a yellow solid (0.84 g, 67%). ¹H NMR (400 MHz, CDCl₃) δ 3.85 (dd, J=6.6, 2.7 Hz, 3H), 4.87 (s, 1H), 4.94 (d, J=14.9 Hz, 2H), 5.03 (s, 1H), 6.72-6.74 (m, 1H), 7.41-7.44 (m, 1H), MS (ES) m/e 325 (M+1).

### Step D

### 1-(4-Bromo-7-hydroxy-1,3-dihydroisoindol-2-yl)-2,2,2-trifluoroethanone

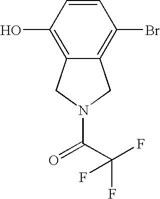

To a 1 L round bottom flask under a nitrogen atmosphere is charged with 1-(4-bromo-7-methoxy-1,3-dihydroisoindol-2-yl)-2,2,2-trifluoroethanone (18.3 g, 56.5 nmol) and anhydrous MeCl₂ (180 mL). The solution is cooled in an ice/alcohol bath and treated dropwise with boron tribromide (169 mL, 169 mmol). The reaction is stirred for 2 h and diluted with 1.2 L EtOAc and ice water. The mixture is washed 400 mL water and brine, and then dried and concentrated to give a yellow solid (17.0 g, 97%). ¹H NMR (400 MHz, CDCl₃) δ 1.61 (br s. 1H), 4.86 (s, 1H), 4.97 (d, J=4.69, 2H), 5.07 (s, 1H), 6.67 (dd, J=12.3, 8.4 Hz, 1H), 7.31 (d, 8.6 Hz, 1H), MS (ES) m/e 311 (M+1).

### Step E

### 4-Bromo-7-hydroxy-1,3-dihydroisoindole-2-carboxylic acid t-butyl ester

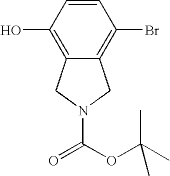

To a 1 L round bottom flask is charged with 1-(4-bromo-7-hydroxy-1,3-dihydroisoindol-2-yl)-2,2,2-trifluoroethanone (16.9 g, 54.5 mmol), methanol (500 mL), and K₂CO₃ (7.53 g, 54.5 mmol). The reaction is heated at 50° C. for 72 h, and then filtered and concentrated. The residue is dissolved in acetone and aqueous Na₂CO₃ solution (90 mL each). The reaction is cooled in an ice bath and treated with di-tert-butyl dicarbonate (11.9 g, 54.5 mmol). The ice bath is removed, and the reaction is stirred at ambient temperature for 20 h. The mixture is diluted with IL EtOAc and washed with 500 mL water and brine. The organic layer is dried and concentrated. The residue is triturated with ether:hex (1:1; 220 mL), filtered, and dried to give a gray solid (12.0 g, 70% over two steps). ¹H NMR (400 MHz, DMSO-d₆) δ 1.41 (s, 9H), 4.42-4.52 (m, 4H), 6.64 (d, J=8.6 Hz, 1H), 7.23 (d, J=8.6 Hz, 1H), 9.94 (d, J=7.0 Hz, 1H), MS (ES) m/e 313 (M−1).

### Step F

### 4-Hydroxy-7-(2-methoxycarbonylvinyl)-1,3-dihydroisoindole-2-carboxylic acid t-butyl ester

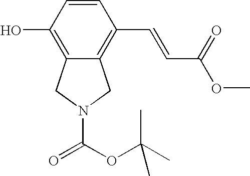

To a 500 mL round bottom flask under a nitrogen atmosphere are charged with 4-bromo-7-hydroxy-1,3-dihydroisoindole-2-carboxylic acid t-butyl ester (6.90 g, 21.96 mmol), propionitrile (135 mL), tri-o-tolylphosphine (1.67 g, 5.49 mmol), and diisopropylamine (7.64 mL, 43.9 mmol). The flask is degassed and flushed with nitrogen, and methyl acrylate (5.93 mL, 65.9 mmol) is added. The flask is degassed and flushed with nitrogen, and palladium acetate (0.62 g, 2.75 mmol) is added. The reaction is heated at 97° C. for 20 h. After cooling, the gray mixture is treated with 130 mL ether. The precipitate is filtered, washed with ether:hex (1:1; 75 mL), and dried to give the title compound as a tan solid (4.25 g, 55%). ¹H NMR (400 MHz, CDCl₃) δ 1.43 (s, 9H), 3.67 (s, 3H), 4.42-4.51 (m, 2H), 4.66-4.68 (m, 2H), 6.23 (t, J=14.8 Hz, 1H), 6.72 (d, J=8.6 Hz, 1H), 7.44-7.55 (m, 2H), 10.3 (d, J=10.6 Hz, 1H), (ES) m/e 319 (M−1).

### Step G

### 4-Hydroxy-7-(2-methoxycarbonylethyl)-1,3-dihydroisoindole-2-carboxylic acid t-butyl ester

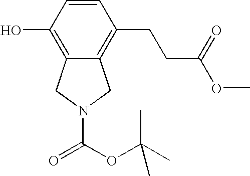

To a Parr bottle are charged with 4-hydroxy-7-(2-methoxycarbonylvinyl)-1,3-dihydroisoindole-2-carboxylic acid t-butyl ester (2.24 g, 7.39 mmol), THF, MeOH (50 mL each), and 5% Pd/C (0.28 g). The mixture is hydrogenated at 60 psi at ambient temperature for 3 h. The catalyst is filtered, and the filtrate is concentrated to give a white solid (2.0 g, 89%). ¹H NMR (400 MHz, DMSO-d₆) δ 1.44 (s, 9H), 2.54 (t, J=5.9 Hz, 2H), 2.63 (t, J=7.0 Hz, 2H), 3.55 (s, 3H), 4.43 (d, J=10.9 Hz, 2H), 4.53 (s, 2H), 6.61 (d, J=7.8 Hz, 1H), 6.89 (d, J=8.2 Hz, 1H), 9.45 (br s, 1H), (ES) m/e 322 (M+1).

### Step H

### 4-(2-Methoxycarbonylethyl)-7-[2-(5-methyl-2-phenyloxazol-4-yl)ethoxy]-1,3-dihydroisoindole-2-carboxylic acid t-butyl ester

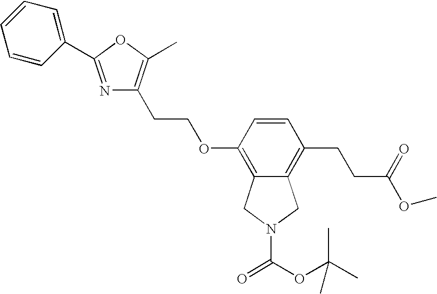

To a 50 mL round bottom flask under nitrogen are charged with 4-hydroxy-7-(2-methoxycarbonylethyl)-1,3-dihydroisoindole-2-carboxylic acid t-butyl ester (0.92 g, 3.01 mmol) and toluene-4-sulfonic acid 2-(5-methyl-2-phenyloxazol-4-yl)ethyl ester (1.40 g, 3.92 mmol), and anhydrous DMF (10 mL). To the mixture, cesium carbonate (3.2 g, 9.83 mmol) is added. The reaction is heated to 55° C. for 16 h and concentrated. The residue is diluted with 300 mL EtOAc and washed with water (500 mL) and brine (500 mL), and then dried and concentrated. The residue is purified by medium pressure silica gel chromatography (5:95 to 10:90 EtOAc:MeCl₂) to give a white solid (0.97 g, 64%). ¹H NMR (400 MHz, CDCl₃) δ 1.52 (d, J=1.6 Hz, 9H), 2.39 (d, J=8.6 Hz, 3H), 2.56 (quintet, J=5.7 Hz, 2H), 2.80 (t, J=7.8 Hz, 2H), 2.97 (q, J=5.9 Hz, 2H), 3.66 (d, J=7.8 Hz, 3H), 4.24 (quintet, 2.6 Hz, 2H), 4.54 (s, 1H), 4.59 (s, 2H), 4.66 (s, 1H), 6.71 (d, J=8.2 Hz, 1H), 7.00 (dd, J=8.4, 4.9 Hz, 1H), 7.40-7.45 (m, 4H), 7.98 (dd, J=7.4, 2.0 Hz, 2H), (ES) m/e 507 (M+1).

### Step I

### 4-(2-Carboxyethyl)-7-[2-(5-methyl-2-phenyloxazol-4-yl)ethoxy]-1,3-dihydroisoindole-2-carboxylic acid t-butyl ester

To a 25 mL round bottom flask is charged with 4-(2-methoxycarbonylethyl)-7-[2-(5-methyl2-phenyloxazol-4-yl)ethoxy]-1,3-dihydroisoindole-2-carboxylic acid t-butyl ester (0.023 g, 0.045 mmol), MeOH (1 mL), and 2N NaOH (0.2 mL). The stirred solution is heated to 50° C. for 2 h. The volatiles are concentrated, and the residue is treated with 3 mL ice water and acidified to pH 1 using 2N HCl. The mixture is extracted with 20 mL EtOAc and washed with water, and then dried and concentrated to give a white solid (0.021 g, 95%). ¹H NMR (400 MHz, CDCl₃) δ 1.52 (s, 9H), 2.39 (d, J=8.6 Hz, 3H), 2.53-2.60 (m, 2H), 2.80 (t, J=7.8 Hz, 2H), 4.24 (d, J=2.7 Hz, 2H), 4.54 (s, 1H), 4.59 (s, 2H), 4.66 (s, 1H), 6.71 (d, J=8.2 Hz, 1H), 7.00 (t, J=6.3 Hz, 1H), 7.42 (d, J=5.5 Hz, 3H), 7.98 (d, J=6.3 Hz, 2H), (ES) m/e 493 (M+1).

### Example 141

### 3-{2-(4-Butoxy-benzenesulfonyl)-7-[2-(5-methyl-2-phenyloxazol-4-yl)ethoxy]-2,3-dihydro-1H-isoindol-4-yl}propionic acid

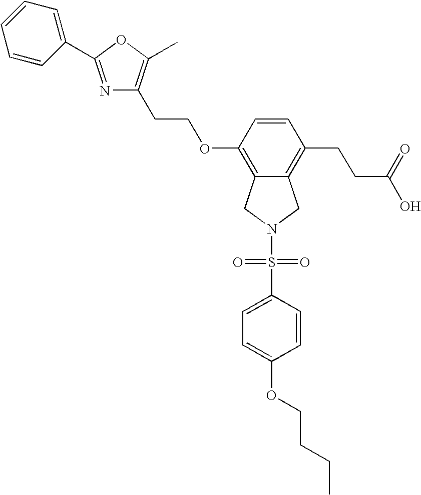

The title compound is prepared according to the scheme shown below:

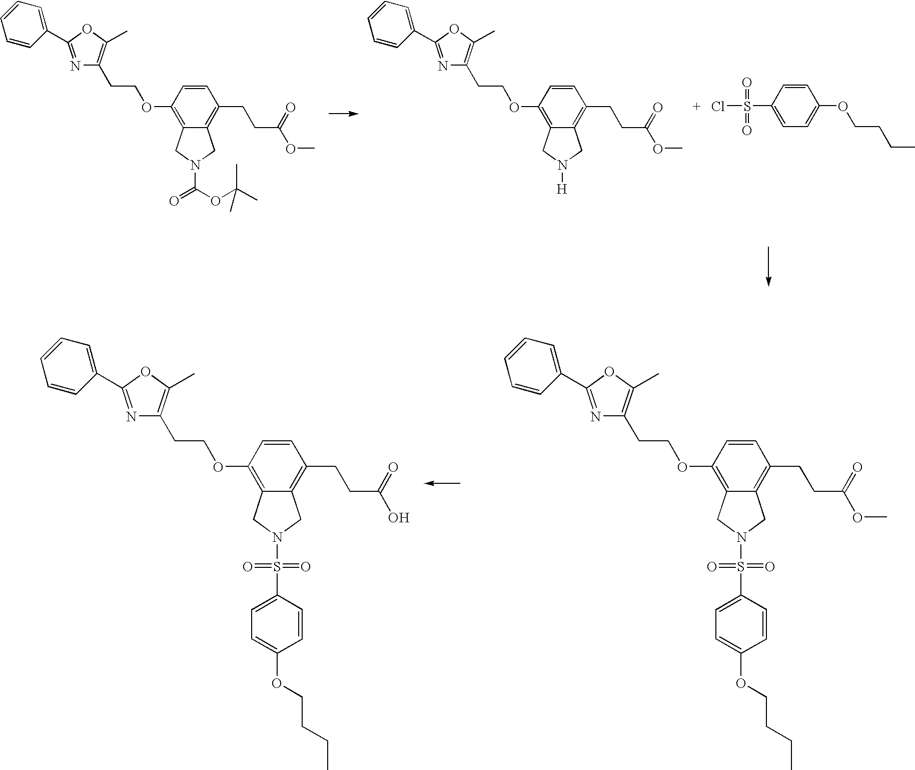

### Step A

### 3-{7-[2-(5-Methyl-2-phenyloxazol-4-yl)ethoxy]-2,3-dihydro-1H-isoindol-4-yl}propionic acid methyl ester

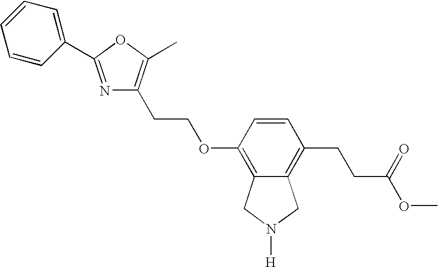

A 100 mL round bottom flask is charged with 4-(2-methoxy-carbonylethyl)-7-[2-(5-methyl-2-phenyloxazol-4-yl)ethoxy]-1,3-dihydroisoindole-2-carboxylic acid t-butyl ester (0.95 g, 1.88 mmol) and MeCl₂ (16 mL) followed by addition of TFA (8 mL). The solution is stirred at ambient temperature for 2 h and concentrated. The residue is dissolved in 50 mL of EtOAc and washed with saturated NaHCO₃, and then dried and concentrated to give a tan solid (0.70 g, 92%). ¹H NMR (400 MHz, CDCl₃) δ 2.37 (s, 3H), 2.55 (t, J=7.8 Hz, 2H), 2.82 (t, J=7.6 Hz, 2H), 2.96 (t, J=6.6 Hz, 2H), 3.66 (s, 3H), 4.24 (t, J=6.6 Hz, 2H), 6.69 (d, J=8.2 Hz, 1H), 6.98 (d, J=8.2 Hz, 1H) 7.37-7.45 (m, 3H), 7.95-7.99 (m, 2H), (ES) m/e 407 (M+1).

### Step B

### 3-{2-(4-Butoxy-benzenesulfonyl)-7-[2-(5-methyl-2-phenyloxazol-4-yl)ethoxy]-2,3-dihydro-1H-isoindol-4-yl}propionic acid

General procedure for the parallel synthesis of analogs utilizing the DynaVac carousel. A 50 mL glass tube with screw cap and nitrogen inlet charged with 3-{7-[2-(5-methyl-2-phenyloxazol-4-yl)ethoxy]-2,3-dihydro-1H-isoindol-4-yl}propionic acid methyl ester (0.030 g, 0.084 mmol) is dissolved in 1 mL of anhydrous MeCl₂. To the mixture, TEA (0.024 mL, 0.17 mmol) and 4-butoxy-benzenesulfonyl chloride (0.022 g, 0.17 mmol) are added. The mixture is stirred at ambient temperature for 16 h, and then concentrated under a stream of nitrogen. The crude 3-{2-(4-butoxy-benzenesulfonyl)-7-[2-(5-methyl-2-phenyloxazol-4-yl)ethoxy]-2,3-dihydro-1H-isoindol-4-yl}propionic acid methyl ester is treated with 1 mL of MeOH and 2N NaOH (0.21 mL). The mixture is heated at 55° C. for 3 h and concentrated under a stream of nitrogen. The residue is treated with MeCl₂ (1 mL), water (0.5 mL) and 5N HCl (0.19 mL). The mixture is poured into a Varian Chem Elut 1003 cartridge and eluted with MeCl₂. The crude product is purified by mass-directed reverse phase HPLC to provide 0.022 g (51%) of the title compound. MS (ES) m/e 605 (M+1).

### Examples 142 to 162 are prepared according to the procedure described in Example 141

### Example 142

### 3-{2-(2,5-Dichlorothiophene-3-carbonyl)-7-[2-(5-methyl-2-phenyloxazol-4-yl)ethoxy]-2,3-dihydro-1H-isoindol-4-yl}propionic acid

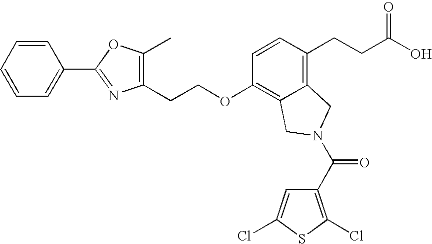

MS (ES) m/e 572 (M+1).

### Example 143

### 3-{2-Benzoyl-7-[2-(5-methyl-2-phenyloxazol-4-yl)ethoxy]-2,3-dihydro-1H-isoindol-4-yl}propionic acid

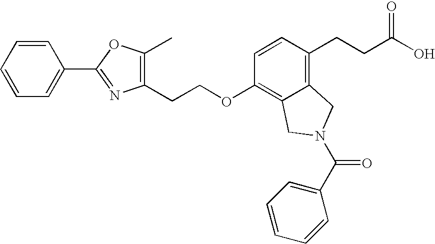

MS (ES) m/e 497 (M+1).

### Example 144

### 3-[7-[2-(5-Methyl-2-phenyloxazol-4-yl)ethoxy]-2-(2-phenoxyacetyl)-2,3-dihydro-1H-isoindol-4-yl]propionic acid

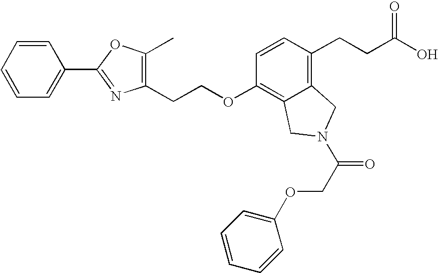

MS (ES) m/e 527 (M+1).

### Example 145

### 3-{2-(Biphenyl-4-sulfonyl)-7-[2-(5-methyl-2-phenyloxazol-4-yl)ethoxy]-2,3-dihydro-1H-isoindol-4-yl}propionic acid

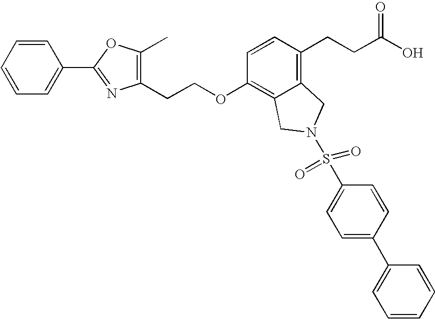

MS (ES) m/e 609 (M+1).

### Example 146

### 4-(2-Carboxyethyl)-7-[2-(5-methyl-2-phenyloxazol-4-yl)ethoxy]-1,3-dihydroisoindole-2-carboxylic acid isobutylester

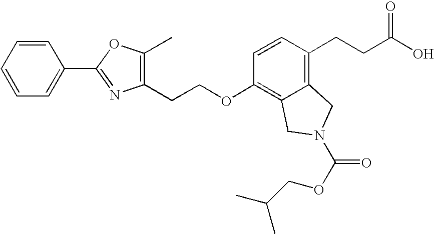

MS (ES) m/e 493 (M+1).

### Example 147

### 4-(2-Carboxyethyl)-7-[2-(5-methyl-2-phenyloxazol-4-yl)ethoxy]-1,3-dihydroisoindole-2-carboxylic acid opropyl ester

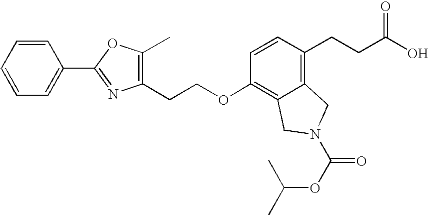

MS (ES) m/e 479 (M+1).

### Example 148

### 3-[7-[2-(5-Methyl-2-phenyloxazol-4-yl)ethoxy]-2-(3-phenylpropionyl)-2,3-dihydro-1H-isoindol-4-yl]propionic acid

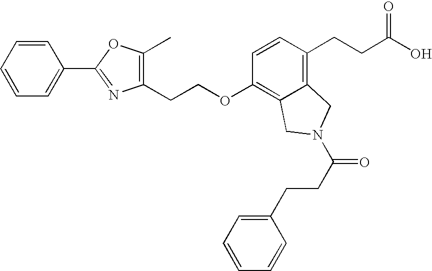

MS (ES) m/e 525 (M+1).

### Example 149

### 3-{2-Butyryl-7-[2-(5-methyl-2-phenyloxazol-4-yl)ethoxy]-2,3-dihydro-1H-isoindol-4-yl}propionic acid

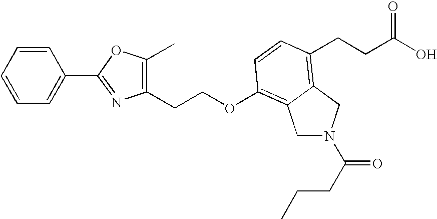

MS (ES) m/e 463 (M+1).

### Example 150

### 4-(2-Carboxyethyl)-7-[2-(5-methyl-2-phenyloxazol-4-yl)ethoxy]-1,3-dihydroisoindole-2-carboxylic acid propyl ester

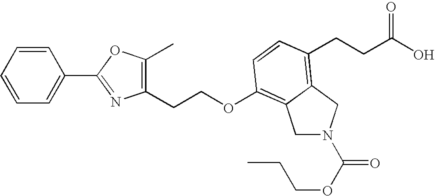

MS (ES) m/e 479 (M+1).

### Example 151

### 3-{2-(3,5-B-trifluoromethylbenzoyl)-7-[2-(5-methyl-2-phenyloxazol-4-yl)ethoxy]-2,3-dihydro-1H-isoindol-4-yl}propionic acid

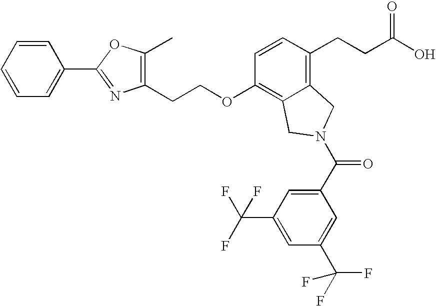

MS (ES) m/e 633 (M+1).

### Example 152

### 4-(2-Carboxyethyl)-7-[2-(5-methyl-2-phenyloxazol-4-yl)ethoxy]-1,3-dihydroisoindole-2-carboxylic acid benzyl ester

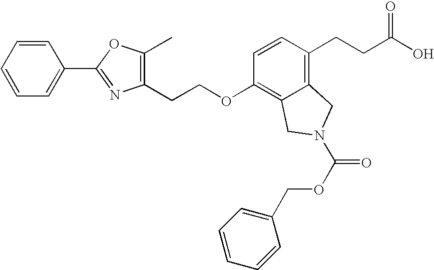

MS (ES) m/e 633 (M+1).

### Example 153

### 3-[7-[2-(5-Methyl-2-phenyloxazol-4-yl)ethoxy]-2-(5-pyridin-2-yl-thiophene-2-sulfonyl)-2,3-dihydro-1H-isoindol-4-yl]propionic acid

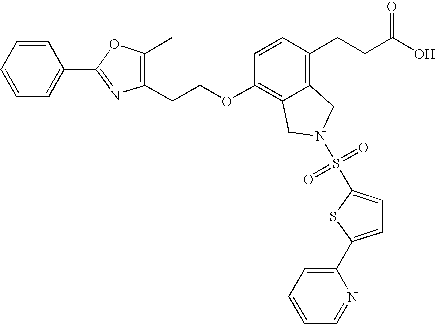

MS (ES) m/e 616 (M+1).

### Example 154

### 3-{2-Benzenesulfonyl-7-[2-(5-methyl-2-phenyloxazol-4-yl)ethoxy]-2,3-dihydro-1H-isoindol-4-yl}propionic acid

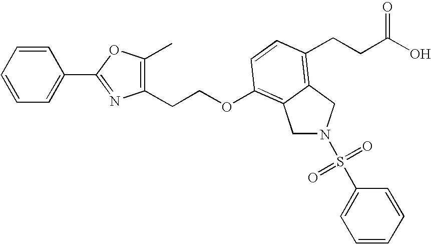

MS (ES) m/e 533 (M+1).

### Example 155

### 3-[7-[2-(5-Methyl-2-phenyloxazol-4-yl)ethoxy]-2-(4-trifluoromethylbenzenesulfonyl)-2,3-dihydro-1H-isoindol-4-yl]propionic acid

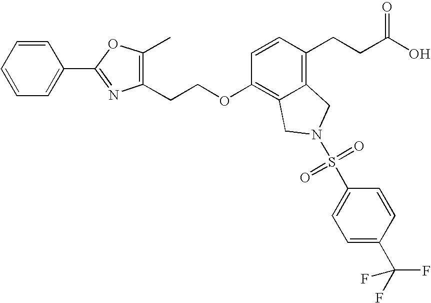

MS (ES) m/e 601 (M+1).

### Example 156

### 4-(2-Carboxyethyl)-7-[2-(5-methyl-2-phenyloxazol-4-yl)ethoxy]-1,3-dihydroisoindole-2-carboxylic acid 2-methoxyethyl ester

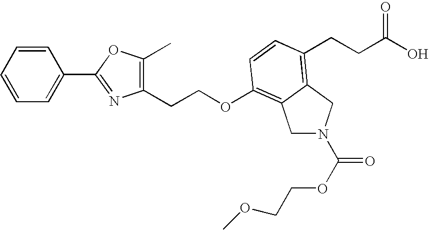

MS (ES) m/e 495 (M+1).

### Example 157

### 3-{2-Benzylcarbamoyl-7-[2-(5-methyl-2-phenyloxazol-4-yl)ethoxy]-2,3-dihydro-1H-isoindol-4-yl}propionic acid

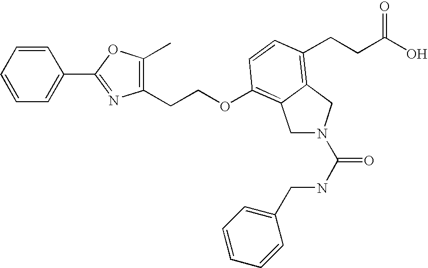

MS (ES) m/e 526 (M+1).

### Example 158

### 3-[7-[2-(5-Methyl-2-phenyloxazol-4-yl)ethoxy]-2-(pyridine-3-carbonyl)-2,3-dihydro-1H-isoindol-4-yl]propionic acid

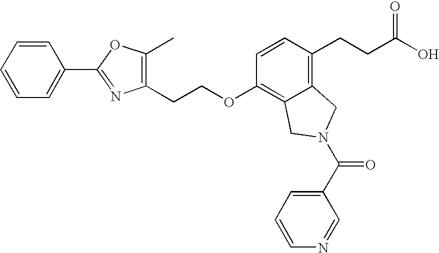

MS (ES) m/e 498 (M+1).

### Example 159

### 3-{2-(5-Methylhexanoyl)-7-[2-(5-methyl-2-phenyloxazol-4-yl)ethoxy]-2,3-dihydro-1H-isoindol-4-yl}propionic acid

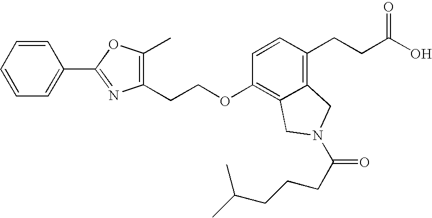

MS (ES) m/e 505 (M+1).

### Example 160

### 3-[7-[2-(5-Methyl-2-phenyloxazol-4-yl)ethoxy]-2-(2-thiophen-2-yl-acetyl)-2,3-dihydro-1H-isoindol-4-yl]propionic acid

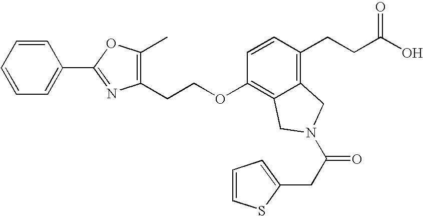

MS (ES) m/e 517 (M+1).

### Example 161

### 4-(2-Carboxyethyl)-7-[2-(5-methyl-2-phenyloxazol-4-yl)ethoxy]-1,3-dihydroisoindole-2-carboxylic acid 2,24-dimethylpropyl ester

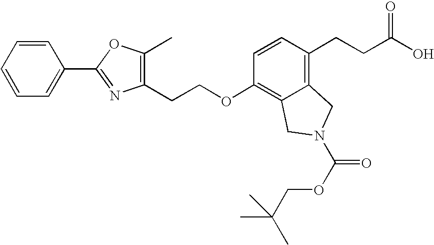

MS (ES) m/e 507 (M+1).

### Example 162

### 3-[7-[2-(5-Methyl-2-phenyloxazol-4-yl)ethoxy]-2-(thiophene-2-carbonyl)-2,3-dihydro-1H-isoindol-4-yl]propionic acid

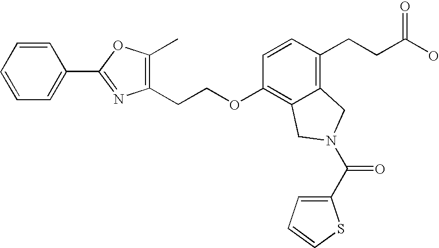

MS (ES) m/e 503 (M+1).

### Example 163

### 3-{2-(2-Cyclohexylacetyl)-7-[2-(5-methyl-2-phenyloxazol-4-yl)ethoxy]-2,3-dihydro-1H-isoindol-4-yl}propionic acid

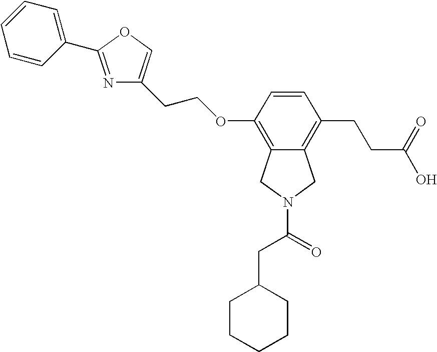

The title compound is prepared according to the scheme shown below:

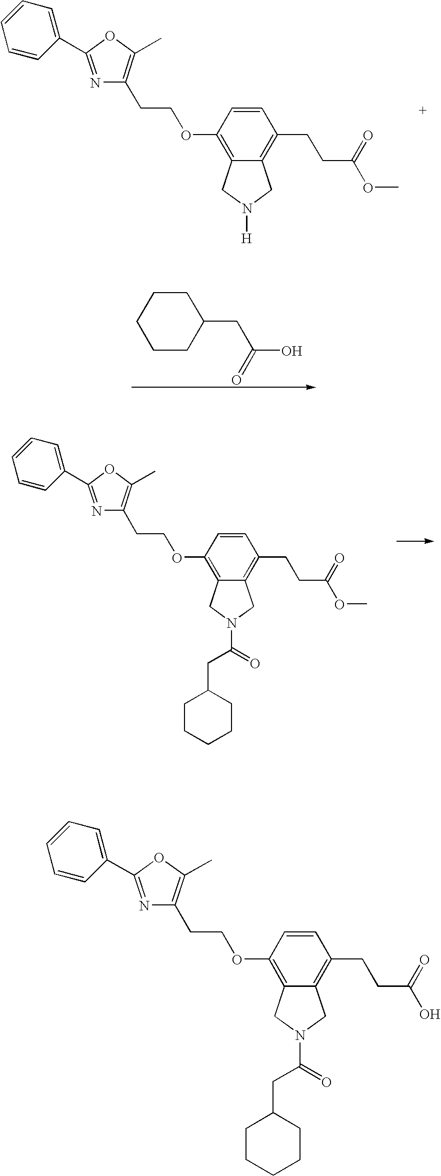

General procedure for the parallel synthesis of analogs utilizing the DynaVac carousel. To a 50 mL glass tube with screw cap and nitrogen inlet are charged with 3-{7-[2-(5-methyl-2-phenyloxazol-4-yl)ethoxy]-2,3-dihydro-1H-isoindol-4-yl}propionic acid methyl ester (0.030 g, 0.084 mmol), anhydrous MeCl₂ (1 mL), 1-hydroxybenzotriazole hydrate (0.017 g, 0.13 mmol), 4-dimethylaminopyridine (0.010 g), and 1-[3-(dimethylamino)propyl]-3-ethylcarbodiimide hydrochloride chloride. The mixture is stirred at ambient temperature for 16 h. The mixture is concentrated using a stream of nitrogen. The crude 3-{2-(2-cyclohexyl-acetyl)-7-[2-(5-methyl-2-phenyloxazol-4-yl)ethoxy]-2,3-dihydro-1H-isoindol-4-yl}propionic acid methyl ester is dissolved in 1 mL of MeOH, treated with 2N NaOH (0.21 mL), heated at 55° C. for 3 h, and concentrated using a stream of nitrogen. The residue is treated with MeCl₂ (1 mL), water (0.5 mL), and 5N HCl (0.19 mL). The mixture is poured into a Varian Chem Elut 1003 cartridge and eluted with MeCl₂. The crude residue is purified by mass-directed reverse phase HPLC to provide 0.016 g (37%) of the title compound. MS (ES) m/e 517 (M+1).

Examples 164 to 172 are prepared according to the procedure described in Example 163.

### Example 164

### 3-{2-Diphenylacetyl-7-[2-(5-methyl-2-phenyloxazol-4-yl)ethoxy]-2,3-dihydro-1H-isoindol-4-yl}propionic acid

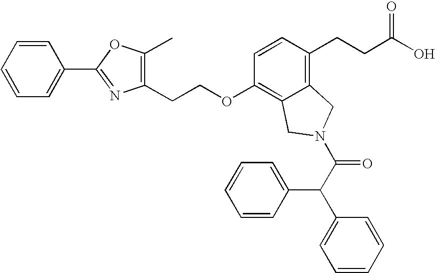

MS (ES) m/e 587 (M+1).

### Example 165

### 3-[7-[2-(5-Methyl-2-phenyloxazol-4-yl)-ethoxy]-2-(pyrazine-2-carbonyl)-2,3-dihydro-1H-isoindol-4-yl]propionic acid

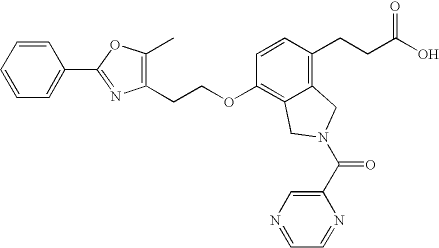

MS (ES) m/e 499 (M+1).

### Example 166

### 3-{2-(5-Chlorothiophene-2-carbonyl)-7-[2-(5-methyl-2-phenyloxazol-4-yl)ethoxy]-2,3-dihydro-1H-isoindol-4-yl}propionic acid

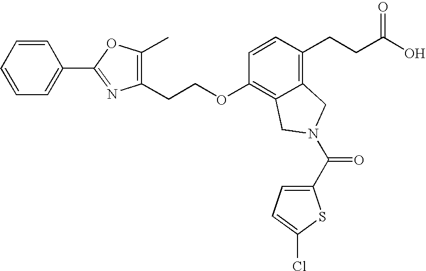

MS (ES) m/e 538 (M+1).

### Example 167

### 3-[7-[2-(5-Methyl-2-phenyloxazol-4-yl)ethoxy]-2-(pyridine-2-carbonyl)-2,3-dihydro-1H-isoindol-4-yl]propionic acid

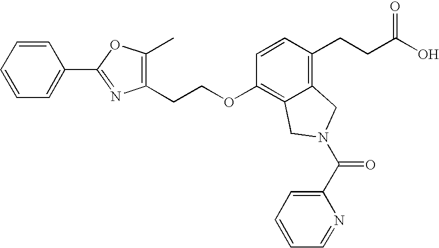

MS (ES) m/e 498 (M+1).

### Example 168

### 3-{2-(3-Chlorothiophene-2-carbonyl)-7-[2-(5-methyl-2-phenyloxazol-4-yl)ethoxy]-2,3-dihydro-1H-isoindol-4-yl}propionic acid

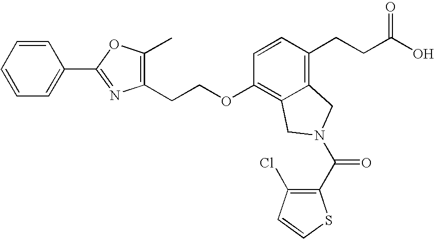

MS (ES) m/e 538 (M+1).

### Example 169

### 3-[7-[2-(5-Methyl-2-phenyloxazol-4-yl)ethoxy]-2-(5-methylthiophene-2-carbonyl)-2,3-dihydro-1H-isoindol-4-yl]propionic acid

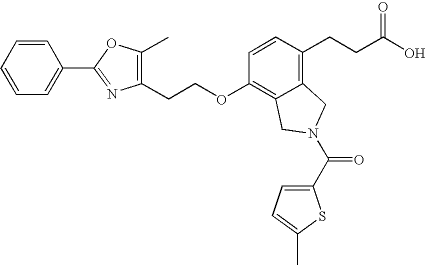

MS (ES) m/e 517 (M+1).

### Example 170

### 3-[7-[2-(5-Methyl-2-phenyloxazol-4-yl)ethoxy]-2-(thiophene-3-carbonyl)-2,3-dihydro-1H-isoindol-4-yl]propionic acid

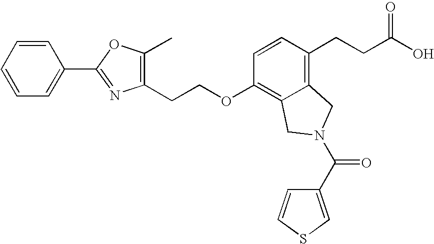

MS (ES) m/e 503 (M+1).

### Example 171

### 3-{2-(3-Ethoxythiophene-2-carbonyl)-7-[2-(5-methyl-2-phenyloxazol-4-yl)ethoxy]-2,3-dihydro-1H-isoindol-4-yl}propionic acid

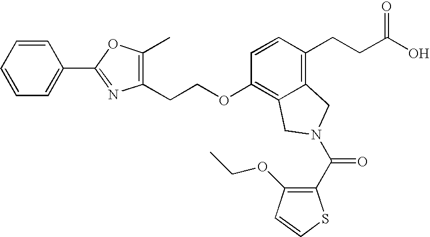

MS (ES) m/e 547 (M+1).

### Example 172

### 3-{2-(4-Methoxythiophene-3-carbonyl)-7-[2-(5-methyl-2-phenyloxazol-4-yl)ethoxy]-2,3-dihydro-1H-isoindol-4-yl}propionic acid

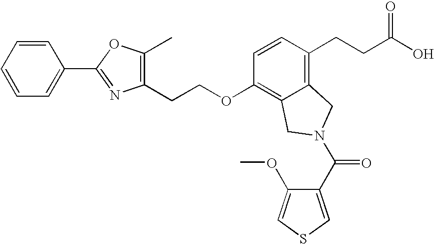

MS (ES) m/e 533 (M+1).

### Example 173

### 3-[7-[2-(2-Biphenyl-4-yl-5-methyloxazol-4-yl)ethoxy]-2-(butane-1-sulfonyl)-2,3-dihydro-1H-isoindol-4-yl]propionic acid

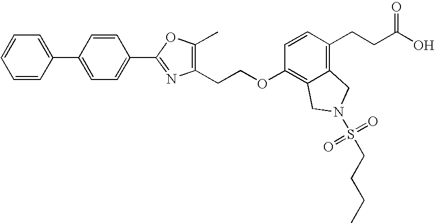

### Step A

### 4-Benzyloxy-7-(2-methoxycarbonylvinyl)-1,3-dihydroisoindole-2-carboxylic acid t-butyl ester

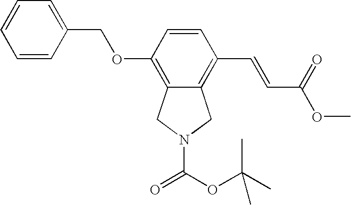

To a flame dried 100 mL round bottom flask under a nitrogen atmosphere are charged with 4-hydroxy-7-(2-methoxycarbonylvinyl)-1,3-dihydroisoindole-2-carboxylic acid t-butyl ester (2.0 g, 6.26 mmol), benzyl bromide (0.96 mL, 8.1 μmmol), and anhydrous DMF (20 mL). Cesium carbonate (3.06 g, 9.4 mmol) is added, and the reaction is stirred at ambient temperature for 18 h and then concentrated. The residue is taken up in 100 mL EtOAc, washed twice with brine, dried, and concentrated. The crude product is purified by medium pressure silica gel chromatography (15:85 EtOAc:hex) to give a yellow solid (1.9 g, 74%). MS (ES) m/e 410 (M+1).

### Step B

### 3-(7-Benzyloxy-2,3-dihydro-1H-isoindol-4-yl)acrylic acid methyl ester

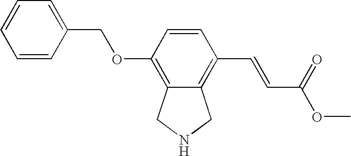

To 100 mL round bottom flask under a nitrogen atmosphere is charged with 4-benzyloxy-7-(2-methoxycarbonylvinyl)-1,3-dihydroisoindole-2-carboxylic acid t-butyl ester (1.0 g, 2.44 mmol) and anhydrous MeCl₂ (21 mL). To the mixture, trifluoroacetic acid (10.5 mL) is added. The mixture is stirred at ambient temperature for 16 h and concentrated. The residue is taken up in 50 mL of EtOAc, washed with 30 mL of saturated NaHCO₃, dried, and concentrated to give a dark oil (0.47 g, 62%). MS (ES) m/e 310 (M+1).

### Step C

### 3-[7-Benzyloxy-2-(butane-1-sulfonyl)-2,3-dihydro-1H-isoindol-4-yl]acrylic acid methyl ester

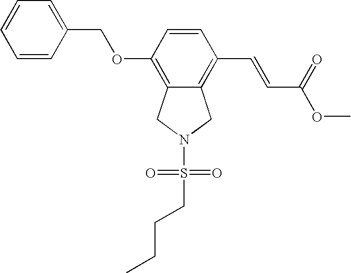

To a 50 mL round bottom flask under a nitrogen atmosphere are charged with 3-(7-benzyloxy-2,3-dihydro-1H-isoindol-4-yl)acrylic acid methyl ester (0.46 g, 1.49 mmol), triethylamine (0.62 mL, 4.47 mmol) and anhydrous MeCl₂ (10 mL). To the mixture, n-butylsulfonyl chloride (0.38 mL, 2.97 mmol) is added. The mixture is stirred at ambient temperature for 16 h. The mixture is diluted with 30 mL of MeCl₂, washed with brine, dried, and concentrated. The residue is purified using radial chromatography (2 mm silica gel plate, 5:95 to 35:65 EtOAc:hex) to give a yellow solid (0.41 g, 64%).

MS (ES) m/e 447 (M+NH₄).

### Step D

### 3-[2-(Butane-1-sulfonyl)-7-hydroxy-2,3-dihydro-1H-isoindol-4-yl]propionic acid methyl ester

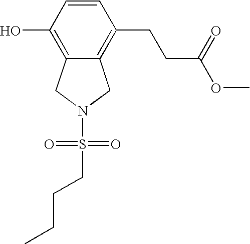

To a 500 mL Parr hydrogenation bottle is charged with 3-[7-benzyloxy-2-(butane-1-sulfonyl)-2,3-dihydro-1H-isoindol-4-yl]acrylic acid methyl ester (0.41 g, 0.95 mmol) and 20% palladium hydroxide on carbon (0.32 g) in 50 mL MeOH. The mixture is treated with hydrogen at 60 psi for 18 h, and then filtered through Celite and concentrated. The crude product is purified using radial chromatography (2 mm silica gel plate, 15:85 to 50:50 EtOAc:hex) to give a brown residue (0.090 g, 28%). MS (ES) m/e 342 (M+1).

### Step E

### 3-[7-[2-(2-Biphenyl-4-yl-5-methyloxazol-4-yl)ethoxy]-2-(butane-1-sulfonyl)-2,3-dihydro-1H-isoindol-4-yl]propionic acid methyl ester

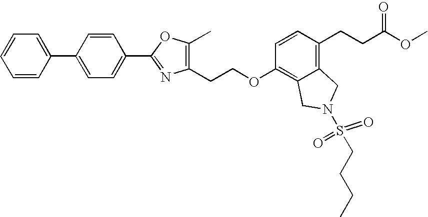

The title compound is prepared according to the procedure described in Example 140, Step H. MS (ES) m/e 603 (M+1).

### Step F

### 3-[7-[2-(2-Biphenyl-4-yl-5-methyloxazol-4-yl)ethoxy]-2-(butane-1-sulfonyl)-2,3-dihydro-1H-isoindol-4-yl]propionic acid

The title compound is prepared according to the procedure described in Example 140, Step I. MS (ES) m/e 589 (M+1).

### Example 174

### 3-[7-[2-(2-Biphenyl-3-yl-5-methyloxazol-4-yl)ethoxy]-2-(butane-1-sulfonyl)-2,3-dihydro-1H-isoindol-4-yl]propionic acid

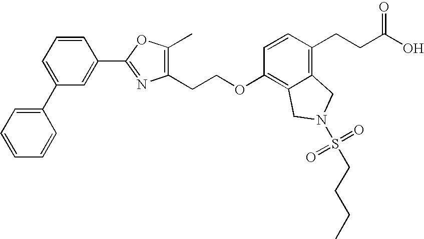

The title compound is prepared according to the procedure described in Example 173.

MS (ES) m/e 589 (M+1).

### Example 175

### 3-{2-(Butane-1-sulfonyl)-7-[2-(5-methyl-2-morpholin-4-ylthiazol-4-yl)ethoxy]-2,3-dihydro-1H-isoindol-4-yl}propionic acid

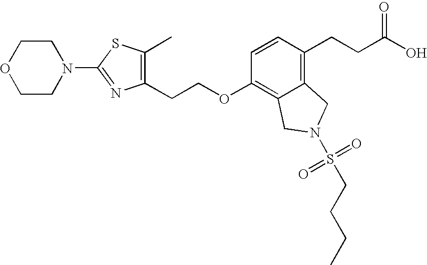

The title compound is prepared according to the procedure described in Example 173.

MS (ES) m/e 538 (M+1).

### Example 176

### 8-Methylcarboxy-ethyl-5-Hydroxy-3,4-dihydro-1H-isoquinoline-2-carboxylic acid tert-butyl ester

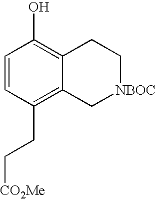

### Step A

### 5-Hydroxy-3,4-dihydro-1H-isoquinoline-2-carboxylic acid tert-butyl ester

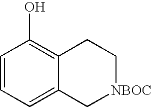

In 400 mL of acetic acid is mixed 50 g (516.7 mmol) of 5-hydroxyisoquinoline and 5.0 g of platinum oxide. The mixture is placed under 40 psig of H₂ and heated to 50° C. to dissolve 5-hydroxyisoquinoline. The mixture is then allowed cool to ambient temperature. After 23h, the mixture is filtered and HOAc is stripped to afford crude 5-hydroxy-3,4-dihydro-1H-isoquinoline (76.7 g) as tan solids containing HOAc (about 1 molar equivalent). The crude product is used without further purification. The crude 5-hydroxy isoquinoline (19.1 g) is dissolved in 150 mL of THF and 65 mL of H₂O. K₂CO₃ (21.2 g, 153.4 mmol) is added followed by di-t-butyldicarbonate (25.1 g, 115.0 mmol) in three portions. The mixture is heated to reflux. After 1 hour, TLC indicated complete reaction. The mixture is cooled to ambient temperature and partitioned between 50 mL of H₂O and 100 mL of EtOAc. The aqueous layer is extracted with 2×75 mL of EtOAc. The combined organic layer is washed with brine and dried over Na₂SO₄. Solvent is removed to afford orange-brown oil that is purified on 500g of silica gel by eluting with 2:1 hexanes:EtOAc. Product compound of 5-hydroxy-3,4-dihydro-1H-isoquinoline-2-carboxylic acid tert-butyl ester is isolated pure from two 500 mL fractions as 11.2 g white solids. Surrounding fractions are evaporated to give 11.8 g white solids containing excess BOC₂O. The material is recrystallized by dissolving in 35 mL of EtOAc at reflux. Hexane (70 mL) is added dropwise to the refluxing solution, and then the clear solution is cooled to rt. The white slurry is further cooled in an ice water bath for 90 minutes. Solids are filtered, washed with hexanes and dried under vacuum to afford 6.5 g of the title compound as white solids. ES/MS⁻ M−1 248.2.

### Step B

### 8-Bromo-5-benzyloxy-3,4-dihydro-1H-isoquinoline-2-carboxylic acid tert-butyl ester

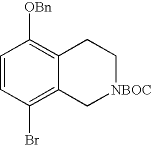

In a N₂ blanketed flask, 19.7 g (79.0 mmol) of 5-hydroxy-3,4-dihydro-1H-isoquinoline-2-carboxylic acid tert-butyl ester is dissolved in 300 mL of DMF, and 14.1 g (79.2 mmol) of N-bromosuccinimide is added in one portion. The solution is stirred overnight and quenched with 500 mL of H₂O. The resulting mixture is partitioned between 500 mL of EtOAc and 500 mL of H₂O. The aqueous layer is extracted twice with 400 mL of EtOAc, and the combined organic layer is washed with 300 mL of brine and dried over Na₂SO₄. Solvent is removed to afford 39.8 g crude product including residual DMF. The crude 8-bromo-5-hydroxy-3,4-dihydro-1H-isoquinoline-2-carboxylic acid tert-butyl ester is dissolved in 390 mL of acetone, and then 12.5 g (90.4 mmol) of K₂CO₃ is added followed by 10.3 mL (14.8 g, 86.6 mmol) of benzyl bromide. The mixture is stirred at reflux 20h, and then cooled to ambient temperature and filtered. The filtrate is evaporated, and the residue is partitioned between 50 mL of H₂O and 100 mL of EtOAc. The aqueous layer is extracted with EtOAc (2×100 mL). The combined organic layer is washed with brine and dried over Na₂SO₄. The solvent is removed to afford crude product as an orange-brown oil which is purified by silica gel chromatography to afford clean product of 8-bromo-5-benzyloxy-3,4-dihydro-1H-isoquinoline-2-carboxylic acid tert-butyl ester as 34.1 g of orange oil which crystallized upon standing. 1H NMR (250 MHz, CDCl₃) δ 7.3-7.15 (6H, m), 6.56 (1H, d, J=7.5 Hz), 4.94 (2H, s), 4.42 (2H, s), 3.53 (2H, t, J=5.7 Hz), 2.71 (2H, d, J=5.7 Hz), 1.42 (9H, s).

### Step C

### 5-Hydroxy-8-(2-methoxycarbonyl-ethyl)-3,4-dihydro-1H-isoquinoline-2-carboxylic acid tert-butyl ester

In a dry N₂ blanketed flask, 35.3 g (84.4 mmol) of 8-bromo-5-benzyloxy-3,4-dihydro-1H-isoquinoline-2-carboxylic acid tert-butyl ester is dissolved in 645 mL of propionitrile. Methyl acrylate (27.4 mL, 26.2 g, 304.3 mmol), 5.7 g (18.7 mmol) of tri-o-tolyl phosphine, and 29.4 mL (21.8 g, 168.8 mmol) of diopropylethylamine are added. The solution is degassed three times with vacuum/N₂. Palladium acetate (trimer) (2.1 g, 9.3 mmol) is added, and the reaction is heated at 90° C. overnight. The reaction is cooled to rt, and white solids are co-precipitated with black catalyst. After 2 h at rt and one hour in an ice-water bath, the solids are filtered, washed with 3:1 hexanes:EtOAc and dried. The gray solids are dissolved in 300 mL of methylene chloride, and the solution is filtered to remove the catalyst. The filtrate is evaporated to give 8-(2-methoxycarbonyl-ethyl)-5-benzyloxy-3,4-dihydro-1H-isoquinoline-2-carboxylic acid tert-butyl ester as 24.6 g of white solids (68.9%). About 23.9 g (56.43 mmol) of the above carboxylic acid tert-butyl ester is treated with 23.9 g of 5% Pd/C (Pd/C is slurred into the mixture with a minimum volume of EtOAc) in 1080 mL of MeOH under 50 psig of H₂. The reaction is stirred at rt for 5.5 h. The reaction is filtered, and solvent is removed by evaporation to afford 17.0 g of the title compound as a yellow oil that crystallizes on refrigeration (90%.) ES/MS⁻ M−1 334.2; 1H NMR (400 MHz, CDCl₃) δ 6.81 (1H, d, J=7.5 Hz), 6.59 (1H, d, J=7.5 Hz), 6.35 (1H, bs), 4.51 (3H, s) 3.7-3.5 (4H, m), 2.8-2.65 (4H, m), 2.49 (2H, t, J=5.6 Hz), 1.46 (9H, s).

What is claimed is:

1. A compound of formula I, 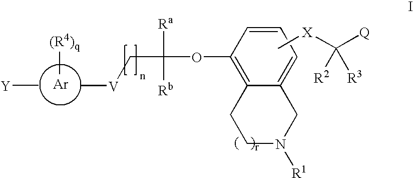
or a pharmaceutically acceptable salt or stereoisomer thereof, wherein:
V is: a bond or O;
X is: CH₂ or O;
Q is: C(O)OR⁵ or R^{5A};
n is: 0, 1, 2, 3 or 4;
m and q are each independently: 1, 2, 3 or 4;
p is: 1 or 2;
r is: 0 or 1; 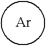
is: aryl, or 5- or 6-membered heteroaryl;
Y is: hydrogen,
aryloxy,
cycloalkyl,
heterocyclyl optionally being substituted with heteroaryl,
heteroaryl optionally being substituted with aryl,
(C₀-C₄)alkyl-aryl, wherein aryl being optionally substituted with aryl, aryloxy, heteroaryl, heterocyclyl or cycloalkyl, or
aryl-O(CH₂)ₘ-aryl;
wherein aryl, cycloalkyl, aryloxy, heteroaryl, and heterocyclyl are optionally substituted with one or more substituents independently selected from R⁶;
R^{a} and R^{b} are each independently: hydrogen or C₁-C₄ alkyl;
R¹ is: hydrogen,
alkyl,
aryl,
biphenyl,
C(O)ₚ-alkyl,
C(O)ₚ-alkynyl,
C(O)ₚ-alkoxy,
C(O)ₚ(C₀-C₅)alkyl-cycloalkyl,
C(O)ₚ-haloalkyl,
C(O)ₚ-biphenyl,
C(O)ₚ(C₀-C₅)alkyl-aryl,
C(O)ₚ(C₀-C₅)alkyl-heteroaryl,
C(O)ₚ(CH₂)ₘ-aryloxy,
C(O)ₚ(CH₂)ₘ—SR⁷,
C(O)ₚC(R⁷)(aryl)₂,
C(O)N(R⁷)₂,
S(O)ₚ-alkyl,
S(O)ₚ(C₀-C₆)alkyl-aryl or
S(O)ₚ(C₀-C₆)alkyl-heteroaryl;
wherein alkyl, aryl, aryloxy, alkynyl, alkoxy, cycloalkyl, heteroaryl and biphenyl being optionally substituted with one or more substituents independently selected from R^{6a};
R² and R³ are each independently: hydrogen, C₁-C₆ alkyl or C₁-C₆ alkoxy;
R⁴ is: hydrogen,
C₁-C₆ alkyl,
C₁-C₆ alkoxy,
halo,
haloalkyl or
haloalkyloxy;
R⁵ is: hydrogen, C₁-C₆ alkyl or aminoalkyl;
R^{5A} is: carboxamide, sulfonamide, acylsulfonamide, tetrazole, 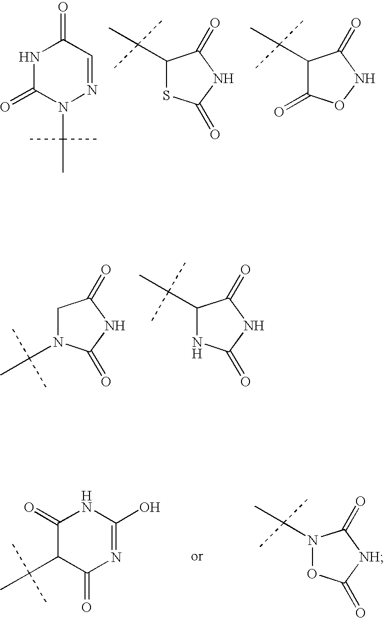
R⁶ and R^{6a} are each independently:
hydrogen,
halo,
nitro,
acyl,
cyano,
hydroxyl,
haloalkyl,
haloalkyloxy,
phenyl,
phenoxy,
benzyloxy,
thiophene,
pyridyl,
C₁-C₆ alkyl,
C₁-C₆ alkoxy,
S(O)₂R⁷,
S(O)₂N(R⁷)₂,
SR⁷ or
N(R⁷)₂; and
R⁷ is: hydrogen, C₁-C₆ alkyl or (C₀-C₆-alkyl)-aryl.

2. The compound of claim 1, wherein 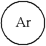
is phenyl, oxazolyl, thiazolyl, pyrazolyl or hydrofuranyl.

3. The compound of claim 1, wherein the compound is structural formula II, 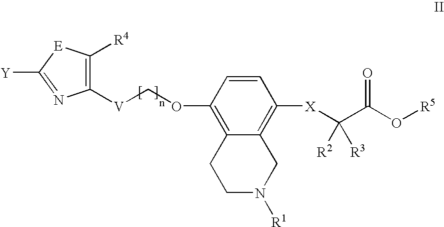
or a pharmaceutically acceptable salt or stereoisomer thereof, wherein:
E is O or S;
Y is:

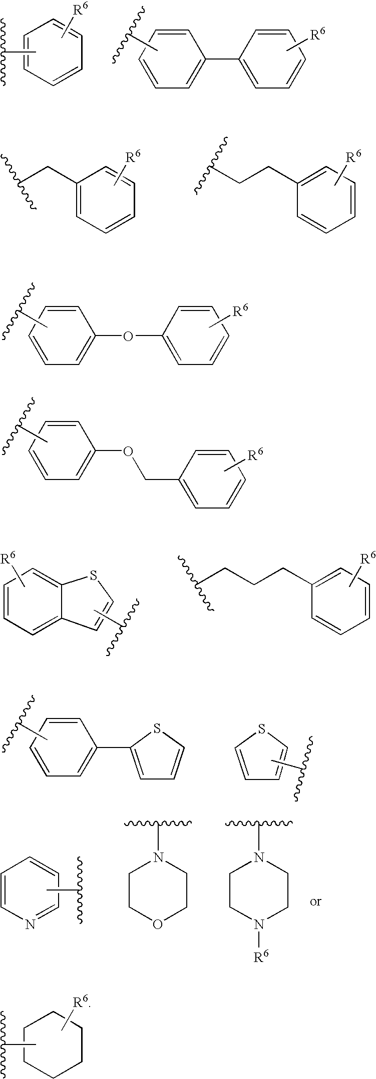

4. The compound of claim 3, wherein E is O.

5. The compound of claim 3, wherein the compound is structural formula III, 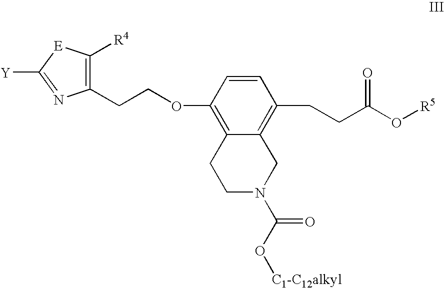
or a pharmaceutically acceptable salt or stereoisomer thereof, wherein:
R⁴ and R⁵ are each independently hydrogen, methyl or ethyl.

6. The compound of claim 5, wherein the compound is structural formula IV, 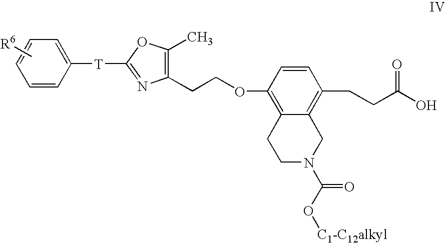
or a pharmaceutically acceptable salt or stereoisomer thereof, wherein:
T is: a bond, CH₂, (CH₂)₂, or (CH₂)₃,
R⁶ is: hydrogen, F, Br or CF₃, OCF₃, thiophene, benzyloxy, phenyl or pyridyl; and
C₁-C₁₂ alkyl is selected from the group consisting of:
methyl, ethyl, propyl, tert-butyl, butyl, isobutyloctane, hexyl, 2-hexylethyl, octyl, and 2,2-dimethylpropyl.

7. The compound of claim 3, wherein the compound is structural formula V, 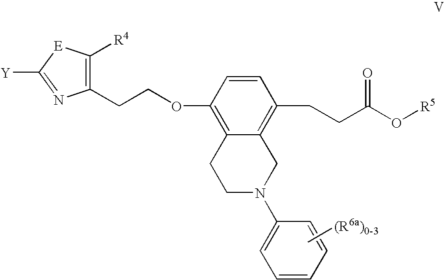
or a pharmaceutically acceptable salt or stereoisomer thereof, wherein:
R⁴ and R⁵ are each independently hydrogen, methyl or ethyl.

8. (canceled)

9. The compound of claim 3, wherein the compound is structural formula VII, 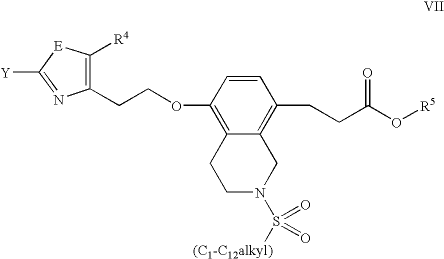
or a pharmaceutically acceptable salt or stereoisomer thereof, wherein:
R⁴ and R⁵ are each independently hydrogen, methyl or ethyl.

10. The compound of claim 9, wherein the compound is structural formula VIII, 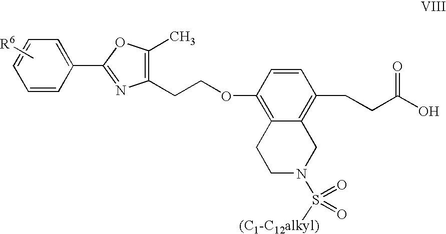
or a pharmaceutically acceptable salt or stereoisomer thereof, wherein:
R⁶ is: hydrogen, F, Br or CF₃, OCF₃, thiophene, benzyloxy, phenyl or pyridyl;
C₁-C₁₂ alkyl is selected from the group consisting of:
methyl, ethyl, propyl, tert-butyl, butyl, isobutyl, octane, and 2,2-dimethylpropyl.

11. The compound of claim 3, wherein the compound is structural formula IX, 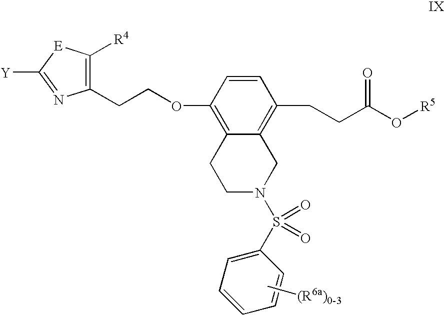
or a pharmaceutically acceptable salt or stereoisomer thereof, wherein:
R⁴ and R⁵ are each independently hydrogen or methyl.

12. (canceled)

13. The compound of claim 1, wherein the compound is formula XI, 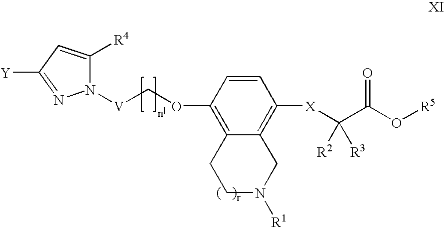
or a pharmaceutically acceptable salt or stereoisomer thereof, wherein:
Y is:

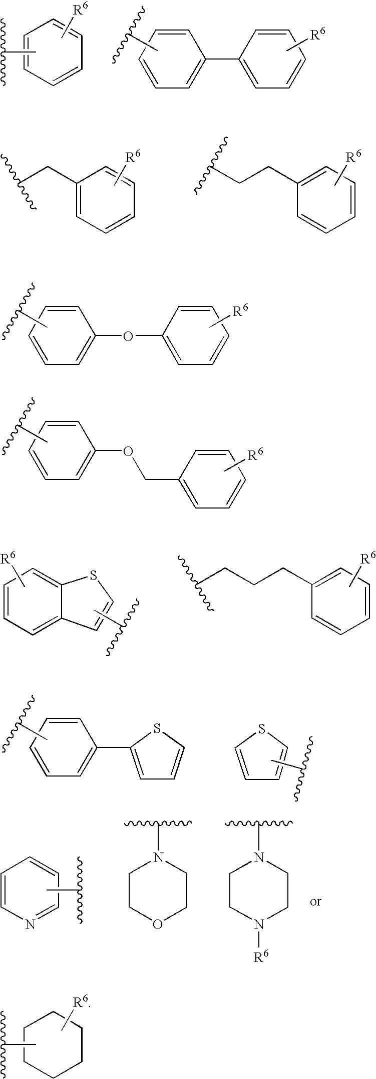

14. The compound of claim 1, wherein the compound is structural formula XII, 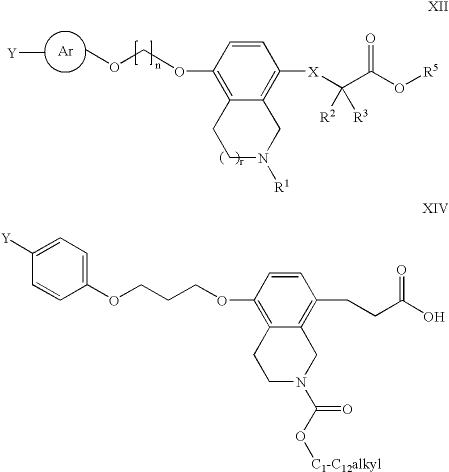
or a pharmaceutically acceptable salt or stereoisomer thereof, wherein:
Y is: phenyl or phenoxy; and
C₁-C₁₂ alkyl is selected from the group consisting of:
methyl, ethyl, propyl, tert-butyl, butyl, isobutyl, octyl and 2,2-dimethylpropyl. 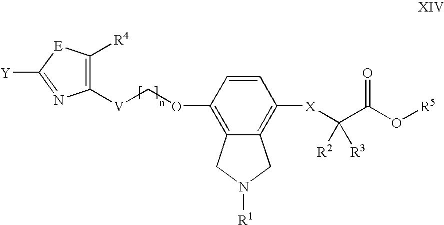
or a pharmaceutically acceptable salt or stereoisomer thereof, wherein:
E is O or S. 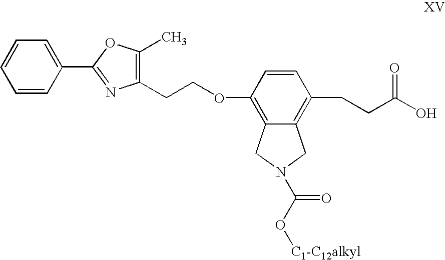
or a pharmaceutically acceptable salt or stereoisomer thereof, wherein:
C₁-C₁₂ alkyl is selected from the group consisting of:
methyl, ethyl, propyl, tert-butyl, butyl, isobutyl, octyl, hexyl, 2-hexylethyl, and 2,2-dimethylpropyl. 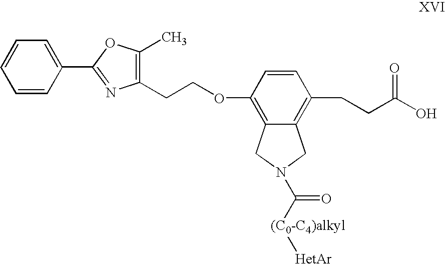
or a pharmaceutically acceptable salt or stereoisomer thereof, wherein: 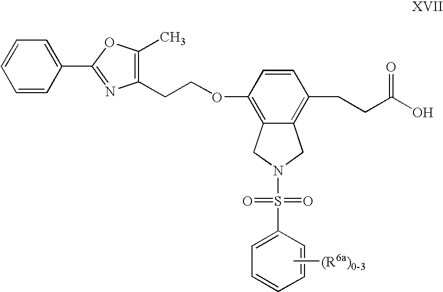
or a pharmaceutically acceptable salt or stereoisomer thereof, wherein:
R^{6a} is each independently selected from the group consisting of:
methyl, ethyl, propyl, isopropyl, tert-butyl, pentyl, 1,1-dimethylpropyl, methoxy, butoxy, acetyl, propionyl, phenyl, methanesulfonyl, F, Cl, Br, CF₃, OCF₃, nitro, cyano, dimethylamino and ethylsunfanyl. 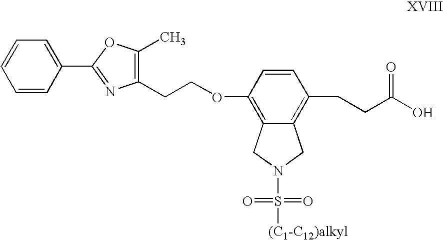
or a pharmaceutically acceptable salt or stereoisomer thereof, wherein:
C₁-C₁₂ alkyl is selected from the group consisting of:
methyl, ethyl, propyl, tert-butyl, butyl, isobutyl, octyl, hexyl, 2-hexylethyl, and 2,2-dimethylpropyl.

15. (canceled)

16. (canceled)

17. (canceled)

18. (canceled)

19. (canceled)

20. (canceled)

21. A pharmaceutical composition comprising a pharmaceutically acceptable carrier and a compound of claim 1 or a pharmaceutically acceptable salt thereof.

22. (canceled)

23. (canceled)

24. (canceled)

25. (canceled)

26. (canceled)

27. A method for lowering blood-glucose comprising the step of administering an effective amount of a compound of claim 1.

28. (canceled)

29. (canceled)

30. (canceled)

31. (canceled)

32. (canceled)

33. (canceled)
